SEQUENCE LISTING (52 sequences)

SEQ ID NO: 1 - DNA, 30 bp, Artificial sequence
  Description of the artificial sequence primer
ccggatccatgagtgcaatagaaaatttcg

SEQ ID NO: 2 - DNA, 27 bp, Artificial sequence
  Description of the artificial sequence primer
ccaagcttacaccaggctcttcaagcg

SEQ ID NO: 3 - DNA, 2576 bp, Escherichia coli
ccggatccatgagtgcaatagaaaatttcgacgcccatacgcccatgatgcagcagtatctcaggctgaaagcccagcatcccgagatcctgctgttttaccggatgggtgatttttatgaactgttttatgacgacgcaaaacgcgcgtcgcaactgctggatatttcactgaccaaacgcggtgcttcggcgggagagccgatcccgatggcggggattccctaccatgcggtggaaaactatctcgccaaactggtgaatcagggagagtccgttgccatctgcgaacaaattggcgatccggcgaccagcaaaggtccggttgagcgcaaagttgtgcgtatcgttacgccaggcaccatcagcgatgaagccctgttgcaggagcgtcaggacaacctgctggcggctatctggcaggacagcaaaggtttcggctacgcgacgctggatatcagttccgggcgttttcgcctgagcgaaccggctgaccgcgaaacgatggcggcagaactgcaacgcactaatcctgcggaactgctgtatgcagaagattttgctgaaatgtcgttaattgaaggccgtcgcggcctgcgccgtcgcccgctgtgggagtttgaaatcgacaccgcgcgccagcagttgaatctgcaatttgggacccgcgatctggtcggttttggcgtcgagaacgcgccgcgcggactttgtgctgccggttgtctgttgcagtatgcgaaagatacccaacgtacgactctgccgcatattcgttccatcaccatggaacgtgagcaggacagcatcattatggatgccgcgacgcgtcgtaatctggaaatcacccagaacctggcgggtggtgcggaaaatacgctggcttctgtgctcgactgcaccgtcacgccgatgggcagccgtatgctgaaacgctggctgcatatgccagtgcgcgatacccgcgtgttgcttgagcgccagcaaactattggcgcattgcaggatttcaccgccgggctacagccggtactgcgtcaggtcggcgacctggaacgtattctggcacgtctggctttacgaactgctcgcccacgcgatctggcccgtatgcgccacgctttccagcaactgccggagctgcgtgcgcagttagaaactgtcgatagtgcaccggtacaggcgctacgtgagaagatgggcgagtttgccgagctgcgcgatctgctggagcgagcaatcatcgacacaccgccggtgctggtacgcgacggtggtgttatcgcatcgggctataacgaagagctggatgagtggcgcgcgctggctgacggcgcgaccgattatctggagcgtctggaagtccgcgagcgtgaacgtaccggcctggacacgctgaaagttggctttaatgcggtgcacggctactacattcaaatcagccgtgggcaaagccatctggcacccatcaactacatgcgtcgccagacgctgaaaaacgccgagcgctacatcattccagagctaaaagagtacgaagataaagttctcacctcaaaaggcaaagcactggcactggaaaaacagctttatgaagagctgttcgacctgctgttgccgcatctggaagcgttgcaacagagcgcgagcgcgctggcggaactcgacgtgctggttaacctggcggaacgggcctataccctgaactacacctgcccgaccttcattgataaaccgggcattcgcattaccgaaggtcgccatccggtagttgaacaagtactgaatgagccatttatcgccaacccgctgaatctgtcgccgcagcgccgcatgttgatcatcaccggtccgaacatgggcggtaaaagtacctatatgcgccagaccgcactgattgcgctgatggcctacatcggcagctatgtaccggcacaaaaagtcgagattggacctatcgatcgcatctttacccgcgtaggcgcggcagatgacctggcgtccgggcgctcaacctttatggtggagatgactgaaaccgccaatattttacataacgccaccgaatacagtctggtgttaatggatgagatcgggcgtggaacgtccacctacgatggtctgtcgctggcgtgggcgtgcgcggaaaatctggcgaataagattaaggcattgacgttatttgctacccactatttcgagctgacccagttaccggagaaaatggaaggcgtcgctaacgtgcatctcgatgcactggagcacggcgacaccattgcctttatgcacagcgtgcaggatggcgcggcgagcaaaagctacggcctggcggttgcagctctggcaggcgtgccaaaagaggttattaagcgcgcacggcaaaagctgcgtgagctggaaagcatttcgccgaacgccgccgctacgcaagtggatggtacgcaaatgtctttgctgtcagtaccagaagaaacttcgcctgcggtcgaagctctggaaaatcttgatccggattcactcaccccgcgtcaggcgctggagtggatttatcgcttgaagagcctggtgtaagcttgg

SEQ ID NO: 4 - DNA, 3462 bp, Artificial sequence
  Description of the artificial sequence plasmid pQE30
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccgcatgcgagctcggtaccccgggtcgacctgcagccaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 5 - DNA, 5986 bp, Artificial sequence
  Description of the artificial sequence plasmid pQE30-mutS
ctcgagaaatcataaaaaatttatttgctttgtgagcggataacaattataatagattcaattgtgagcggataacaatttcacacagaattcattaaagaggagaaattaactatgagaggatcgcatcaccatcaccatcacggatccatgagtgcaatagaaaatttcgacgcccatacgcccatgatgcagcagtatctcaggctgaaagcccagcatcccgagatcctgctgttttaccggatgggtgatttttatgaactgttttatgacgacgcaaaacgcgcgtcgcaactgctggatatttcactgaccaaacgcggtgcttcggcgggagagccgatcccgatggcggggattccctaccatgcggtggaaaactatctcgccaaactggtgaatcagggagagtccgttgccatctgcgaacaaattggcgatccggcgaccagcaaaggtccggttgagcgcaaagttgtgcgtatcgttacgccaggcaccatcagcgatgaagccctgttgcaggagcgtcaggacaacctgctggcggctatctggcaggacagcaaaggtttcggctacgcgacgctggatatcagttccgggcgttttcgcctgagcgaaccggctgaccgcgaaacgatggcggcagaactgcaacgcactaatcctgcggaactgctgtatgcagaagattttgctgaaatgtcgttaattgaaggccgtcgcggcctgcgccgtcgcccgctgtgggagtttgaaatcgacaccgcgcgccagcagttgaatctgcaatttgggacccgcgatctggtcggttttggcgtcgagaacgcgccgcgcggactttgtgctgccggttgtctgttgcagtatgcgaaagatacccaacgtacgactctgccgcatattcgttccatcaccatggaacgtgagcaggacagcatcattatggatgccgcgacgcgtcgtaatctggaaatcacccagaacctggcgggtggtgcggaaaatacgctggcttctgtgctcgactgcaccgtcacgccgatgggcagccgtatgctgaaacgctggctgcatatgccagtgcgcgatacccgcgtgttgcttgagcgccagcaaactattggcgcattgcaggatttcaccgccgggctacagccggtactgcgtcaggtcggcgacctggaacgtattctggcacgtctggctttacgaactgctcgcccacgcgatctggcccgtatgcgccacgctttccagcaactgccggagctgcgtgcgcagttagaaactgtcgatagtgcaccggtacaggcgctacgtgagaagatgggcgagtttgccgagctgcgcgatctgctggagcgagcaatcatcgacacaccgccggtgctggtacgcgacggtggtgttatcgcatcgggctataacgaagagctggatgagtggcgcgcgctggctgacggcgcgaccgattatctggagcgtctggaagtccgcgagcgtgaacgtaccggcctggacacgctgaaagttggctttaatgcggtgcacggctactacattcaaatcagccgtgggcaaagccatctggcacccatcaactacatgcgtcgccagacgctgaaaaacgccgagcgctacatcattccagagctaaaagagtacgaagataaagttctcacctcaaaaggcaaagcactggcactggaaaaacagctttatgaagagctgttcgacctgctgttgccgcatctggaagcgttgcaacagagcgcgagcgcgctggcggaactcgacgtgctggttaacctggcggaacgggcctataccctgaactacacctgcccgaccttcattgataaaccgggcattcgcattaccgaaggtcgccatccggtagttgaacaagtactgaatgagccatttatcgccaacccgctgaatctgtcgccgcagcgccgcatgttgatcatcaccggtccgaacatgggcggtaaaagtacctatatgcgccagaccgcactgattgcgctgatggcctacatcggcagctatgtaccggcacaaaaagtcgagattggacctatcgatcgcatctttacccgcgtaggcgcggcagatgacctggcgtccgggcgctcaacctttatggtggagatgactgaaaccgccaatattttacataacgccaccgaatacagtctggtgttaatggatgagatcgggcgtggaacgtccacctacgatggtctgtcgctggcgtgggcgtgcgcggaaaatctggcgaataagattaaggcattgacgttatttgctacccactatttcgagctgacccagttaccggagaaaatggaaggcgtcgctaacgtgcatctcgatgcactggagcacggcgacaccattgcctttatgcacagcgtgcaggatggcgcggcgagcaaaagctacggcctggcggttgcagctctggcaggcgtgccaaaagaggttattaagcgcgcacggcaaaagctgcgtgagctggaaagcatttcgccgaacgccgccgctacgcaagtggatggtacgcaaatgtctttgctgtcagtaccagaagaaacttcgcctgcggtcgaagctctggaaaatcttgatccggattcactcaccccgcgtcaggcgctggagtggatttatcgcttgaagagcctggtgtaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 6 - DNA, 36 bp, Artificial sequence
  Description of the artificial sequence “AT” oligonucleotide
tggctagagatgatccgcactttaacttccgtatgc

SEQ ID NO: 7 - DNA, 36 bp, Artificial sequence
  Description of the artificial sequence “GT” oligonucleotide
tggctagagatgatccgcgctttaacttccgtatgc

SEQ ID NO: 8 - DNA, 6648 bp, Artificial sequence
  Description of the artificial sequence plasmid pMAL-c2x
ccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcacttcaccaacaaggaccatagcatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatcctctagagtcgacctgcaggcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcagcttggctgttttggcggatgagataagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 9 - DNA, 9191 bp, Artificial sequence
  Description of the artificial sequence pMALc2x-mutS plasmid
ccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcacttcaccaacaaggaccatagcatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccactatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatttcagaattcggatccggaatgagtgcaatagaaaatttcgacgcccatacgcccatgatgcagcagtatctcaggctgaaagcccagcatcccgagatcctgctgttttaccggatgggtgatttttatgaactgttttatgacgacgcaaaacgcgcgtcgcaactgctggatatttcactgaccaaacgcggtgcttcggcgggagagccgatcccgatggcggggattccctaccatgcggtggaaaactatctcgccaaactggtgaatcagggagagtccgttgccatctgcgaacaaattggcgatccggcgaccagcaaaggtccggttgagcgcaaagttgtgcgtatcgttacgccaggcaccatcagcgatgaagccctgttgcaggagcgtcaggacaacctgctggcggctatctggcaggacagcaaaggtttcggctacgcgacgctggatatcagttccgggcgttttcgcctgagcgaaccggctgaccgcgaaacgatggcggcagaactgcaacgcactaatcctgcggaactgctgtatgcagaagattttgctgaaatgtcgttaattgaaggccgtcgcggcctgcgccgtcgcccgctgtgggagtttgaaatcgacaccgcgcgccagcagttgaatctgcaatttgggacccgcgatctggtcggttttggcgtcgagaacgcgccgcgcggactttgtgctgccggttgtctgttgcagtatgcgaaagatacccaacgtacgactctgccgcatattcgttccatcaccatggaacgtgagcaggacagcatcattatggatgccgcgacgcgtcgtaatctggaaatcacccagaacctggcgggtggtgcggaaaatacgctggcttctgtgctcgactgcaccgtcacgccgatgggcagccgtatgctgaaacgctggctgcatatgccagtgcgcgatacccgcgtgttgcttgagcgccagcaaactattggcgcattgcaggatttcaccgccgggctacagccggtactgcgtcaggtcggcgacctggaacgtattctggcacgtctggctttacgaactgctcgcccacgcgatctggcccgtatgcgccacgctttccagcaactgccggagctgcgtgcgcagttagaaactgtcgatagtgcaccggtacaggcgctacgtgagaagatgggcgagtttgccgagctgcgcgatctgctggagcgagcaatcatcgacacaccgccggtgctggtacgcgacggtggtgttatcgcatcgggctataacgaagagctggatgagtggcgcgcgctggctgacggcgcgaccgattatctggagcgtctggaagtccgcgagcgtgaacgtaccggcctggacacgctgaaagttggctttaatgcggtgcacggctactacattcaaatcagccgtgggcaaagccatctggcacccatcaactacatgcgtcgccagacgctgaaaaacgccgagcgctacatcattccagagctaaaagagtacgaagataaagttctcacctcaaaaggcaaagcactggcactggaaaaacagctttatgaagagctgttcgacctgctgttgccgcatctggaagcgttgcaacagagcgcgagcgcgctggcggaactcgacgtgctggttaacctggcggaacgggcctataccctgaactacacctgcccgaccttcattgataaaccgggcattcgcattaccgaaggtcgccatccggtagttgaacaagtactgaatgagccatttatcgccaacccgctgaatctgtcgccgcagcgccgcatgttgatcatcaccggtccgaacatgggcggtaaaagtacctatatgcgccagaccgcactgattgcgctgatggcctacatcggcagctatgtaccggcacaaaaagtcgagattggacctatcgatcgcatctttacccgcgtaggcgcggcagatgacctggcgtccgggcgctcaacctttatggtggagatgactgaaaccgccaatattttacataacgccaccgaatacagtctggtgttaatggatgagatcgggcgtggaacgtccacctacgatggtctgtcgctggcgtgggcgtgcgcggaaaatctggcgaataagattaaggcattgacgttatttgctacccactatttcgagctgacccagttaccggagaaaatggaaggcgtcgctaacgtgcatctcgatgcactggagcacggcgacaccattgcctttatgcacagcgtgcaggatggcgcggcgagcaaaagctacggcctggcggttgcagctctggcaggcgtgccaaaagaggttattaagcgcgcacggcaaaagctgcgtgagctggaaagcatttcgccgaacgccgccgctacgcaagtggatggtacgcaaatgtctttgctgtcagtaccagaagaaacttcgcctgcggtcgaagctctggaaaatcttgatccggattcactcaccccgcgtcaggcgctggagtggatttatcgcttgaagagcctggtgtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcagcttggctgttttggcggatgagataagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttctccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaccccggttgataatcagaaaagccccaaaaacaggaagattgtataagcaaatatttaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 10 - DNA, 38 bp, Artificial sequence
  Description of the artificial sequence “sense” oligonucleotide
aagcatacggaagttaaagtgcggatcatctctagcca

SEQ ID NO: 11 - DNA, 36 bp, Artificial sequence
  Description of the artificial sequence “sense” oligonucleotide
aagcatacggaagttaaagtgcggatcatctctagc

SEQ ID NO: 12 - DNA, 36 bp, Artificial sequence; identical to SEQ ID NO: 6
  Description of the artificial sequence “AT” oligonucleotide

SEQ ID NO: 13 - DNA, 36 bp, Artificial sequence; identical to SEQ ID NO: 7
  Description of the artificial sequence “GT” oligonucleotide

SEQ ID NO: 14 - DNA, 36 bp, Artificial sequence
  Description of the artificial sequence “AA” oligonucleotide
tggctagagatgatccgcacattaacttccgtatgc

SEQ ID NO: 15 - DNA, 36 bp, Artificial sequence
  Description of the artificial sequence “AG” oligonucleotide
tggctagagatgatccgcaatttaacttccgtatgc

SEQ ID NO: 16 - DNA, 36 bp, Artificial sequence
  Description of the artificial sequence “CA” oligonucleotide
tggctagagatgatccgcaccttaacttccgtatgc

SEQ ID NO: 17 - DNA, 36 bp, Artificial sequence
  Description of the artificial sequence “CC” oligonucleotide
tggctagagatgatccccactttaacttccgtatgc

SEQ ID NO: 18 - DNA, 36 bp, Artificial sequence
  Description of the artificial sequence “CT” oligonucleotide
tggctagagatgatccgccctttaacttccgtatgc

SEQ ID NO: 19 - DNA, 36 bp, Artificial sequence
  Description of the artificial sequence “GG” oligonucleotide
tggctagagatgatccgcagtttaacttccgtatgc

SEQ ID NO: 20 - DNA, 36 bp, Artificial sequence
  Description of the artificial sequence “TT” oligonucleotide
tggctagagatgatccgctctttaacttccgtatgc

SEQ ID NO: 21 - DNA, 37 bp, Artificial sequence
  Description of the artificial sequence “ins+1T” oligonucleotide
tggctagagatgatccgcacttttaacttccgtatgc

SEQ ID NO: 22 - DNA, 38 bp, Artificial sequence
  Description of the artificial sequence “ins+2T” oligonucleotide
tggctagagatgatccgcactttttaacttccgtatgc

SEQ ID NO: 23 - DNA, 39 bp, Artificial sequence
  Description of the artificial sequence “ins+3T” oligonucleotide
tggctagagatgatccgcacttttttaacttccgtatgc

SEQ ID NO: 24 - DNA, 39 bp, Homo sapiens
aagatcttcagctgacctagttccaatcttttcttttat

SEQ ID NO: 25 - DNA, 39 bp, Homo sapiens
aaataaaagaaaagattggaactaggtcagctgaagatc

SEQ ID NO: 26 - DNA, 39 bp, Homo sapiens
aaataaaagaaaagattggagctaggtcagctgaagatc

SEQ ID NO: 27 - DNA, 39 bp, Homo sapiens
aaggtcgcgggatgcggctggatggggcgtgtgcccggg

SEQ ID NO: 28 - DNA, 39 bp, Homo sapiens
agcccgggcacacgccccatccagccgcatcccgcgacc

SEQ ID NO: 29 - DNA, 39 bp, Homo sapiens
agcccgggcacacgccccattcagccgcatcccgcgacc

SEQ ID NO: 30 - DNA, 39 bp, Homo sapiens
aaatgttattacggctaattgtgctcactgtacttggaa

SEQ ID NO: 31 - DNA, 39 bp, Homo sapiens
cattccaagtacagtgagcacaattagccgtaataacat

SEQ ID NO: 32 - DNA, 39 bp, Homo sapiens
cattccaagtacagtgagcataattagccgtaataacat

SEQ ID NO: 33 - DNA, 39 bp, Homo sapiens
aactatagtattctttatcatacatgtctctggcaagac

SEQ ID NO: 34 - DNA, 39 bp, Homo sapiens
tggtcttgccagagacatgtatgataaagaatactatag

SEQ ID NO: 35 - DNA, 39 bp, Homo sapiens
tggtcttgccagagacatgtgtgataaagaatactatag

SEQ ID NO: 36 - DNA, 39 bp, Homo sapiens
aacctttctccaaaatggctggtcgtacatatggaacag

SEQ ID NO: 37 - DNA, 39 bp, Homo sapiens
acctgttccatatgtacgaccagccattttggagaaagg

SEQ ID NO: 38 - DNA, 39 bp, Homo sapiens
acctgttccatatgtacgactagccattttggagaaagg

SEQ ID NO: 39 - DNA, 38 bp, Homo sapiens
aaagttcctgcatgggcggcatgaaccggaggcccatc

SEQ ID NO: 40 - DNA, 38 bp, Homo sapiens
aggatgggcctccggttcatgccgcccatgcaggaact

SEQ ID NO: 41 - DNA, 38 bp, Homo sapiens
aggatgggcctccggttcatgctgcccatgcaggaact

SEQ ID NO: 42 - DNA, 39 bp, Homo sapiens
aaataagatcaaataaaggtgaatctgagagccatgcaa

SEQ ID NO: 43 - DNA, 39 bp, Homo sapiens
ccttgcatggctctcagattcacctttatttgatcttat

SEQ ID NO: 44 - DNA, 39 bp, Homo sapiens
ccttgcatggctctcagatttacctttatttgatcttat

SEQ ID NO: 45 - DNA, 19 bp, Artificial sequence
  Description of the artificial sequence primer
ccttactgcctcttgcttc

SEQ ID NO: 46 - DNA, 20 bp, Artificial sequence
  Description of the artificial sequence primer
tgaatctgaggcataactgc

SEQ ID NO: 47 - DNA, 73 bp, Homo sapiens
agtggtaatctactgggacggaacagctttgaggtgcgtgtttgtgcctgtcctgggagagaccggcgcacag

SEQ ID NO: 48 - DNA, 19 bp, Artificial sequence
  Description of the artificial sequence primer
ccttactgcctcttgcttc

SEQ ID NO: 49 - DNA, 20 bp, Artificial sequence
  Description of the artificial sequence primer
tgaatctgaggcataactgc

SEQ ID NO: 50 - DNA, 49 bp, Artificial sequence
  Description of the artificial sequence hairpin Oligonucleotide
attcgatcggggcggggcgagctttttgctcgccttgccccgatcgaat

SEQ ID NO: 51 - DNA, 48 bp, Artificial sequence
  Description of the artificial sequence hairpin oligonucleotide
attcgatcggggcggggcgagcttttgctcgccccgccccgatcgaat

SEQ ID NO: 52 - DNA, 33 bp, Artificial sequence
  Description of the artificial sequence primer
ccggatccggaatgagtgcaatagaaaatttcg

(19) United States
(12) Patent Application Publication
(10) Pub. No.: US 2004/0110161 A1
(43) Pub. Date: Jun. 10, 2004

(54) Method for detecting mutations in nucleotide sequences

(76) Inventors: Andreas Kappel, Konigstein (DE); Thomas Polakowski, Berlin (DE); Marc Pignot, Ebersbeg (DE); Norbert Windhab, Hofheim (DE); Heike Behrensdorf, Frankfurt (DE); Jochen Muth, Frankfurt (DE)

Correspondence Address: O'MELVENY & MEYERS, 114 PACIFICA, SUITE 100, IRVINE, CA, 92618, US

(21) Appl. No.: 10/343,859

(22) PCT Filed: Jul. 13, 2001
(86) PCT No.: PCT/EP01/08127

(30) Foreign Application Priority Data
Aug. 4, 2000 (DE) 100 38 237.1

(51) Int. Cl. C12Q 1/68

(52) U.S. Cl. 435/6

(57) ABSTRACT

The invention relates to a method for simultaneously detecting mutations in different nucleotide sequences and for determining the transcription rate of mutated and non-mutated nucleotide sequences. The inventive method comprises the following steps: hybridizing single-stranded sample nucleotide sequences to single-stranded reference nucleotide sequences, fixating, before or during hybridization, single-stranded reference nucleotide sequences or single-stranded sample nucleotide sequences, or fixating, after or during hybridization, heteroduplices from reference and sample nucleotide sequences on an electronically addressable surface, incubating them with a substrate that recognizes heteroduplex mismatches, and detecting the substrate bonds.

The present invention relates to a method which can be used for detecting mutations in parallel in different nucleotide sequences, with the method additionally making it possible to determine the transcription rate of mutated and nonmutated nucleotide sequences.

It is known that the DNA sequences of most of the genes in the human body are transcribed into protein sequences. In this connection, the activity of a protein, for example an enzyme, in different individuals or cell types is determined by several factors. Firstly, the transcription activity of the given gene determines how many copies of the protein are present in a cell. Secondly, mutations can affect the activity of a protein. Thus, a decrease in transcription rate or a repressing mutation can lead to protein activity being reduced (“loss-of-function”) whereas an increase in transcription rate or one of the activating mutations, which occur rarely, lead to protein activity being increased (“gain-of-function”). Other factors, such as translation regulation and post-translational modifications, can likewise influence the activity of proteins over and above this. Although two individuals or cell types are almost identical at the genomic level, these factors ultimately determine large differences with regard to anatomy, physiology, pathology and the reaction to pharmacological active compounds. Since most diseases are caused by a change in protein activity, and since pharmaceutical active compounds regulate the activity of particular proteins, an investigation of the utranscription“and umutation” factors is very particularly suitable for clinical diagnosis and also for identifying pharmacological targets. Thus, differences in the level at which particular genes are expressed can determine different reactions to drugs in different patients. However, only a few genes, such as the MDR gene (multi drug-resistance gene) (S. Akayama et al., Hum. Cell 12, 95-102, 1999), have so far been investigated in detail for this purpose. By contrast, a number of drugs are known where mutations in particular genes lead to a drug not being tolerated or to the therapy failing (W. H. Anderson, New Horizons 7, 262-269, 1999). Prior to therapy with these active compounds, patients should, therefore, be examined for the presence of the given mutation in order to prevent incorrect medication. However, this is nowadays only possible in isolated cases since success is seldom achieved in assigning incompatibility to a drug to a specific genotype. This is due, in particular, to the lack of suitable test methods which can be used to carry out genotype analyses in a high-throughput process and in reasonable time. However, it would be very desirable to develop such test methods since in the USA alone, for example, approx. 100,000 people die every year as a result of drug intolerance. In addition to this, the consistent use of such test methods would make it possible to approve drugs whose failure in a patient group can be assigned to a particular mutation. For this, such a test method must identify this patient group reliably in order to be able to rule out any administration of this drug to the group. For this, candidate genes from many patients would have to be examined prospectively for the presence of any mutation correlating with intolerance to a drug or with the drug being inactive. Thus, the determination of mutations and transcription rates could represent an important tool when deciding for or against a therapy with a given active compound. The consideration or investigation of the genotypic peculiarities of individuals in connection with therapy or medical check-ups is termed Upharmacogenomicso and is likely to constitute a crucial part of future medical activities. In this connection, it will be of crucial importance to be able to establish test methods which, on the one hand, ensure high sample throughput in reasonable time and, on the other hand, supply extremely reliable test results.

So far, changes in the transcription rate of particular genes have been determined using different methods for detecting RNA, such as Northern blotting, RNAse protection, RT-PCR and high density filter arrays, or indirectly using methods, such as Western blotting, RIA or ELISA, for detecting the proteins which are formed. While all these methods have proved to be suitable for examining single samples, they do not permit any high sample throughput.

A new method, based on DNA chip technology, for the highly parallel analysis of the expression profiles of multiple genes has been developed for the purpose of raising sample throughput (D. J. Lockhart and E. A. Winzeler, Nature 405, 827ff, 2000). In this method, DNA sequences with which the mRNA or cDNA from a biological sample can hybridize in a sequence-specific manner, and then be readily detected, are applied to the chip surface.

The company Nanogen (San Diego/USA) have already developed, and published, several methods for achieving an accelerated hybridization of nucleotide sequences, and consequently a decrease in measurement times, with these methods enabling the user to prepare user-defined DNA chips by addressing DNA sequences electronically (R. G. Sosnowski et al., Proc. Natl. Acad. Sci. USA, 94, 1119-1123, 1997) (U.S. Pat. No. 6,068,818; U.S. Pat. No. 6,051,380; U.S. Pat. No. 6,017,696; U.S. Pat. No. 5,965,452; U.S. Pat. No. 5,849,486; U.S. Pat. No. 5,632,957; U.S. Pat. No. 5,605,662). In these methods, the DNA, which is usually conjugated with biotin, is moved through an electric field onto a test electrode and, at the electrode, binds with high affinity to streptavidin which is present in the permeation layer which is located on top of the electrode. The subsequent hybridization is likewise made possible by electronic addressing within the shortest possible time. Each single one of the test electrodes, which are usually 99 in number, in such a chip can be actuated individually; in contrast to other chip technologies, this makes it possible to process several samples independently of each other. While the described methods are suitable for protecting nucleotide sequences in a sample, it is not possible to detect a mutation at high sample throughput using the methods described in the above publications on their own.

When developing new test methods for identifying mutations, primary consideration is given to detecting point mutations. Point mutations (single nucleotide polymorphisms, SNPs) constitute the most frequent cause of genetic variation within the human population and occur at a frequency of from 0.5 to 10 per 1000 basepairs (A. J. Schafer and J. R. Hawkins, Nature Biotechnol, 16, 33-39, 1998). However, it remains difficult to correlate SNPs with phenomenological effects. Thus, despite many SNPs having been found, it has so far only been possible to assign a few of them to particular drug intolerance reactions (W. H. Anderson, New Horizons 7, 262-269, 1999). A known example is a mutation in the factor IX propeptide, which mutation leads to heavy bleeding in connection with anticoagulant therapy with coumarin (J. Oldenburg et al., Brit. J. Hematol. 98 (1997), 240-244). However, the sequence data which were obtained during the course of the human genome project nowadays in principle make it possible to rapidly assign an identified SNP to a particular drug intolerance. For this reason, different methods have been developed for detecting previously unknown SNPs (D. J. Fu et al., Nature Biotechnol. 1998, 16, 381-384; Fan et al., Mut. Res. 288 (1993), 85-92; N. F. Cariello and T. R. Skopek, Mut. Res. 288 (1993), 103-112; P. M. Smooker and R. G. Cotton, Mut. Res. 288 (1993), 65-77). However, these methods are not suitable for a high sample throughput; nor do they exhibit the accuracy which is required for clinical diagnosis (E. P. Lessa and G. Applebaum, Mol. Ecol. 2 (1993), 119-129). Some biological methods (G. R. Taylor and J. Deeble, Genetic Analysis: Biomolecular engineering, 14 (1999), 181-186) have also been developed in addition to these chemical or physical methods. Many of these biological methods use the property possessed by proteins of the mutS family, i.e. that of binding selectively to mutation-determined base mispairings (P. Sachadyn et al., Nucl. Acids Res. 28 (2000) e36; A. Lishansky et al., Proc. Natl. Acad. Sci. USA 91 (1994), 2674-2678; WO 99/06591, U.S. Pat. No. 6,033,681, WO 99/41414, WO 99/39003 and WO 93/22462). However, because of their complexity, these methods have not so far gained acceptance in practice (G. R. Taylor and J. Deeble, Genetic Analysis: Biomolecular engineering, 14 (1999), 181-186). In the described methods, some of which were developed in the early 1980s, heteroduplexes are generated from the strand of a DNA of known sequence and from the complementary strand having an unknown sequence (e.g. A. L. Lu et al., Proc. Natl. Acad. Sci. USA 80, p4639-4643, 1983). If the unknown sequence possesses a mutation as compared with the complementary known sequence, the resulting base mispairings can be bound by repair proteins such as mutS, thereby making it possible to detect the mutation (S. S. Su and P. Modrich, Proc. Natl. Acad. Sci. USA 83, p 5057-5061, 1986). The complex which is formed in this way can be detected directly (e.g. WO 95/12688), indirectly (e.g. WO 93/02216) or by an additional enzymic treatment (e.g. WO 95/29258), with it also being possible for the mutS protein to be present in immobilized form (WO 95/12689).

In all the previously published methods, the DNA heteroduplexes ar produced by passive hybridization in a suitable buffer system (e.g. C. Bellanne-Chantelot et al., Mutation Research 382, 35-43, (1997)). In order to increase sample throughput, the heteroduplexes of several genes, but not of several individuals, can be produced by passive hybridization on an array (WO 99/06591). However, when several sequences are hybridized passively, more or less pronounced cross hybridizations occur. This inevitably leads to the formation of base mispairings, which are then bound by mutS without either of the participating sequences possessing a mutation. This results in a high background, with mutations being “covered up”. So far, this problem has only been partially solved by using single strand-binding (SSB) protein (Gotoh et al., Genetic Analysis 14, 47-50 (1997)). Furthermore, it is not possible to use the conventional passive arrays to examine an individual gene sequence from several individuals in parallel for mutations, as would be relevant for pharmacogenomic investigations. In addition, a further serious disadvantage of the mutS technology, in conjunction with passive DNA arrays, is the long duration of the hybridization, i.e. of up to 14 hours (WO 99/06591).

Consequently, no methods are known which are suitable for identifying SNPs, in particular unknown SNPs, in a highly parallelized sample throughput. In particular, no method is known which can be employed for rapidly identifying unknown SNPs using DNA chip technology. However, on account of its high sample throughput, such a detection system would be particularly desirable for the routine examination of test subjects participating in a clinical trial and the assignment, associated therewith, of a genotype to drug intolerance or to drug inactivity. An even higher sample throughput is required when medicating a large group of patients with active compounds where side effects or therapy failure frequently occur, as, for example, when treating breast cancer with antiestrogens.

Finally, it would be advantageous to have a method which can be used for simultaneously identifying previously unknown SNPs in a DNA sample in conjunction with analyzing the strength with which genes are expressed. This is particularly advantageous for individual investigations such as target validation and patient screening.

Consequently, the invention is based on the object of making available a method for detecting mutations in nucleotide sequences, which method permits a high sample throughput in a short time and with a high degree of reliability.

It was surprisingly possible to provide such a method in the form of an array, with it being possible to parallelize the hybridization reaction.

The object is achieved by means of a method for detecting mutations in nucleotide sequences, in which method single-stranded sample nucleotide sequences are hybridized with single-stranded reference nucleotide sequences, with the single-stranded reference nucleotide sequences or single-stranded sample nucleotide sequences being fixed before or during the hybridization, or heteroduplexes consisting of reference and sample nucleotide sequences being fixed after or during the hybridization, on a support in a site-resolved manner, and the incubation with a substrate which recognizes heteroduplex mispairings then taking place, in association with which the substrate binding can be detected.

In a preferred method for detecting mutations in nucleotide sequences,

a) a defined, single-stranded nucleotide sequence is loaded onto a nucleotide chip,

b) the nucleotide sequence which is to be examined for mutations, and which is complementary to the known nucleotide sequence, is likewise loaded onto the chip and a heteroduplex is prepared by hybridizing the two sequences,

c) the heteroduplex is then incubated with a substrate which recognizes mispairings, preferably a labeled substrate, and

d) the mispairings are detected by detecting the substrate which is attached to them.

Methods in which any single-stranded nucleotide sequences which are fixed on the support are degraded, after the hybridization, by adding a nuclease, preferably a mung bean nuclease or S1 nuclease, have proved to be particularly reliable and consequently particularly suitable. This is particularly surprising since, for example, the addition of SSB, as a protein binding single-stranded nucleic acids, after the hybridization has little effect on the binding of substrates which recognize mispairings.

It was surprisingly possible to provide methods which were particularly suitable as regards increasing sample throughput on the bases of an electronically addressable surface in combination with substrates which recognize mispairings, with it being possible to find mutation-specific mispairings reliably and considerably more rapidly than when using conventional passive hybridization techniques.

In this connection, the fixing of the single-stranded or double-stranded nucleotide sequences, and the hybridization, can be electronically controlled, in particular electronically accelerated.

A particularly preferred embodiment of the claimed method is characterized by a site-resolved, electronically accelerated hybridization, with the hybridization conditions, such as the current strength applied, the voltage applied or the duration of the electronic addressing, being set individually at the respective site. At the same time as, or after, the hybridization, the base mispairing can be detected by adding a substrate which recognizes mispairings.

These methods can be used to identify known and unknown point mutations, and also insertion and deletion mutations, rapidly and in an uncomplicated manner. If mispairings occur between the fixed nucleotide sequence and the nucleotide sequence to be examined, these are then recognized, for example, using labeled base mispairing-binding proteins or using electronic detection. It is consequently possible to pick out the mispairings on the chip. The SNPs are examples of detectable mispairings. In particular, when using the described methods, it is possible to examine several individuals in parallel for mutations on one chip.

The following term definitions are introduced for the further description of the invention:

In connection with the description of the detection method according to the invention, the expression “nucleotide sequence” is used for RNA or chemically modified polynucleotides as well as for deoxyribonucleic acid, with cDNA also being included within the term deoxyribonucleic acid;

The expression “reference nucleotide sequence” denotes a nucleotide sequence sequence, preferably a DNA sequence, which is used as a comparison sequence;

A “sample nucleotide sequence” is a labeled nucleotide sequence, preferably a DNA sequence, which is to be examined for mutations;

A “nucleotide chip” is characterized by a chip surface which is divided into zones to which the sample, or preferably reference, nucleotide sequences are in each case applied;

“Gene expression” is the transfer of hereditary information into RNA or protein.

The electronic addressing is effected by applying an electric field, preferably between 1.5 V and 2.5 V in association with an addressing duration of between 1 and 3 minutes. Due to the electric charge on the nucleotide sequences to be addressed, their migration is greatly accelerated by an electric field being applied. In this connection, the addressing can be effected in a site-resolved manner; in this case, addressing takes place consecutively to different zones on the chip surface. At the same time, different addressing and hybridization conditions can be set at the individual sites.

When carrying out the detection method according to the invention, nucleotide sequence heteroduplexes consisting of a predetermined nucleotide sequence, i.e. the reference nucleotide sequence, and of the complementary nucleotide sequence from a physiological sample, i.e. the sample nucleotide sequence, are initially produced on a chip surface using electronic addressing. The mispairings which ar formed in this connection indicate an SNP in the sample nucleotide sequence and can be detected using a substrate which binds to the mispairing site. Proteins which bind base mispairings are suitable for this purpose. Base mispairing-binding proteins can, for example, be mutS or mutY, preferably derived from E.coli, T. therinophilus or T.aquaticus, MSH 1 to 6, preferably derived from S.cerevisiae, S1 nuclease, T4 endonuclease, thymine glycosylase, cleavase or fusion proteins which contain a domain from these base mispairing-binding proteins. However, other proteins or substrates can also be used for this purpose if they are able to specifically recognize a base mispairing in a nucleotide sequence double strand and to bind to it.

In the method according to the invention, the reference nucleotide sequence, for example, can be employed as a biotinylated oligonucleotide which is either synthesized or prepared by amplification using sequence-specific oligonucleotides, one of which is biotinylated at the 5′ end. After that, the reference nucleotide sequence is converted into the single-stranded state by melting, preferably in a buffer solution having a low salt content, and applied to a predetermined position on a chip by means of electronic addressing. Examples of suitable chips are those marketed by Nanogen (San Diego/USA). The reference nucleotide sequence can be applied, for example, using a Nanogen molecular biology workstation, preferably using the parameters specified by the manufacturer. Unless otherwise indicated, Nanogen's chips and/or their molecular biology workstation is/are used in accordance with the manual which is supplied with them; the method of use is also described in Radtkey et al., Nucl. Acids Res. 28, 2000, e17.

The sample nucleotide sequence, which is complementary to the sequence which has already been applied to the chip, can now be loaded onto the chip which has been prepared in this way. For this purpose, dye-labeled oligonucleotides are synthesized or generated by amplifying using sequence-specific oligonucleotides one of which is dye-labeled at the 5′ end. In this connection, the dye-labeled nucleotide in the sample nucleotide sequence constitutes the complementary counterstrand to the biotinylated strand of the reference nucleotide sequence. The sample nucleotide sequence has also to be converted beforehand into the singl-stranded state by being melted, for xample in a buffer solution having a low salt content, and then applied to the biotinylated reference nucleotide sequence by means of electronic addressing. This results in the formation, by hybridization, of a nucleotide sequence heteroduplex consisting of the reference nucleotide sequence and the sample nucleotide sequence. The heteroduplex can also be prepared on an electronically addressable surface, for example using a Nanogen molecular biology workstation and employing the parameters specified by the manufacturer. Successful hybridization can be monitored optically, and at the same time determined quantitatively, by detecting the dye which is coupled to the heteroduplex.

Alternatively, the sample nucleotide sequence can also be biotinylated and electronically addressed, as just described. It is also possible to hybridize in solution, with subsequent electronic addressing and with one of the two nucleotide sequences of the heteroduplex being biotinylated. Apart from derivatizing with biotin, it is also possible to use other molecular groups, which bind to an electronically addressable surface, for fixing nucleotide sequences. Thus, it is likewise possible, for example, to effect the fixing using introduced thiol groups, hydrazine groups or aldehyde groups.

If the sample nucleotide sequence now exhibits a mutation as compared with the reference nucleotide sequence, there will then be a mispairing in the heteroduplex. Preference is given to using proteins of the mutS family, which proteins recognize these mispairings with a high degree of specificity, for identifying such mispairings. The mispairing-recognizing mutS proteins derived from E.coli and from T. thermophilus, and also mutS fusion proteins, such as MBP-mutS, are particularly suitable for this purpose. The mispairing-recognizing substrate is preferably added in excess, with it being possible to remove unbound substrate by washing.

In addition to dye-carrying, luminescent and fluorescent groups, the mispairing-recognizing protein can also contain polymeric labels (J. Biotechnol. 35, 165-189, 1994), metal labels, enzymic or radioactive labeling or quantum dots (Science Vol 281, 2016, 25 Sep. 1998). In this connection, the enzyme labeling can, for example, be a direct enzyme coupling or an enzyme substrate transfer or an enzyme complementation. Chloramphenicol acetyltransferase, alkaline phosphatase, luciferase and p roxidase are particularly suitable for the enzymic labeling.

Substrate labeling using dyes which absorb or emit light in the range between 400 and 800 nm is particularly preferred. The fluorescent dyes which are suitable for the labeling and which are to be preferred are particularly Cy™3, Cy™5 (from Amersham Pharmacia), Oregon Green 488, Alexa Fluor 488, Alexa Fluor 532, Alexa Fluor 546, Alexa Fluor 594, Alexa Fluor 647, Bodipy 558/568, Bodipy 650/665, Bodipy 564/570 (e.g. from Mobitec, Germany), S 0535, S 0536 (e.g. from FEW, Germany), Dy-630-NHS, Dy-635-NHS, EVOblue30-NHS (e.g. from Dynomics, Germany), FAR-Blue, FAR-Fuchsia (e.g. from Medway, Switzerland), Atto 650 (from Atto Tech, Germany), FITC and Texas Red. In addition to the directly labeled substrates, it is also possible to use labeled antibodies which are directly directed against mutS or against a fused peptide domain, such as MBP.

If a dye-labeled mutS protein, for example, is now incubated with the heteroduplex nucleotide sequence which is bound on the chip surface, the protein then binds preferentially at the positions on the chip where mispairings have been formed within the heteroduplex. The bound dye-labeled mutS proteins can then be quantitatively determined using optical sensors, for example using the Nanogen molecular biology workstation in combination with suitable analytical software.

Alternatively, the binding of the substrate which recognizes mispairlngs can also be effected using electrical methods such as cyclovoltametry or impedance spectrometry (e.g. described in WO 97/34140). These electrical methods for reading a nucleotide chip are characterized, in particular, by the fact that there is no need to use mispairing-recognizing substrates which are labeled. The electrical detection methods are also suitable for detecting formation of the heteroduplex. Thus, it can be advantageous to combine electrical and optical methods for monitoring individual procedural steps. Alternative methods for detecting a substrate which recognizes mispairings are measurement of the surface plasmon resonance (e.g. in J. Pharm. Biomed. Anal. 22(6), 1037-1045, 2000), the cantilever technique (e.g. described in Nature 1995 June 15, 375(6532), 532 or in Biophysical Journal, 1999 June, 76(6), 2922-33) or the Microcantilever technique (e. g. described in Science 288, 316-318, 2000) or detection using acoustic methods (as described, for example, in WO 97/43631) or using gravimetric methods.

The method according to the invention is not only suitable for detecting gene mutations; it can also indicate differences in the level of expression of the mRNA which is expressed in various cells or tissues. For this, the mRNA is converted, in a preferred embodiment, into cDNA, with the resulting cDNA being used for the measurement. The detection is preferably effected by means of a dye which is coupled to the sample nucleotide sequence and which is detected optically. Since the quantity of the dye which is present at a given chip position correlates with the quantity of the mRNA or cDNA, analyzing the dye intensity at several chip positions makes it possible to determine differences in the expression level in various cells or tissues. At the same time, the level of expression of a gene in different samples, or of different genes, can be determined in parallel. The parallel detection of mutations and differences in gene expression in the same sample not only saves time but is also less susceptible to error because of the samples being treated uniformly in the two detection systems.

The possibility of determining gene expression and simultaneously detecting mutations in parallel, in an integrated manner in one procedure, constitutes another important advantage of the present method.

Apart from, for example, optically detecting fluorescence-labeled mutS which is specifically bound to mispairings, the substrate binding can also be detected using electrical methods. Impedance spectroscopy is particularly suitable for this purpose, with the change in the alternating current resistance at the site of measurement, which change depends on the quantity of substrate bound, being determined. However, it is also possible to conceive of using cyclovoltametry to measure the potential difference between an electron donor or acceptor which is bound to the nucleotide sequences and an electrically conductive surface, with the electron flow being altered by the binding of the substrate.

In a preferred embodiment of the method according to the invention, the electronic addressing takes place on a chip surface which is coated with a permeation layer. The permeation layer enables small ions to flow to the electrically conductive surface of the chip, resulting in the circuit being closed, without the nucleotide sequences or the substrate coming into contact with the chip surface and there themselves being oxidized or reduced. Suitable permeation layers which are preferred are nonionic polymeric or gelatinous materials which possess a high permeability for nucleotide sequences and the substrate employed such that good penetration of the permeation layer is achieved when electronically addressing with the nucleotide sequences or when incubating with the substrate which recognizes mispairings. Thus, when mutS is used as the substrate, for example, it is preferable to coat the electronically addressable chip with hydrogel rather than agarose. Thus, as compared with the agarose chip, the hydrogel chip offers the advantages of higher sensitivity and better discrimination between mispaired and perfectly paired DNA. This is surprising insofar as it was not possible to predict that the constitution of the permeation layer would have such a great influence on the sensitivity of the detection.

Furthermore, low salt conditions have proved to be advantageous when implementing the method. Surprisingly, the ability of the mutS substrate to bind to base mispairings is not adversely affected by the low salt conditions. Based on using mutS as the substrate, the mispairing binding should take place at a salt concentration of from 10 to 300 mM, preferably of from 10 to 150 mM; a salt concentration of from 25 to 75 mM has proved to be particularly preferable. The optimum salt concentrations for mispairing recogniuon by other substrates can readily be ascertained in analogy with the implementation example. Furthermore, the penetration of the permeation layer by the mispairing-recognizing substrate can be increased by adding detergents, such as Tween-20. Surprisingly, mutS does not lose its ability to bind to mispairings when detergents are added, either.

In another preferred embodiment, the measurement accuracy of the method is increased by adding substances, such as BSA, which block nonspecific binding sites. In addition to this, th addition of SSB can have a positive effect on measurement accuracy provided that single-stranded nucleic acid fragments are bound by the mispairing-recognizing susbtrate mployed, as is the case, for example, with mutS. However, it was only possible to achieve a small improvement in measurement accuracy when mutS was used as a substrate.

It is all the more surprising that degrading single-stranded nucleotide sequences, after the electronic hybridization and before adding the mispairing-recognizing substrate, results in a substantial increase in the measurement accuracy. This effect can be exploited both in association with electronically addressable chips and in association with a procedural arrangement using passive hybridization on an array surface. In this connection, the single-stranded nucleotide sequences can be degraded enzymically using nucleases, such as mung bean nuclease or the S1 nuclease. The reliability of the measurement is substantially increased by introducing such a nuclease digestion into the assay.

A problem associated with detecting different mutations in parallel, i.e. the mispairings M, AG, AC, GG, GT, CT, CC and TT, and the mispairings due to deletion or insertion of individual nucleotides, is that the mispairing-recognizing substrates recognize some mutations better than others. Thus, mutS, for example, recognizes the mispairings GT, GG and AA better than it recognizes the mispairings TT, CC and AC.

In this regard, it is to be noted that an important mechanism leading to the genesis of base exchange mutations is the deamination of 5-methylcytosine. In mammals, about 3-5% of all cytosine residues are methylated (this modification contributes to the inactivation of genes), and the base thymine is formed when such a methyl cytosine spontaneously deaminates. Although there are special repair enzymes which recognize, and repair, the resulting GT mispairing in the DNA double strand, the mutation nevertheless remains unrecognized in some cases and then leads, at the next DNA replication cycle, to a conversion of the original CG basepair into a TA basepair. The importance of this mechanism is made clear by the fact that almost a third (31.7%) of all point mutations which have been found in genetically determined diseases in humans have arisen as a result of the deamination of 5-methylcytosine (Ramsahoye et al., Blood Reviews (1996) 10, 249-261).

The method described here specifically detects this very frequently occurring base exchange mutation particularly well.

A particular advantage of using mutS as a substrate is that all the mispairings which mutS is less able to recognize can be converted into their corresponding mispairings which mutS recognizes particularly well.

If a DNA strand in which a cytosine residue has been mutated to thymine is hybridized, for example on an electronically addressable chip, with an unmutated reference counterstrand, this then results in a GT mispairing which can be reliably detected using the E.coli mutS protein. However, in addition, it is also possible to use the method which has been introduced here for detecting other mutations, in particular those point mutations which lead, when the mutated DNA is hybridized with an unmutated counterstrand, to a G:G, C:T or A:A mispairing. mutS can likewise be used to detect insertions or deletions of one or two bases. Furthermore, the proportion of mutations which can be uncovered using the method which is described here can be increased by hybridizing both strands of a DNA to be tested with the respective reference counterstrand. This can be illustrated by the following example: provided it is not prepared, a base exchange in which a guanine is replaced by a cytosine leads to the conversion of the original G:C basepair into a C:G basepair.

| | | |
|---|---|---|
| Reference DNA | Strand (a) | 5′-...ATGTA...-3′ |
| (wild type): | | |
| | Counterstrand | 3′-...TACAT...-5′ |
| | (b) | |
| DNA to be tested | Strand (aₘᵤₜ) | 5′-...ATCTA...-3′ |
| (mutated): | Counterstrand | 3′-...TAGAT...-5′ |
| | (bₘᵤₜ) | |

If strand (aₘᵤₜ) of the DNA to be tested is now hybridized with the counterstrand (b) of the reference DNA, this then results in a CC mispairing, which is only weakly bound by mutS. On the other hand, when the mutated counterstrand (bₘᵤₜ) hybridizes with the reference strand (a), this then results in the formation of the corresponding GG mispairing, which mutS can detect much more readily. This situation is similar in the case of point mutations in which an adenine has been replaced by thymine. If both strands of such a mutated DNA are hybridized with what are in each case the complementary, unmutated reference strands, this then results, on the one hand, in a TT mispairing, which is only weakly bound by mutS, and, on the other hand, in a corresponding AA mispairing, which mutS is better able to recognize. Similarly, an AC mispairing can be replaced by the corresponding TG mispairing.

When corresponding base mispairings are used, either both strands of a nucleotide sequence can be hybridized electronically at separate sites or a mixture of the two single strands is fixed on a chip surface.

T.thermnophilus mutS has surprisingly proved to be particularly suitable for detecting insertions or deletions of individual nucleotides, preferably of from one to three nucleotides. Thus, it is also possible to adapt the method to the given requirements by combining individual mispairing-recognizing substrates.

The electronic addressing can be effected, for example, on a chip, on which the nucleotide sequences A, B, C . . . , N are already fixed at sites a, b, c to n, using a mixture containing nucleotide sequences from the group A′, B′, C′, . . . , N′. In this case, the nucleotide sequences A/A′ to N/N′ in each case constitute a reference and sample nucleotide sequence pair. After the electronically accelerated hybridization, the stringency of the hybridization conditions can be increased, for example, by reversing the polarity of the electrical field. This can be effected in a site-resolved manner and consequently be adjusted individually in the case of each site.

In a particularly preferred embodiment, the electronic addressing on the chip surface is effected in a controlled and consecutive manner. If identical or different reference nucleotide sequences are fixed on an electronically addressable chip in a site-resolved manner, the electronically accelerated hybridization with the given sample nucleotide sequence to be tested is then effected site-specifically and consecutively. If, for example, different reference nucleotide sequences A, B, C, . . . , N are attached at sites a, b, c, . . . , n, hybridization with the samples A′, B′, C′, . . . , N′ is then effected consecutively and site-specifically such that the heteroduplex AA′ can be formed at site a, the heteroduplex BB′ at site b, the heteroduplex CC′ at site c up to the heteroduplex NN′ at site n. Alternatively, the sample nucleotide sequences can, of course, also be attached to the chip surface and electronically accelerated hybridization is then effected consecutively with the respective reference nucleotide sequences. The hybridization of sample nucleotide sequences of differing origin, for example derived from different patients, with what is always the same reference nucleotide sequence is also preferably carried out using the above-described procedural scheme. This embodiment of the method according to the invention is characterized by a high degree of reliability. However, the arrangement of the measurements as a consecutive process only becomes possible by using an electronically addressable surface. As a result, the method according to the invention can be carried out in a highly parallelized manner on an electronically addressable surface; this makes it possible to achieve high sample throughput. In this case, too, there is the possibility of varying the hybridization conditions by reversing the polarity of the electrical field. Because of the long duration of the hybridization process, which as a rule amounts to several hours, and because of the fact that the inaccuracy of the hybridization is too high, passive hybridization methods are not suitable for such a course of action.

Such a method is not only considerably more reliable for finding mutations than are the passive hybridization techniques which are known from the prior art, but also considerably faster. Thus, a chip having a 10×10 array surface, on which 100 parallel measurements can be carried out in a site-resolved manner, is read in from 4 to 8 hours when the last method to be described is used. In a passive method, it would be necessary to carry out 100 different hybridization assays, each individual one of which would last approx. 14 hours (as described, for example, in WO 99/06591). Such a method is therefore scarcely practicable.

Another advantage of the claimed methods is that, in addition to being able to qualitatively detect the presence of a mutation, it is also possible to quantitatively determine the transcription of the mutated nucleic acid sequence. This is of interest, for example, when analyzing heterozygous genotypes. In this connection, the quantity of bound substrate which specifically recognizes mispairings is, to a first approximation, a measure of the rate at which the mutated nucleotide sequence is transcribed. A standardization is helpful, particularly when quantitatively determining mutated nucleotide sequences. For this, the reference or sample nucleotide sequence, for example, can be labeled with a dye. In this way, it can be checked optically that the same quantity of nucleotide sequences is fixed at the site of the standard measurement as at the site of the actual measurement. A completely complementary nucleotide sequence is then added in excess at the site of the standard measurement such that all the fixed nucleotide sequences are hybridized without there being any mispairings. Depending on the procedural arrangement, further additives, such as BSA, SSB, detergents, etc., are then added. After the substrate which recognizes mispairings has been added, a comparison value can then be determined, with this value serving as standard. The mutated nucleotide sequence is quantitatively determined in parallel, with the mispairing-recognizing substrate likewise being added. The difference between the standard value and the experimental value makes it possible to provide a quantitative assessment of the rate at which the mutated nucleotide sequence is transcribed. In order to make the quantitative determination more precise, it is appropriate to construct a calibration curve using different concentrations of the mutated nucleotide sequence since, for example, the increase in the binding of mutS to the heteroduplex with the number of base mispairings which occur is not linear but, instead, flattens off slightly. A reason for this could be mass transfer effects at the site of measurement.

A further advantage of the present method follows from this. Since substrate binding increases rapidly when mispairings are infrequent, the measurement is very sensitive; when a large number of mispairings are present, the substrate binding increases more slowly resulting in a large measurement range being achieved. Thus, solutions of the individual nucleotide sequences having a concentration of from 100 pM to 100 μM, preferably of from 1 nM to 1 μM, are preferably used for the electronic addressing. In this range, there is no difficulty in quantitatively determining the heteroduplexes which are carrying the mispairings which have been generated.

If the method which is described here is to be carried out using DNA which has been amplified from patient samples, the quantity of DNA which can be obtained from this source is then as a rul limited. This is because too powerful an amplification would lead to the accumulation of mutat d strands, on account of the error rate of the polymerase, and thus lead to an increase in the background. In addition to this, when patient DNA is used, variations in the concentration of the DNA between different patient samples are to be expected. These variations could give rise to variations in the mutS signal and, in the extreme case, could prevent the mutation being detected. It has been found, surprisingly, that, particularly when mutS is used as the substrate which recognizes mispairings, the method according to the invention is suitable for reliably and quantitatively recognizing mutations even when the DNA concentrations are low and/or varying. This is due to the fact that relatively large variations in the concentration of the DNA employed do not lead to similarly large variations in the binding of mutS. Furthermore, the method according to the invention exhibits a high degree of reliability in the detection of mutations, in particular in the range of DNA concentrations which are relevant in practice, i.e. as are obtained when investigating samples derived from patients. Furthermore, the claimed method surprisingly exhibits a high degree of invulnerability toward variations in the quantity of nucleotide sequence prepared. Consequently, it is possible to compare different patient samples even when the individual samples do not have precisely the same concentration of DNA.

The methods according to the invention are consequently suitable for rapidly and reliably detecting mutations. Thus, a large number of samples can be examined in parallel. This thereby improves genotypic screening for previously unknown mutations. Thus, large quantities of human genome sequence data have become available, for example, during the course of the human genome project, with it being possible to use these data to construct electronically addressable chips which can be tested against the nucleotide sequences of samples obtained from different individuals. This approach can be used to rapidly identify a large number of mutations which do not necessarily have to be expressed phenotypically.

In an analogous manner, it is possible to examine samples which have been obtained from the cells of a particular organism for the presence of a mutation which is inherited dominantly or recessively. In this connection, the possibility of the large sample throughput enables a large group of people, for example newborn babies, to be screened for the presence of mutations in particular genes. This facilitates the early recognition of a disease disposition and the early treatment of inherited genetic defects, such as cystic fibrosis, Huntington's chorea or sickle cell anemia, all of which are due to specific known mutations. Similarly, it is also possible to use such a method to investigate any possible intolerance to a drug or the inactivity of a drug, such as resistance to tamoxifen, in a patient, as long as the intolerance correlates with a known mutation or it is the analysis itself which is able to produce a correlation.

On account of the high speed of the method, and on account of the high degree of parallelization which can be achieved, it is possible, using high sample throughput, to investigate many different samples from patients who are suffering from a hereditary disease. This facilitates the task of achieving a correlation between a clinical syndrome and particular mutations. In addition to this, it is possible to screen more efficiently for mutations which have been acquired during the course of life and which can be correlated with particular diseases. Thus, it is possible, for example, to detect a mutation in the DNA-binding domain of the antioncogene p53 (exon 8) in different tumor samples rapidly and without difficulty.

In addition, it should be pointed out that many different assays can be developed, depending on the choice of the reference nucleotide sequences, with it being possible to use the claimed methods to carry out these assays rapidly and reliably. Thus, individual exons of a gene can, for example, be used as reference nucleotide sequences independently of each other. This makes it possible not only to demonstrate that a mutation is present but also where such a mutation is located. By choosing suitable gene fragments as reference nucleotide sequences, the site of the mutation can even be determined precisely if use is made of fragments which are in each case displaced by one nucleotide on the basis of the whole sequence being examined. In particular, the separate use of gene regions encoding individual protein domains offers many different possibilities of answering a variety of questions. Thus, it is by now frequently possible to assign, to individual protein domains, particular biological functions within a protein, such as an enzymic activity, a binding site having a regulatory effect, or the ability to become incorporated into a cell membrane. If the individual nucleic acid segments encoding these domains are fixed separately on the chip surface, a mutation can then be correlated directly with the change in a particular protein property. This approach is particularly suitable for investigating metabolic pathways in which several proteins are involved.

In addition to the method according to the invention, the present invention also relates to an assay pack in the form of a kit. This kit contains an electronically addressable chip, reference nucleotide sequences, which can be present in free form or already fixed on the chip surface, and at least one substrate which specifically recognizes mispairings. The reference nucleotide sequences which are included must in each case be appropriate for the intended purpose of the assay. Preference is given to using E.coli mutS as the mispairing-recognizing substrate. However, for special problems, it is also possible to include other substrates in the kit, such as T.thermnophilus mutS for detecting nucleotide insertions or deletions.

Proteins which are directly labeled with a dye and which recognize mispairings, in particular labeled mutS, have not previously been described. It was surprisingly possible to prepare such a directly labeled substrate without any loss of binding specificity.

Consequently, the present invention also relates to a method for preparing dye-labeled proteins which recognize mispairings, with an ester, preferably a succinimidyl ester, of the dye being reacted, at low concentration, preferably between 1 μM and 100 μM, under mild conditions and with the exclusion of light, with a protein which recognizes mispairings, preferably mutY, MSH1 to MSH6, S1 nuclease, T4 endonuclease, thymine glycolase or cleavase and, particularly preferably, with mutS. In addition, use of a HEPES buffer consisting of from 5 mM to 50 mM N-2-hydroxyethylpiperazine-N′-2-ethanesulfonic acid (HEPES), pH 7.5 to 8.5, 50 to 500 mM KCl, 1 to 15 mM MgCl₂, 5 to 15% glycerol in distilled water, has proved to be advantageous.

Using this method, it is possible to label mispairing-recognizing proteins directly with dyes without the proteins losing their specific binding activity. Consequently, the present invention furthermore relates to mispairing-recognizing proteins, preferably mutY, MSH1 to MSH6, S1 nuclease, T4 endonuclease, thymine glycolase or cleavase and, particularly preferably mutS, which are dye-labeled. In this connection, dyes which are particularly suitable for the labeling are Cy™3, Cy™5, Oregon Green 488, Alexa Fluor 488, Alexa Fluor 532, Alexa Fluor 546, Alexa Fluor 594, Alexa Fluor 647, Bodipy 558/568, Bodipy 650/665, Bodipy 564/570, S 0535, S 0536, Dy-630-NHS, Dy-635-NHS, EVOblue30-NHS, FAR-Blue, FAR-Fuchsia, Atto 650, FITC and Texas Red.

In the same way, the invention relates to fusion proteins which recognize mispairings and which can be labeled, for example, with an antibody-binding epitope, such as MBP, or with an enzymic group, preferably with chloramphenicol acetyltransferase, alkaline phosphatase, luciferase or peroxidase. However, the label can also be a luminescent or radioactive group.

The present invention also relates to the use of mutS for a method for detecting mutations, in a site-resolved manner, in nucleotide sequences on a support, preferably on an electronically addressable surface. In this connection, it is particularly advantageous to use mutS which is directly fluorescence-labeled.

### IMPLEMENTATION EXAMPLES

### General Comments

Proteins of the mutS family which are known to play an important role in the recognition and repair of DNA damage in eukaryotes, bacteria and Archeae (R. Fishel, Genes Dev. 12 (1998), 2096-2101) are used as base mispairing-binding proteins. These proteins bind specifically to segments of the DNA which contain base mispairings and initiate repair of the damage by recruiting enzymes.

On account of their special binding properties, these proteins can be used for detecting mutations (G. R. Taylor and J. Deeble, Gen tic Analysis: Biomolecular engineering, 14 (1999), 181-186).

In the examples which are given, mutations are detected by means of the electronically accelerated hybridization of the reference DNA with the DNA to be tested, taken in combination with novel, dye-labeled mutS proteins. A molecular biology workstation from Nanogen is used for this purpose. Unless otherwise described in the implementation examples, the measurements are performed in accordance with the manufacturer's instructions (manual for Nanogen's molecular biology workstation). A description of the measurement method is also to be found, for example, in Radtkey et al., Nucl. Acids Res. 28, 2000, e17.

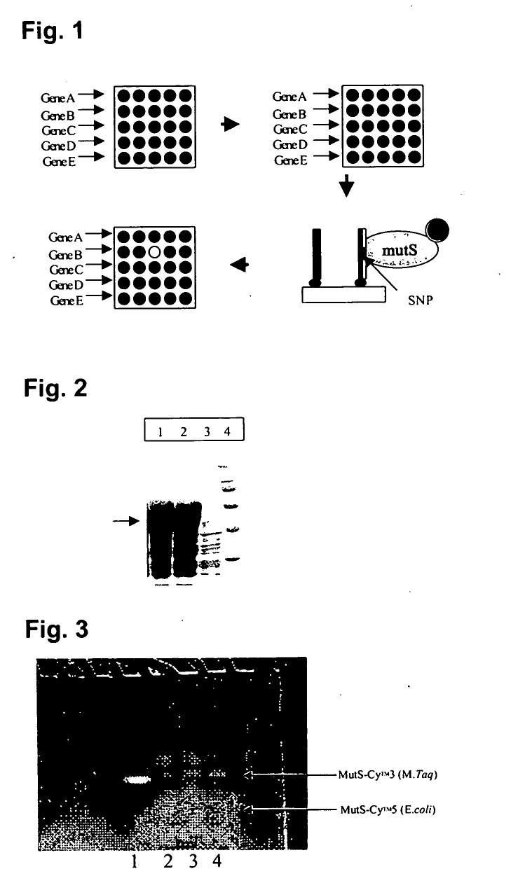

FIG. 1 shows a diagram of the parallel detection of mutations and illustrates the following:

(1) fragments of the genes A-E are electronically addressed, as single-stranded reference DNA, to individual positions on a Nanogen™ chip (Nanogen Inc., San Diego, USA), and

(2) hybridized with the dye-labeled test DNA in an electronically accelerated and site-resolved manner.

(3) Base mispairings in the resulting heteroduplexes reflect a mutation in the test DNA as compared with the reference DNA and can be located, for example, using a dye-labeled mutS protein.

(4) Optical analysis of the chip subsequently enables a mutation to be assigned to a gene fragment.

As indicated in each case, the following examples have made use of E.coli mutS, T. thertnophilus mutS, T.aquaticus mutS or the fusion protein MBP-mutS. After the functional activity of the labeled mutS protein had been checked, the resulting dye-labeled mutS protein was used, in a subsequent step, for detecting mutations in electronically addressed DNA heteroduplexes.

The nucleotide sequences which were used for constructing the nucleotide chips are depicted, together with their respective labels, in the following table.

| | | |
|---|---|---|
| Seq. ID No. | Name | Sequence |

| | | |
|---|---|---|
| 6 | AT | 5′-Cy3-tgg cta gag atg atc cgc act tta act tcc gta tgc-3′ |
| 7 | GT | 5′-Cy3-tgg cta gag atg atc cgc gct tta act tcc gta tgc-3′ |
| 10 | sense | 5′-Biotin-aag cat acg gaa gtt aaa gtg cgg atc atc tct agc ca-3′ |
| 11 | sense | 5′-Biotin-aag cat acg gaa gtt aaa gtg cgg atc atc tct agc-3′ |
| 12 | AT | 5′-Cy3-tgg cta gag atg atc cgc act tta act tcc gta tgc-3′ |
| 13 | GT | 5′-Cy3-tgg cta gag atg atc cgc gct tta act tcc gta tgc-3′ |
| 14 | AA | 5′-Cy3-tgg cta gag atg atc cgc aca tta act tcc gta tgc-3′ |
| 15 | AG | 5′-Cy3-tgg cta gag atg atc cgc aat tta act tcc gta tgc-3′ |
| 16 | CA | 5′-Cy3-tgg cta gag atg atc cgc acc tta act tcc gta tgc-3′ |
| 17 | CC | 5′-Cy3-tgg cta gag atg atc ccc act tta act tcc gta tgc-3′ |
| 18 | CT | 5′-Cy3-tgg cta gag atg atc cgc cct tta act tcc gta tgc-3′ |
| 19 | GG | 5′-Cy3-tgg cta gag atg atc cgc agt tta act tcc gta tgc-3′ |
| 20 | TT | 5′-Cy3-tgg cta gag atg atc cgc tct tta act tcc gta tgc-3′ |
| 21 | ins + 1T | 5′-Cy3-tgg cta gag atg atc cgc act ttt aac ttc cgt atg c-3′ |
| 22 | ins + 2T | 5′-Cy3-tgg cta gag atg atc cgc act ttt taa ctt ccg tat gc-3′ |
| 23 | ins + 3T | 5′-Cy3-tgg cta gag atg atc cgc act ttt tta act tcc gta tgc-3′ |
| 24 | PC se | 5′-Biotin-aag atc ttc agc tga cct agt tcc aat ctt ttc ttt tat-3′ |
| 25 | PC AT | 5′-Cy3-aa ata aaa gaa aag att gga act agg tca gct gaa gat c-3′ |
| 26 | PC GT | 5′-Cy3-aa ata aaa gaa aag att gga gct agg tca gct gaa gat c-3′ |
| 27 | bcl se | 5′-Biotin-aag gtc gcg gga tgc ggc tgg atg ggg cgt gtg ccc ggg-3′ |
| 28 | bcl AT | 5′-Cy3-ag ccc ggg cac acg ccc cat cca gcc gca tcc cgc gac c-3′ |
| 29 | bcl GT | 5′-Cy3-ag ccc ggg cac acg ccc cat tca gcc gca tcc cgc gac c-3′ |
| 30 | Brc se | 5′-Biotin-a aat gtt att acg gct aat tgt gct cac tgt act tgg aa-3′ |
| 31 | Brc AT | 5′-Cy3-c att cca agt aca gtg agc aca att agc cgt aat aac at-3′ |
| 32 | Brc GT | 5′-Cy3-c aft cca agt aca gtg agc ata att agc cgt aat aac at-3′ |
| 33 | Met se | 5′-Biotin-a act ata gta ttc ttt atc ata cat gtc tct ggc aag ac-3′ |
| 34 | Met AT | 5′-Cy3-t ggt ctt gcc aga gac atg tat gat aaa gaa tac tat ag-3′ |
| 35 | Met GT | 5′-Cy3-t ggt ctt gcc aga gac atg tgt gat aaa gaa tac tat ag-3′ |
| 36 | MSH se | 5′-Biotin-a acc ttt ctc caa aat ggc tgg tcg tac ata tgg aac ag-3′ |
| 37 | MSH AT | 5′-Cy3-a cct gtt cca tat gta cga cca gcc att ttg gag aaa gg-3′ |
| 38 | MSH GT | 5′-Cy3-a cct gtt cca tat gta cga cta gcc att ttg gag aaa gg-3′ |
| 39 | p53 se | 5′-Biotin-aa agt tcc tgc atg ggc ggc atg aac cgg agg ccc atc-3′ |
| 40 | p53 AT | 5′-Cy3-ag gat ggg cct ccg gtt cat gcc gcc cat gca gga act-3′ |
| 41 | p53 GT | 5′-Cy3-ag gat ggg cct ccg gtt cat gct gcc cat gca gga act-3′ |
| 42 | Rb se | 5′-Biotin-a aat aag atc aaa taa agg tga atc tga gag cca tgc aa-3′ |
| 43 | Rb AT | 5′-Cy3-c ctt gca tgg ctc tca gat tca cct tta ttt gat ctt at-3′ |
| 44 | Rb GT | 5′-Cy3-c ctt gca tgg ctc tca gat tta cct tta ttt gat ctt at-3′ |

### Example: Cloning, Expression and Purification of E.coli mutS

The DNA sequence encoding E.coli mutS was amplified by PCR and isolated using standard methods. The 5′ primer (SEQ. ID No. 1) introduces a BamHI cleavage site directly upstream of the start codon while the 3′ primer (SEQ. ID No. 2) generates a HindIII cleavage site downstream of the stop codon. PCR is known to the skilled person and was carried out in accordance with the following scheme:

A toothpick tip of E.coli XL1 Blue (Stratagene, Amsterdam Zuidoost, The Netherlands) is added to a 100 μl PCR mixture containing 71 μl of H₂O and 10 μl of 10 μM 5′ primer, 10 μl of 10 μM 3′ primer, 10 μl of 10×PCR buffer containing MgSO₄ (Roche, Mannheim), 2 μl of DMSO, 1 μl of dNTP's (in each case 25 μM) and 2 μl of Pwo polymerase (=10 U). The PCR is run in accordance with the following program: 94° C. for 5 minutes with 30 subsequent cycles of 0.5 minutes at 94° C., 0.5 minutes at 55° C. and 2.5 minutes at 72° C. The end of the PCR is followed by an incubation at 72° C. for 7 minutes.

The mutS PCR product (SEQ. ID. No. 3) is purified on a 1% TAE agarose gel and the desired DNA is isolated from an excised agarose block using the gel extraction kit (Qiagen, Hilden, Germany).

The isolated DNA is quantified on a gel and cut with BamHI and HindIII. In a 60 μl mixture, 10 μl of mutS PCR product (about 2 μg) are combined with 30 U of BamHI (3 μl, NEB, Heidelberg), 30 U of HindIII (3 μl, NEB, Heidelberg), 6 μl of 10×NEB2 buffer (NEB, Heidelberg), 0.6 μl of 100×BSA (NEB, Heidelberg) and 37.4 μl of H₂O and the whole is incubated at 37° C. for 4 hours. The enzymes are subsequently inactivated at 70° C. for 10 minutes. After 6 μl of Na acetate, pH 4.9 and 165 μl of ethanol have been added, the DNA is precipitated overnight at 4° C. After the pellet has been washed in 70% ethanol, it is dried in air. The DNA is taken up in 30 μl of TE (10 mM trisHCl, 1 mM EDTA, pH8). The E.coli expression plasmid pQE30 (SEQ. ID No. 4) (Qiagen, Hilden) is likewise cut with BamHI and HindIII. In a 60 μl mixture, 10 μl of pQE30 are combined with 30 U of BamHI (3 μl, NEB, Heidelberg), 30 U of HindIII (3 μl, NEB, Heidelberg), 6 μl of 10×NEB2 buffer (NEB, Heidelberg), 0.6 μl of 100×BSA (NEB, Heidelberg) and 37.4 μl of water and the whole is incubated at 37° C. for 4 hours. The enzymes are subsequently inactivated at 70° C. for 10 minutes. After 6 μl of Na acetate, pH 4.9, and 165 μl of ethanol have been added, the DNA is precipitated overnight at 4° C. After the pellet has been washed with 70% ethanol, it is dried in air. The DNA is taken up in 30 μl of TE (10 mM trisHCl, 1 mM EDTA, pH8).

The quantities of pQE30 and mutS are compared on an agarose gel, and 100 ng of plasmid (2 μl) and 150 ng (5 μl) of mutS DNA are combined, in a 20 μl ligation mixture, with 2 μl of 10× ligase buffer (Roche, Mannheim), 2 μl of ligase (2 U, Roche, Mannheim) and 9 μl of H₂O, and the whole is incubated at 37° C. for 2 hours. The ligation mixture is subsequently transformed into E.coli TOP10 (from Stratagene, La Jolla, San Diego, USA) using the CaCl₂ method (Ausubel et al., Current protocols in molecular biology, Vol. 1, ED Wiley and Sons, 2000). The cells are selected for resistance to ampicillin and the plasmid content of positive clones is investigated by means of miniprep analysis. Protein induction was performed on clones in which the desired pQE30-mutS (SEQ. ID. No. 5) plasmid was found.

A 5 ml LB (containing 100 μg of ampicillin/ml) overnight culture of E.coli TOP10 harboring the plasmid pQE30-mutS is diluted such that an OD₅₉₅ of 0.05 is obtained in a subsequent 100 ml LB culture (100 μg of ampicillin/ml). The cells are incubated at 37° C. with shaking (240 rpm) until an OD₅₉₅ of 0.25 is obtained. Subsequently, IPTG is added to the culture to give a concentration of 1 mM and the cells are incubated for a further 4 hours. The cells are harvested by centrifugation (5000×g for 10 minutes). The cell pellet is taken up in 10 ml of PBS buffer containing 0.1 g of lysozyme (Sigma, Deisendorf) and 250 U of benzonase (Merck, Darmstadt) and the whole is incubated at 37° C. for 60 minutes.

FIG. 2 shows an SDS-PAGE carried out with mutS (arrow)-expressing E.coli strains after induction (lanes 1 and 2) and prior to induction (lane 3).

After that, 100 μl of PMSF (100 mM in isopropanol) (Sigma, Deisendorf) and 100 μl of Triton X-100 (Sigma, Deisendorf) were added. After the cells had been lysed, the cell remnants were centrifuged down at 10,000×g for 10 minutes. 2 ml of nickel-NTA-agarose (Qiagen, Hilden) are equilibrated 3× with 10 ml of buffer 17 (Qiagen, Hilden). The equilibrated nickel-NTA-agarose is subsequently added to the lysate. The whole is then incubated at 4° C. for one hour. The material containing bound mutS protein is separated off through a mini column having a glass frit (Biorad, Munich) and washed 3× with buffer A (4 ml, 2 ml and 2 ml). The protein is subsequently eluted with 2×2 ml of buffer B (Qiagen).

### Example: Labeling T.aquaticus mutS and E.coli mutS with Dye and Performing Functional Tests on them

a) Nonspecific Labeling of T.aquaticus mutS with Cy™3

Because of their fluorescence properties, the dyes Cy™3 and Cy™5 (Amersham Pharmacia Biotech, Little Chalfont, UK) are frequently used for the fluorescence labeling of biomolecules (Mujumdar, R. B. et al., Bioconjugate Chemistry 4 (1993) 105-111; Yu, H. et al., Nucleic Acids Research 22 (1994) 3226-3232). In this connection, the corresponding succinimidyl ester is usually linked, for the conjugation, nonspecifically and covalently, to protein lysine residues by means of a nucleophilic substitution reaction. For optimum fluorescence labeling of the protein in this context, the protocol worked out by Pharmacia (FluoroLink™ production specification protocol, Amersham Pharmacia Biotech, Little Chalfont, UK) envisages incubation of the protein with a large excess of fluorophore under conditions which are relatively strongly basic (0.1 M Na₂CO₃, pH 9.3). The thermostable Thermus aquaticus mutS was therefore first of all fluorescence-labeled with Cy™3 in accordance with this protocol. Following purification by gel permeation chromatography, and subsequent SDS-PAGE analysis, it was possible to detect a strongly fluorescent protein band which corresponded unambiguously, because of its molecular weight, to a mutS protein which was labeled with Cy™3 (FIG. 3). Lane 1 shows the mutS-Cy™3 (T.aquaticus), while lanes 2 to 4 show mutS-Cy™5 (E.coli).

However, the subsequent activity test (band shift assay) showed that the labeled protein no longer possessed any activity and was therefore not able to bind an oligomer which contained a G/T mispairing (see. FIG. 7). This experiment demonstrates that, in the case of labeling the mutS protein, the labeling procedure proposed by the dye manufacturer is not practicable and that a refined and optimized labeling protocol has to be established in order to preserve the active protein.

b) Nonspecific Labeling of E.coli mutS and T.aquaticus mutS with Cy™5

For the fluorescence labeling of the protein, four labeling assays using increasing concentrations of labeling reagent in labeling buffer (20 mM N-2-hydroxyethylpiperazine-N′-2-ethanesulfonic acid (HEPES), pH 7.9, 150 mM KCl, 10 mM MgCl₂, 0.1 mM N,N,N′,N′-ethylenediaminetetraacetate (EDTA), 10% glycerol in distilled water) were carried out in order to obtain different populations of fluorescence-labeled mutS. The activity of these proteins, which differed in their degree of labeling, was then investigated in the band shift assay.

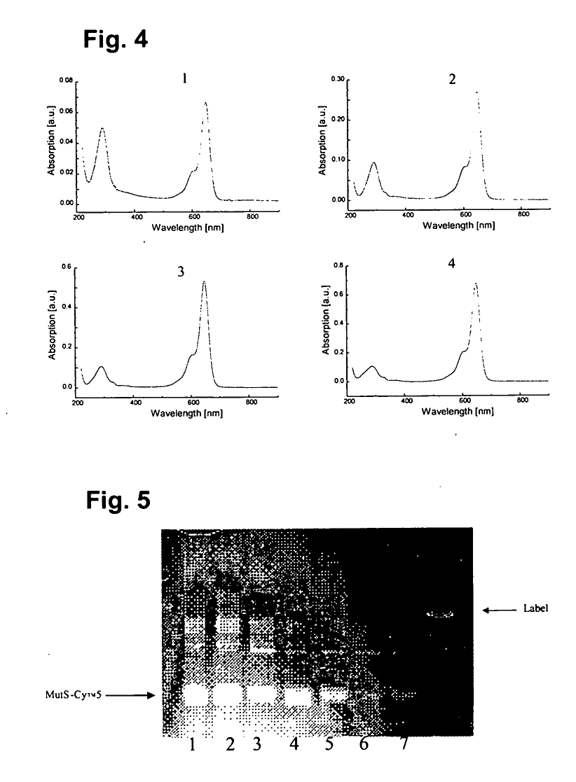

The labeling reaction was carried out, at room temperature for 30 minutes and in the dark, in a mixture (500 μl) consisting of E.coli mutS protein (50 μg, 1.05 μM) and increasing concentrations of Cy™5 succinimidyl ester (12 μM, 20 μM, 50 μM and 100 μM) in labeling buffer. For purifying the dye-labeled mutS protein, a NAP-5 gel filtration column (Pharmacia LKB Biotechnology, Uppsala, Sweden) was equilibrated with 3 column volumes of elution buffer (20 mM tris-HCl pH 7.6, 150 mM KCl, 10 mM MgCl₂, 0.1 mM EDTA, 1 mM dithiothreitol (DTT), 10% glycerol in distilled water). After elution buffer (500 μl) has been added to it, all the labeling reaction solution is loaded onto the column and the proteins, which were labeled to different extents with fluorescent dye, were isolated by eluting with elution buffer. The fluorescent protein fractions were then examined in more detail by UV spectrometry (FIG. 4) and SDS-PAGE gel chromatography (FIG. 5).

FIG. 4 shows examples of the UV spectra of different fractions of the mutS-Cy™5 (E.coli) conjugates, with different degrees of fluorescence labeling (D/P ratio), which were obtained in the labeling reactions. The spectra 1 to 4 show the following degrees of fluorescence labeling: 1. D/P=0.5 (20 μM Cy™5), 2. D/P=1.0 (50 μM Cy™5), 3: D/P=2.0 (100 μM Cy™5) and 4. D/P=3.0 (100 μM Cy™5).

FIG. 5 shows examples of SDS-PAGE carried out on different mutS-Cy™5 (E.coli) fractions having different degrees of fluorescence labeling (D/P ratio). Lanes 1 to 7 show the following degrees of fluorescence labeling: 1. D/P=0.5 (20 μM Cy™5), 2. D/P=0.5 (20 μM Cy™5), 3. D/P=1.5 (50 μM Cy™5), 4. D/P=1.0 (50 μM Cy™5), 5. D/P=2.0 (100 μm Cy™5), 6. D/P=3.0 (100 μM Cy™5), 7. D/P=2.5 (100 μM Cy™5).

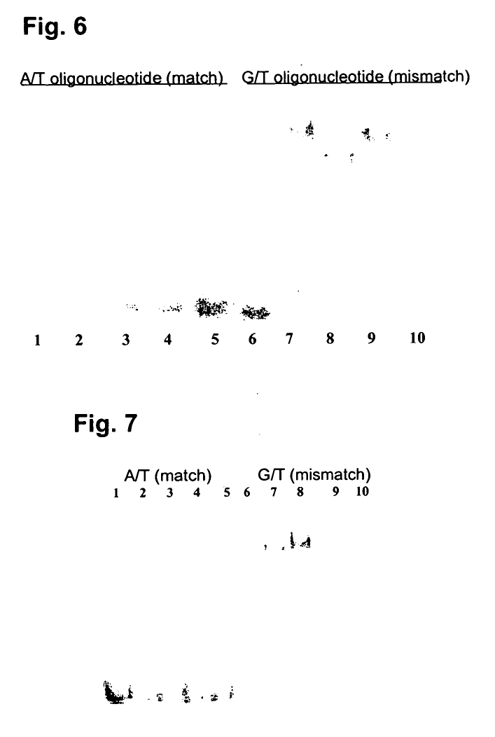

Subsequently, the band shift method was used to check whether the Cy™5-conjugated mutS proteins were functionally active, i.e. whether they were still able to bind specifically to base mispairings. For this, heteroduplexes were generated by hybridizing the oligonucleotides “OAT” (Seq. ID No. 6) and “GT” (Seq. ID No. 7), respectively, with the “sense” oligonucleotide (Seq. ID No. 11) by heating for 5 minutes at 95° C. in 10 M tris-HCl, 100 mM KCl, 5 mM MgCl₂, followed by cooling slowly down to room temperature. The “GT” oligonucleotide possesses a mutation as compared with the “AT” oligonucleotide. Using T₄ polynucleotide kinase (New England Biolabs, Frankfurt), 10 pmol each of the two heteroduplexes which wer produced using the “AT” and “GT” oligonucleotides were radioactively labeled, in accordance with the manufacturer's instructions, with 150 μCi of ³²P-ATP at their 5′ ends and purified through Sephadex G50 gel filtration columns (Pharmacia, Uppsala, Sweden) in accordance with the manufacturer's instructions. For a band shift assay, 17 fmol of the respective heteroduplex were taken up in 10 μl of reaction buffer (20 mM tris-HCl pH 7.6, 150 mM KCl, 10 mM MgCl₂, 0.1 mM EDTA, 1 mM dithiotreitol (DTT), 100 μg of BSA fraction VII/ml, 15% glycerol in distilled water) and this solution was incubated, at room temperature for 20 minutes, with 10 μl of the Cy™5-conjugated mutS proteins or with 10 μl, corresponding to 5.0 μg, of commercially obtainable mutS proteins. Unconjugated E.coli mutS (Gene Check, Fort Collins, USA) and T.aquaticus mutS (Epicentre, Madison, USA) were used, and Cy™5-conjugated T.aquaticus mutS was prepared, for comparison, using the protocol established for E.coli mutS. After the incubation, the mixtures were loaded onto 6% polyacrylamide gels and separated at 25 mA for 90 minutes. Gel and running buffer systems were 45 mM tris-borate, 10 mM MgCl₂, 1 mM EDTA. After the run, the gels were dried and analyzed by autoradiography (FIG. 6).

FIG. 6 shows Cy™5-conjugated E.coli mutS (lanes 4 and 9) which, as compared with commercially obtainable unlabeled protein (Gene Check, Fort Collins, USA, lanes 2 and 7) does not exhibit any loss of activity. The same applies to Cy™5-conjugated T.aquaticus mutS (Epicentre, Madison, USA, lanes 3 and 8 unconjugated, lanes 5 and 10 conjugated). Lanes 1 and 6 do not contain any protein. Both conjugated and unconjugated proteins bound markedly more strongly to the oligonucleotide containing the base mispairing (G/T) (lanes 6 to 10) than to the oligonucleotide without any mispairing (A/T) (lanes 1 to 5). Under the conjugation conditions employed here, neither E.coli mutS nor T.aquaticus mutS exhibited any loss of activity.

In contrast to the abovementioned conjugation protocol, the conjugation of T.aquaticus mutS with Cy™3 using the conditions which are recommended by the manufacturer of the dye, and which are suitable, for example, for antibodies, leads to the complete loss of the activity of the conjugated protein (FIG. 7), which means that it was not possible to use this standard protocol in the present case.

FIG. 7 shows unconjugated T.aquaticus mutS (lanes 2 and 7: 0.16 μg, lanes 5 and 10: 0.64 μg) and binds, in contrast to protein which is conjugated with Cy™3 under standard conditions (lanes 4 and 9: 0.16 μg, lanes 5 and 10: 0.64 μg), to DNA, with the DNA containing the base mispairing (lanes 6 to 10) being bound more effectively than the precisely pairing DNA (lanes 1 to 5). Lanes 1 and 6 do not contain any protein.

### Example: Alternative Method for Expressing Active mutS

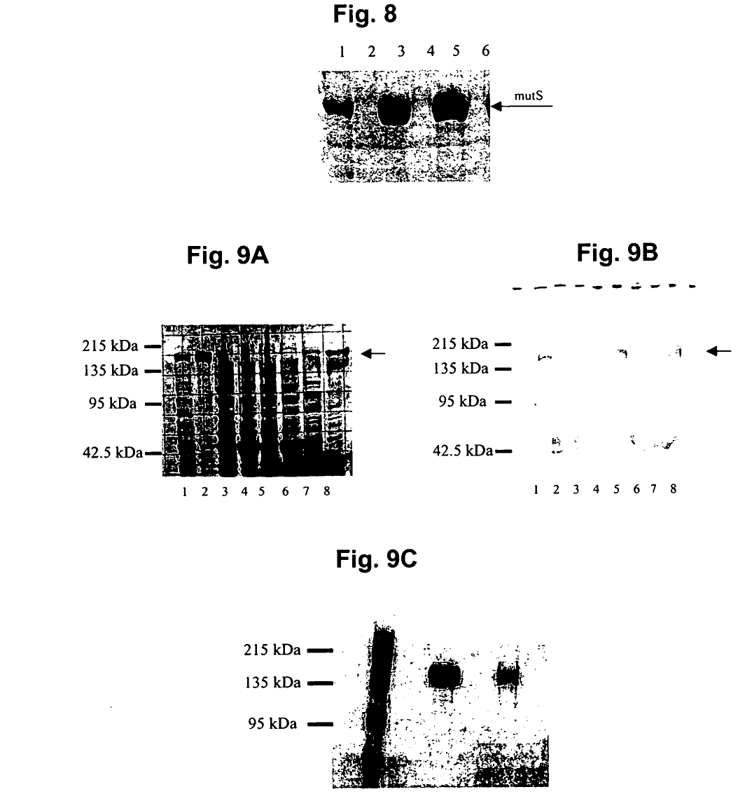

The overexpression of mutS in E. coli TOP10 led to the formation of insoluble protein. This problem, also termed inclusion body formation, occurs frequently in E. coli. FIG. 8 shows E. coli lysates which have been fractionated on SDS-PAGE and which were obtained from pQE30-mutS-transformed cultures which were grown at various temperatures and which overexpress mutS. The aim was to avoid the formation of inclusion bodies by using low incubation temperatures (30° C. and 25° C., respectively). However, if the soluble fraction of the lysates is considered, it can be seen that it only contains very small quantities of mutS protein. On the other hand, a very large quantity of mutS protein can be found in the insoluble fraction (inclusion bodies).

Since it is a very elaborate process to isolate soluble, and consequently functional, protein from inclusion bodies, it was necessary to find another expression system which generates more soluble protein.

FIG. 8 shows a Coomassie-stained 10% SDS-PAGE of E. coli lysates. Lane 1: insoluble fraction, lane 2 soluble fraction, from 25° C. cultures. Lane 3: insoluble fraction, lane 4 soluble fraction, from 30° C. cultures. Lane 5: insoluble fraction, lane 6 soluble fraction, from 37° C. cultures. All the pQE30-mutS transformed cultures were induced with 0.3 mM IPTG, and grown, for 3 h at the given temperatures.

For this reason, in a following step, a check was made to determine whether a change in the amino acid sequence of the expressed protein improves its solubility properties. First of all, it was tested whether a fusion protein consisting of the E. coli maftose-binding protein (MBP) and of E. coli mutS exhibited improved solubility properties. For this, the mutS-encoding DNA was inserted into the vector pMALc2x (NEB, Frankfurt, Seq. ID No. 8), resulting in the plasmid pMALc2x-mutS (Seq. ID. No. 9) Another advantage of this fusion protein as compared with the conventional mutS protein is the commercial availability of anti-MBP antibodies, which enable the fusion protein to be detected. An anti-mutS antibody is not at present obtainable commercially.

The fusion protein which was tested in this study consists of the 42 kDa maltose-binding protein (MBP) and the 92 kDa mutS protein.

For this, the DNA sequence which encodes E. coli mutS was amplified by PCR and isolated using standard methods. The 5′ BamHI primer (Seq. ID No. 52) introduces a BamHI cleavage site upstream of the start codon. At the same time, the nucleotide sequence located immediately upstream of the start codon is mutated such that the start codon function is lost. The purpose of this is to avoid the protein biosynthesis machinery initiating the formation of a truncated polypeptide at the start codon. The 3′ HindIII rev primer (Seq. ID No. 2) introduces a HindIII cleavage site downstream of the stop codon. The PCR is known to the skilled person and was carried out in accordance with the following scheme:

4 μl of E. coli genomic DNA (prepared in accordance with the manufacturer's instructions using the Qiagen genomic tip system 20/G from Qiagen, Hilden) are added to a 100 μl PCR mixture containing 61 μl of H₂O, 10 μl of 10 μM 5′ primer, 10 μl of 10 μM 3′ primer, 10 μl of 10×PCR buffer containing MgSO₄ (Roche, Mannheim), 2 μl of DMSO, 1 μl of dNTP's (25 mM in each case) and 2 μl of Pwo polymerase (=10 U). The PCR is run using the following program: 95° C. for 5 min followed by 30 cycles with 0.5 min, 95° C., 0.5 min, 55° C. and 2.5 min, 72° C. The end of the PCR is followed by an incubation of 7 min at 72° C. The mutS PCR product was subsequently isolated using a PCR purification kit (Qiagen, Hilden, Germany) and freed from salts, primers and proteins. The isolated DNA is quantified on a gel and cut with BamHI and HindIII:

for this, 41 μl of mutS PC R product (about 2 μg) is combined with 20 U of BamHI (2 μl, NEB, Frankfurt), 20 U of HindIII (2 μl, NEB, Frankfurt), and 5 μl 10×NEB2 buffer (NEB, Frankfurt) in a 50 μl mixture and the whole is incubated overnight at 37° C. In parallel with this, 10 μl of the vector pMALc2x (2 μg, from NEB, Frankfurt) are combined with 20 U of BamHI (2 μl, NEB, Frankfurt), 20 U of HindIII (2 μl, NEB, Frankfurt), 5 μl of 10×NEB2 buffer (NEB, Frankfurt) and 31 μl of water and the whole is incubated overnight at 37° C. The DNA fragments are subsequently purified on a 1% TBE agarose gel and freed from agarose residues using a QiaQuick gel extraction kit (Qiagen, Hilden). The DNA fragments were in each case taken up in 50 μl of water.

The quantities of pMALc2x and mutS are compared on an agarose gel and 200 ng of plasmid (3 μl) and 400 ng (14 μl) of mutS DNA are combined in a 20 μl ligation mixture containing 2 μl of 10× ligase buffer (Roche, Mannheim) and 1 μl of T4DNA ligase (2 U, Roche, Mannheim), and the whole is incubated at room temperature for 3 h. For the transformation, the E.coli k12 strain “Goldstar” (Stratagene, La Jolla, San Diego, USA) was shaken, and grown, overnight at 37° C. and 200 rpm in LB medium containing 100 μg of ampicillin/ml. On the following morning, 1 ml of the bacterial culture was transinoculated into 200 ml of fresh medium and shaken at 200 rpm, and at 37° C., until an optical density of 0.565 was obtained at 595 nm. The culture was subsequently cooled down to 4° C. and centrifuged down at 2500×g. The supematant was discarded and the pelleted bacteria were taken up in 7.5 ml of LB medium containing 10% (w/v) polyethylene glycol 6000, 5% dimethyl sulfoxide, 10 mM MgSO₄, 10 mM MgCl₂ (Promega, Madison, USA), pH 6.8 with this suspension then being incubated on ice for one hour, then shock-frozen in liquid nitrogen and stored at −80° C. For the transformation, 10 μl of the ligation mixture were taken up in 100 μl of 100 mM KCl, 30 mM CaCl₂, 50 mM MgCl₂ and incubated with 100 μl of the thawed bacteria on ice for 20 min. After a 10 minute incubation at room temperature, 1 ml of LB medium was added to the bacteria and the latter were then incubated at 37° C. for one hour while being shaken. Subsequently, the mixture was streaked out on LB agar plates containing 100 μg of ampicillin/ml and these plates were incubated overnight at 37° C. Individual colonies were isolated and propagated, overnight at 37° C., in 3 ml of LB medium containing 100 μg of ampicillin/ml. The plasmid DNA was isolated from the bacteria, and purified, using the QIAprep Spin Miniprep kit (Qiagen/Hilden) in accordance with the manufacture's instructions. The plasmid content of positive clones is investigated by means of miniprep analysis. Protein induction was performed on four independently isolated clones which harbored the desired pMALc2x-mutS plasmid (Seq. ID. No. 9). A 5 ml LB (containing 100 μg of ampicillin/ml) overnight culture of E. coli Goldstar harboring the plasmid pMALc2x-mutS is diluted 1:50 and grown to an OD₅₉₅=0.5 at 37° C. while shaking (240 rpm). Subsequently, Lämmli sample buffer is added to an aliquot of each culture; IPTG is then added to the cultures to give a concentration of 0.3 mM and the cells are incubated at 37° C. for a further 2 h. Lämmli sample buffer is subsequently added to the cultures and the latter are fractionated on SDS-PAGE together with the uninduced sample. Coomassie staining of the gels demonstrates the expression of an approximately 140 kDa (42 kDa MBP+93 kDa mutS) protein in clones 1 and 6 (FIG. 9A). An SDS gel which was loaded with identical samples, and which was fractionated in parallel, was analyzed by Western blotting using a first anti-MBP antibody (reagents and methodology described in: pMAL Protein Fusion and Purification System Handbook, NEB, Frankfurt). In this connection, it was possible to detect a protein of about 140 kD in size in the case of clones 1 and 6 (FIG. 9B), which protein is consequently the MBP/mutS fusion protein. However, initial experiments showed that, in this expression system as well, the majority of the expressed mutS fusion protein was present in the insoluble inclusion bodies, something which was possibly due to the cells which were employed in this case (data not shown). For this reason, the plasmid pMALc2x-mutS was isolated from clone 2 using the Qiagen Midi-Prep Kit (Qiagen/Hilden) and transformed, as described above, into competent E.coli C600 cells. This strain has less tendency to form inclusion bodies. A freshly transformed clone was grown overnight, at 37° C., in 100 ml of LB medium containing 0.2% glucose, 2mM MgCl₂, and 100 μg of ampicillin/ml. 50 ml of this culture were harvested by centrifugation and grown, at 37° C., in 3 L of this medium up to an OD OD₅₉₅=0.6. After having added IPTG to a concentration of 0.3 mM, the cells were subsequently incubated at 30° C. for 3 h while being shaken, then harvested by centrifugation and resuspended in 100 ml of column buffer (20 mM HEPES pH 7.9, 150 mM KCl, 10 mM MgCl₂, 0.1 mM EDTA, 1 mM dithiothreitol, 0.2 mM PMSF). After that, the cells were lysed by ultrasonication and cell debris were separated off by centrifuging at 9000×g.

The MBP-mutS fusion protein was subsequently purified by affinity chromatography on an amylose column and eluted in column buffer (see above) containing 10 mM maltose (described in: pMAL Protein Fusion and Purification System Handbook, NEB, Frankfurt). After that, the Bradford Assay Kit (Biorad, Munich) was used to determine the protein concentration in the eluat 0.2 μg of the eluate were analyzed by SDS-PAGE. In this connection, it was found that the fusion protein contains only few contaminating proteins (FIG. 9C). The MBP-mutS fusion protein was treated 1:1 (v/v) with glycerol and stored at −20° C. The activity of the proteins was verified using the “band-shift” method and also surface plasmon resonance technology (see below).

FIG. 9A shows a Coomassie-stained 5%-20% SDS-PAGE of E. coli lysates derived from pMALc2x-mutS-transformed cells. Lanes 1 and 2: clone 1. Lanes 3 and 4: clone 4. Lanes 5 and 6: clone 5. Lanes 7 and 8: clone 6. Prior to the lysis, the cells were either not induced (lanes 1, 3, 5 and 7) or induced with 0.3 mM IPTG (lanes 2, 4, 6 and 8). A protein of the expected size of 140 kDa is formed in clones 1 and 6 (arrow). FIG. 9B: Western blot analysis of a 5%-20% SDS-PAGE of E. coli lysates derived from pMALc2x-mutS-transformed cells. Lanes 1 and 5: clone 1. Lanes 2 and 6: clone 4. Lanes 3 and 7: clone 5. Lanes 4 and 8: clone 6. Prior to the lysis, the cells were either not induced (lanes 1-4) or induced with 0.3 mM IPTG (lanes 5-8). A protein of the expected size of 140 kDa was recognized by the anti-MBP antibody (arrow) in the case of clones 1 and 6. A protein of the size of MBP (about 40 kDa) is recognized in the case of clones 4 and 5. FIG. 9C: Coomassie-stained 5%-20% SDS-PAGE of purified MBP-mutS fusion proteins. Affinity chromatography-purified MBP-mutS from 2 independent preparations was investigated by gel electrophoresis. Lane 1: marker. Lane 2: preparation 1. Lane 3: preparation 2.

### Examples: Other Labeling Methods

2 mg of mutS protein (either an MBP-fused protein or an unfused variant, from Genescan (Fort Collins, USA), commercially acquired E.coli protein, or T. aquaticus mutS obtained from Biozym, Hess, Oldendorf) were dissolved in 18 ml of 20 mM HEPES pH 7.9, 5 mM MgCl₂, 150 mM KCl, 10% (v/v) glycerol. 250 nmol of Cy™5-succinimidyl ester were dissolved in 2 ml of the same buffer, with this solution then being mixed thoroughly with the solution of the protein and the whole being incubated at room temperature for 30 minutes. Subsequently, 2 ml of 20 mM HEPES pH 7.9, 5 mM MgCl₂, 150 mM KCl, 100 mM adenosine triphosphate, 10 mM dithiothreitol, 10% (v/v) glycerol was added to the reactions. The protein-containing solutions were dialyzed, at 4° C., 2× for 3 hours and also 1× overnight against in each case 21 of 20 mM tris pH 7.6, 5 mM MgCl₂, 150 mM KCl, 1 mM DTT, 10% (v/v) glycerol in a dialysis bag having a cut-off of 10 kDa. After that, an equal volume of glycerol was added to the protein and the whole was stored at −20° C.

### Example: Detecting Point Mutations on Electronically Addressable DNA Chips

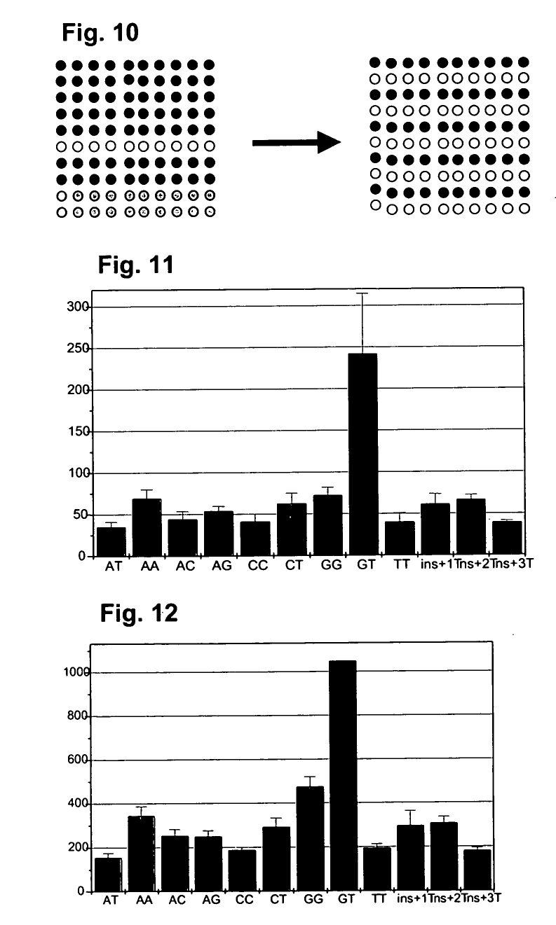

The functional dye-labeled E. coli and T. aquaticus mutS proteins were now used for detecting point mutations on electronically addressable DNA chips. For this, a 100 nM solution of the “sense” oligonucleotide (Seq. ID No. 10), which had been biotinylated at the 5′ end, was first of all electronically addressed, for 60 s using 2 V, to all the positions in rows 1-5 and 7-10 of a 100-position DNA chip supplied by Nanogen (as described in Radtkey et al., Nucl. Acids Res. 28, 2000, e17; R. G. Sonowsky et al., Proc. Natl. Acad. Sci. USA, 1119-1123; 1994 P. N. Gilles et al., Nature Biotechnol. 17, 365-370, 1999) (a diagram in this regard is given in FIG. 10, which shows the use of electronic addressing to load the 100-position chip with DNA). The current strength per position varied between 262 nA and 364 nA in rows 1-8 and between 21 nA and 27 nA in rows 9 and 10, resulting in less DNA being addressed to these positions (FIG. 10, left-hand matrix, the two lower rows). Subsequently, the oligonucleotide Seq. ID No. 6 which was completely complementary to the “sense” oligonucleotide” (Seq. ID No. 10), and was labeled with Cy™3 at the 5′ end, was applied to rows 1, 3, 7 and 9 of the chip under the above-described conditions such that completely paired double strands, designated “AT”, were formed at these positions (Table 1 and FIG. 10, right-hand matrix, darkly shaded). In a further step, the Cy™3-labeled oligonucleotide Seq. ID No. 7 was applied, as described above, to rows 2, 4, 8 and 10 of the chip. With the previously addressed “sense” oligonucleotide, this oligonucleotide forms a double strand having a single G/T base mispairing, for which reason the resulting double-stranded oligonucleotide is termed “GT” (Table 1, FIG. 10, right-hand matrix, shaded lightly). Row 5 (Table 1, FIG. 10) consequently contains single-stranded “sense” DNA while row 6 (Table 1, FIG. 10) does not contain any DNA. 50 μl of each of the above-described Cy™5-labeled mutS proteins were purified on Sephadex G50 spin columns (Pharmacia, Uppsala, Sweden) in accordance with the manufacturer's instructions and consequently completely freed from unconjugated dye. The columns were equilibrated beforehand with 500 μl of buffer (20 mM tris-HCl pH 7.6, 150 mM KCl, 10 mM MgCl₂ 0.1 mM EDTA, 1 mM dithiothreitol (DTT)), while the purified protein was added to the chip surface and incubated at room temperature for 20 minutes. Subsequently, the intensity of the Cy™3 fluorescence was measured on the chip surface in accordance with the manufacturer's (i.e. Nanogen's) instructions (Table 1). The small variations in the values in rows 1 to 4 and 7 and 8 point to uniform hybridization of the double-stranded AT and GT oligonucleotides. The comparatively lower values in rows 9 and 10 reflect the smaller quantities of DNA in these rows as a result of the lower addressing current strength (see above). The lower values in row 5 constitute the background signal, since no fluorescence-labeled DNA was addressed to this row. As the control, row 6 only contains single-stranded “sense” DNA.

TABLE 1
Determining the fluorescence intensity of Cy ™ 3-labeled DNA on an
electronically addressed 100-position chip at 575 nm. The table shows the
positions on the chip together with the appurtenant relative fluorescent
intensities, and also the DNA addressed to these positions (outer right-hand column).

| Pos. | 1 | 2 | 3 | 4 | 5 | 6 | 7 | 8 | 9 | 10 | DNA |
|---|---|---|---|---|---|---|---|---|---|---|---|
| 1 | 913.16 | 879.46 | 808.750 | 975.325 | 937.325 | 920.182 | 932.976 | 982.548 | 920.258 | 914.798 | AT |
| 2 | 900.79 | 929.97 | 957.315 | 913.726 | 909.292 | 909.471 | 908.465 | 904.025 | 902.127 | 900.796 | GT |
| 3 | 900.79 | 902.93 | 909.391 | 912.882 | 917.427 | 910.059 | 911.066 | 905.345 | 901.695 | 900.797 | AT |
| 4 | 900.79 | 904.34 | 908.760 | 914.050 | 914.643 | 909.385 | 909.835 | 907.314 | 900.797 | 900.796 | GT |
| 5 | 24.537 | 26.288 | 26.833 | 28.884 | 29.762 | 29.525 | 29.281 | 27.904 | 29.039 | 28.188 | only sense |
| 6 | 5.407 | 5.110 | 5.418 | 5.365 | 5.382 | 5.377 | 5.657 | 5.445 | 5.521 | 5.433 | none |
| 7 | 900.79 | 902.33 | 915.767 | 912.943 | 908.588 | 910.843 | 908.263 | 903.791 | 900.913 | 900.642 | AT |
| 8 | 900.79 | 900.81 | 904.148 | 909.292 | 919.081 | 908.596 | 909.668 | 904.243 | 900.796 | 900.797 | GT |
| 9 | 37.419 | 33.100 | 26.864 | 25.072 | 24.536 | 25.269 | 25.699 | 28.185 | 26.905 | 31.072 | AT |
| 10 | 36.782 | 32.574 | 28.463 | 30.519 | 32.815 | 38.168 | 32.349 | 29.213 | 27.744 | 33.626 | GT |

The subsequent measurement of the fluorescence of the Cy™5 -labeled E. coli mutS protein shows clearly that the protein preferably binds to the GT oligonucleotide (Table 2). Even when the quantities of DNA are very small (Table 2, rows 9 and 10), it is possible to use the dye-labeled protein to discriminate between the perfectly pairing oligonucleotide (AT) and the oligonucleotide (GT) which generates a base mispairing, with this discrimination becoming even clearer when the DNA quantities are larger (rows 1-4 and 7 and 8, compare Table 1). The low background values are also striking (Table 2, rows 5 and 6). In order to ascertain whether other base mispairing-binding proteins, such as the T. aquaticus mutS protein, can also be used for rapidly detecting mutations on electronically addressable chips, a 100-position chip from Nanogen was first of all addressed precisely as described above. Subsequently, Cy™5-labeled T. aquaticus mutS protein was further purified on Sephadex G50 spin columns, as described above, and then incubated with the chip surface for 20 min. Subsequently, the intensity of the Cy™3 fluorescence on the chip surface was measured in accordance with the manufacturer's, i.e. Nanogen's, instructions (Table 3). The small variations in the values in rows 1-4 and 7 and 8 once again point to uniform hybridization of the double-stranded AT and GT oligonucleotides. The comparatively lower values in rows 9 and 10 once again reflect the fact that the quantities of DNA in these rows are lower because of the lower addressing current strength employed (see above). The low values in rows 5 and 6 constitute the background signal since no fluorescence-labeled DNA was addressed to these rows. Subsequent measurement of the fluorescence of the Cy™5-labeled T. aquaticus mutS protein shows clearly that this protein also binds preferentially to the GT oligonucleotide (Table 4) and is consequently suitable for detecting mutations on electronically addressable DNA chips.

TABLE 2
Determining the intensity of the fluorescence of Cy ™ 5-labeled
E. coli mutS-protein on an electronically addressed 100-position chip
at 670 nm. The table shows the positions on the chip together with the
appurtenant relative fluorescence intensities, and also the DNA which is
addressed to these positions (outer right-hand column).

| Position | 1 | 2 | 3 | 4 | 5 | 6 | 7 | 8 | 9 | 10 | DNA |
|---|---|---|---|---|---|---|---|---|---|---|---|
| 1 | 5.007 | 7.536 | 10.330 | 10.316 | 11.143 | 11.207 | 10.945 | 11.963 | 10.038 | 9.631 | AT |
| 2 | 20.460 | 21.050 | 20.954 | 18.520 | 22.295 | 21.577 | 19.593 | 20.539 | 21.096 | 26.274 | GT |
| 3 | 10.936 | 9.856 | 9.906 | 9.983 | 10.302 | 10.393 | 10.680 | 10.542 | 10.750 | 10.379 | AT |
| 4 | 22.551 | 17.807 | 15.700 | 17.310 | 20.853 | 18.344 | 18.827 | 20.660 | 20.503 | 22.828 | GT |
| 5 | 6.716 | 7.086 | 7.244 | 7.580 | 7.269 | 6.859 | 7.318 | 7.563 | 6.927 | 6.899 | only |
| | | | | | | | | | | | sense |
| 6 | 5.351 | 5.029 | 5.792 | 5.446 | 5.442 | 5.465 | 6.156 | 5.581 | 5.347 | 5.446 | none |
| 7 | 11.076 | 9.752 | 10.898 | 10.236 | 10.419 | 10.523 | 10.880 | 10.792 | 11.788 | 11.811 | AT |
| 8 | 21.663 | 20.410 | 19.348 | 19.426 | 18.322 | 17.861 | 20.328 | 19.522 | 21.067 | 27.832 | GT |
| 9 | 8.960 | 8.881 | 8.308 | 8.256 | 8.733 | 8.808 | 8.912 | 8.249 | 8.860 | 8.978 | AT |
| 10 | 16.046 | 12.316 | 13.123 | 15.887 | 13.223 | 12.747 | 13.535 | 12.519 | 14.172 | 14.429 | GT |

TABLE 3
Determining the intensity of the fluorescence of Cy ™ 3-labeled DNA on an electronically
addressed 100-position chip at 575 nm. The table shows the positions on the chip
together with the appurtenant relative fluorescence intensities and also the DNA which
is addressed to these positions (outer right-hand column).

| Position | 1 | 2 | 3 | 4 | 5 | 6 | 7 | 8 | 9 | 10 | DNA |
|---|---|---|---|---|---|---|---|---|---|---|---|
| 1 | 942.22 | 939.12 | 924.103 | 909.110 | 911.722 | 909.437 | 906.431 | 975.994 | 900.796 | 900.796 | AT |
| 2 | 900.79 | 902.609 | 913.694 | 910.908 | 912.856 | 908.688 | 911.264 | 905.132 | 913.865 | 900.796 | GT |
| 3 | 900.79 | 900.79 | 903.957 | 908.821 | 917.991 | 914.377 | 909.321 | 906.641 | 900.796 | 900.797 | AT |
| 4 | 900.79 | 905.52 | 904.156 | 909.869 | 916.580 | 911.754 | 912.818 | 926.430 | 901.621 | 900.797 | GT |
| 5 | 26.150 | 28.571 | 30.898 | 32.025 | 30.008 | 29.247 | 29.646 | 27.469 | 21.867 | 20.293 | only |
| | | | | | | | | | | | sense |
| 6 | 8.180 | 9.125 | 8.162 | 8.388 | 7.798 | 7.268 | 7.022 | 6.862 | 6.891 | 6.900 | none |
| 7 | 900.64 | 900.79 | 903.481 | 905.261 | 906.195 | 903.969 | 904.785 | 901.280 | 900.796 | 900.797 | AT |
| 8 | 900.79 | 981.94 | 900.895 | 905.638 | 907.985 | 904.855 | 904.237 | 900.958 | 900.797 | 900.796 | GT |
| 9 | 42.681 | 35.576 | 29.910 | 27.724 | 30.742 | 31.228 | 29.570 | 31.958 | 34.295 | 41.122 | AT |
| 10 | 45.187 | 34.862 | 34.221 | 35.343 | 36.580 | 34.221 | 37.473 | 42.224 | 38.967 | 45.342 | GT |

TABLE 4
Determining the intensity of the fluorescence of Cy ™ 5-labeled T. aquaticus mutS
protein on an electronically addressed 100-position chip at 670 nm. The table shows
the positions on the chip together with the appurtenant relative fluorescence intensities
and also the DNA which is addressed to these positions (outer right-hand column).

| Position | 1 | 2 | 3 | 4 | 5 | 6 | 7 | 8 | 9 | 10 | DNA |
|---|---|---|---|---|---|---|---|---|---|---|---|
| 1 | 18.674 | 12.970 | 19.794 | 14.694 | 19.651 | 19.054 | 15.542 | 16.099 | 14.511 | 14.478 | AT |
| 2 | 30.716 | 25.352 | 31.469 | 23.856 | 24.055 | 23.581 | 30.308 | 27.575 | 30.865 | 30.406 | GT |
| 3 | 17.934 | 14.574 | 147.07 | 23.270 | 13.705 | 13.979 | 14.102 | 13.887 | 14.227 | 22.232 | ATT |
| 4 | 37.450 | 25.603 | 23.112 | 30.057 | 22.500 | 21.659 | 23.151 | 211.889 | 24.264 | 28.019 | GT |
| 5 | 16.433 | 12.215 | 11.160 | 10.775 | 10.807 | 10.457 | 10.633 | 19.564 | 66.942 | 9.961 | only sense |
| 6 | 14.202 | 15.956 | 9.740 | 10.033 | 9.942 | 8.871 | 8.562 | 8.584 | 9.151 | 7.928 | none |
| 7 | 15.351 | 155.31 | 15.697 | 15.485 | 14.713 | 15.312 | 14.260 | 14.290 | 15.319 | 14.714 | AT |
| 8 | 34.002 | 36.044 | 48.178 | 29.189 | 27.061 | 31.851 | 30.973 | 32.677 | 33.289 | 49.391 | GT |
| 9 | 11.923 | 16.883 | 13.129 | 12.510 | 13.621 | 13.467 | 14.091 | 14.914 | 12.646 | 11.692 | AT |
| 10 | 22.538 | 169.95 | 22.396 | 19.452 | 25.154 | 26.653 | 25.365 | 27.410 | 26.602 | 23.361 | GT |

### Example: Detecting Mutations in DNA Molecules on Electronically Addressable Chips in Dependence on the Salt Conditions

Comparison of the binding of E. coli mutS to base mispairings under high-salt conditions and under low-salt conditions:

In order to improve measurement accuracy, the binding conditions of the dye-labeled mutS protein were investigated and optimized so as to ensure that base mispairings on the surface of an electronically addressable chip are recognized even more efficiently.

For this, two electronically addressable chips, which were supplied by Nanogen and whose surfaces consisted of an agarose layer in which streptavidin molecules were embedded, were first of all loaded with the biotinylated “sense” oligonucleotide (Seq. ID No. 10) and then hybridized with the Cy™3-labeled “AT” (Seq. ID No. 12, counterstrand for perfect basepairing) or GT (Seq. ID No. 13, counterstrand for GT base mispairing) oligonucleotides. Subsequently, the binding of mutS to the resulting DNA double strands was tested at two different salt concentrations. For this, the oligonucleotides are first of all dissolved, at a concentration of 100 nM, in histidine buffer and this solution is incubated at 95° C. for 5 min. The “sense” oligonucleotide was electronically addressed to defined positions on both agarose chips, for 60 sec and at a voltage of 2.0 V, in the Nanogen workstation loading appliance; the hybridization with the “AT” or “GT” second-strand oligonucleotides was performed in the same way but at 2.0 V for 120 sec. The loading scheme, which was identical for both chips, is shown in Table 5. After having been loaded, the chips were taken out of the loading appliance and in each case filled with 1 ml of phosphate blocking buffer and incubated at room temperature for 45 min in order to saturated nonspecific protein-binding sites.

One of the chips (termed chip A below) was subsequently washed with 0.5 ml of high-salt buffer and incubated, at 37° C. for 20 min, with a mixture consisting of 20 μl Cy™5-labeled E.coli MBP-mutS (concentration: 0.45 μg/μl)+79 μl of high-salt buffer+1 μl of 100×BSA (New England Biolabs, Frankfurt am Main). After that, the chip was washed by hand, at room temperature, with 135 ml of high-salt buffer. The second chip (chip B) was treated in accordance with the same protocol but using low-salt buffer instead of the high-salt buffer.

Finally, the Cy™5 fluorescence intensities on the surfaces of the two chips were measured in the Nanogen reader. The following appliance settings were used for the measurement: high sensitivity (“high gain”); 256 μs integration time. The measured values are given in Table 6 (for chip A) and in Table 7 (for chip B), with the statistical analysis of the results being shown in Table 8.

Histidine buffer: 50 mM L-histidine (Sigma, Deisenhofen); this solution was filtered through a membrane having a pore size of 0.2 μm and degassed under negative pressure

Phosphate blocking buffer: 50 mM NaPO₄, pH 7.4/500 mM NaCl/3% Bovine serum albumen (BSA; from Serva, Heidelberg)

Low-salt buffer: 20 mM tris, pH 7.6/50 mM KCl/5 mM MgCl₂/0.01% Tween-20/1 mM DTT

High-salt buffer: 20 mM tris, pH 7.6/300 mM KCl/5 mM MgCl₂/0.01% Tween-20/1 mM DTT

TABLE 5
Scheme for loading chips A and B: “AT”: perfect pairing. Positions
were addressed with sense and subsequently hybridized with
“AT”: GT: GT-mispairing. Positions were addressed with sense
and then hybridized with GT: sense: single-stranded sense.
Positions were addressed with sense but not hybridized with
any counterstrands; SS “AT”: single-stranded “AT”.
Positions were only loaded with “AT”: without there being
any biotinylated first strand.

| Position | 1 | 2 | 3 | 4 | 5 | 6 | 7 | 8 | 9 | 10 |
|---|---|---|---|---|---|---|---|---|---|---|
| 1 | GT | AT | AT | GT | GT | AT | AT | GT | GT | AT |
| 2 | AT | GT | GT | AT | AT | GT | GT | AT | AT | GT |
| 3 | GT | AT | AT | GT | GT | AT | AT | GT | GT | AT |
| 4 | AT | GT | GT | AT | AT | GT | GT | AT | AT | GT |
| 5 | sense | sense | sense | sense | sense | sense | sense | sense | sense | sense |
| 6 | ssAT | ssAT | ssAT | ssAT | ssAT | ssAT | ssAT | ssAT | ssAT | ssAT |
| 7 | GT | AT | AT | GT | GT | AT | AT | GT | GT | AT |
| 8 | AT | GT | GT | AT | AT | GT | GT | AT | AT | GT |
| 9 | sense | AT | AT | sense | sense | AT | AT | sense | sense | AT |
| 10 | AT | sense | sense | AT | AT | sense | sense | AT | AT | sense |

TABLE 6
Measuring the red fluorescence intensity of chip A for detecting the binding
of Cy ™ 5-labeled E. coli MBP-mutS to perfectly paired or
GT-mispaired DNA double strands under high-salt conditions. The table shows the
positions on the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 44.007 | 56.342 | 69.989 | 85.363 | 87.951 | 78.753 | 75.654 | 87.145 | 85.769 | 54.169 |
| 2 | 42.705 | 60.958 | 81.111 | 84.320 | 81.805 | 83.464 | 81.613 | 71.096 | 57.087 | 62.442 |
| 3 | 51.512 | 61.777 | 72.146 | 84.844 | 76.928 | 76.482 | 61.824 | 74.533 | 77.773 | 55.655 |
| 4 | 49.892 | 69.783 | 71.486 | 69.254 | 61.875 | 67.159 | 66.752 | 61.915 | 62.737 | 65.770 |
| 5 | 49.067 | 58.874 | 64.822 | 56.678 | 51.375 | 46.336 | 47.167 | 48.650 | 53.223 | 46.837 |
| 6 | 107.484 | 118.344 | 123.665 | 119.388 | 107.549 | 98.146 | 97.272 | 99.218 | 96.022 | 93.144 |
| 7 | 73.030 | 83.649 | 71.361 | 74.500 | 68.791 | 58.536 | 54.627 | 60.832 | 60.482 | 52.118 |
| 8 | 72.017 | 90.977 | 78.122 | 66.356 | 65.933 | 66.362 | 65.426 | 56.053 | 52.914 | 53.851 |
| 9 | 50.033 | 69.534 | 74.262 | 60.324 | 55.581 | 62.312 | 56.824 | 45.303 | 45.792 | 52.180 |
| 10 | 56.835 | 58.295 | 67.034 | 66.505 | 66.981 | 51.965 | 49.338 | 49.710 | 49.502 | 34.009 |

TABLE 7
Measuring the red fluorescence intensity of chip B for detecting the binding of
Cy ™ 5-labeled E. coli-MBP-mutS to perfectly paired or
GT-mispaired DNA double strands under low-salt conditions. The table shows
the positions on the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 183.805 | 84.682 | 95.623 | 247.683 | 238.920 | 80.772 | 83.528 | 213.899 | 163.858 | 57.516 |
| 2 | 76.116 | 225.902 | 231.895 | 90.443 | 87.444 | 243.584 | 238.834 | 83.615 | 68.113 | 128.046 |
| 3 | 187.665 | 94.496 | 99.090 | 232.073 | 237.153 | 81.999 | 86.631 | 236.370 | 222.093 | 68.505 |
| 4 | 87.734 | 219.456 | 207.951 | 88.916 | 81.739 | 236.754 | 237.001 | 96.251 | 91.209 | 198.486 |
| 5 | 52.912 | 62.258 | 59.499 | 56.295 | 54.568 | 51.441 | 51.618 | 51.713 | 54.205 | 49.923 |
| 6 | 121.241 | 111.344 | 132.387 | 121.410 | 108.099 | 103.885 | 110.848 | 109.597 | 113.118 | 112.430 |
| 7 | 188.129 | 91.299 | 89.143 | 246.113 | 243.621 | 79.033 | 78.357 | 243.554 | 228.903 | 73.129 |
| 8 | 82.949 | 243.387 | 252.841 | 92.315 | 96.304 | 228.550 | 231.000 | 92.587 | 86.673 | 201.766 |
| 9 | 55.515 | 84.376 | 86.868 | 55.983 | 50.664 | 76.616 | 70.750 | 50.229 | 50.197 | 66.151 |
| 10 | 72.759 | 61.065 | 58.550 | 74.392 | 78.886 | 47.839 | 45.217 | 61.067 | 73.787 | 40.497 |

TABLE 8
Statistical analysis of the results obtained with chip A and chip B.
The mean values and standard deviations of the fluorescence intensities
at all the positions with the same loading were calculated in each case.

| | Chip A-high-salt | Chip B-low-salt |
|---|---|---|
| Perfect pairing | 63.6 +/− 10.1 | 82.3 +/− 9.9 |
| GT-mispairing | 71.9 +/− 11.7 | 221.3 +/− 27.7 |
| Sense single strand | 52.0 +/− 7.4 | 53.0 +/− 5.2 |
| AT single strand | 106.0 +/− 10.5 | 114.4 +/− 7.9 |

It is evident from Table 8 that, under low-salt conditions (50 mM KCl), E.coli MBP-mutS binds more strongly to GT-mispaired DNA double strands than it does to perfectly paired double strands or to single-stranded DNA. On the other hand, it was only possible to demonstrate a slight preferential binding of the mutS protein to mispaired DNA double strands at the higher salt concentration (300 mM KCl). This is surprising insofar as relatively high salt concentrations are usually employed in the literature for binding mutS. However, in the case of the chips which are used in the present instance, there is presumably a nonspecific hydrophobic interaction between the protein and the agarose permeation layer of the chip, with this nonspecific interaction preventing good penetration of the chip under high salt conditions. For this reason, buffers containing low salt concentrations were used for all the following experiments.

### Example: The use of mutS to Recognize Different Base Mispairings

This experiment demonstrates that mutS protein can also be employed for detecting other base mispairings or insertions/deletions on DNA chips. For this, the following types of DNA double strands were produced by hybridization at the different positions on an electronically addressable agarose chip supplied by Nanogen:

completely complementary double strands

double strands which contain one of the eight possible base mispairings (AA, AG, CA, CC, CT, GG, GT, TT)

double strands in which one strand contains an insertion of 1, 2 or 3 bases.

The ability of E.coli mutS to bind to the resulting DNA double strands was then tested. For this, the first and second strand oligonucleotides were firstly dissolved, at a concentration of 100 nM, in histidine buffer and denatured at 95° C. for 5 min. The biotinylated “sense” oligonucleotide (Seq. ID No. 10) was electronically addressed to the individual positions on the agarose chip, for 60 sec and at a voltage of 2.0 V, in the loading appliance of the Nanogen workstation. The hybridization with the “AF” (Seq. ID No. 12), GT (Seq. ID No. 13), AA (Seq. ID No. 14), AG (Seq. ID No. 15), CA (Seq. ID No. 16), CC (Seq. ID No. 17), CT (Seq. ID No. 18), GG (Seq. ID No. 19), TT (Seq. ID No. 20), ins+1T (Seq. ID No. 21), ins+2T (Seq. ID No. 22) and ins+3T (Seq. ID No. 23) second strand nucleotides was carried out at 2.0 V for 120 sec. The loading scheme is shown in Table 9; The name of each second-strand oligonucleotide indicates the mispairing or insertion (“ins”) which arises during the hybridization.

After the loading, the chip was taken out of the loading appliance, filled with 1 ml of blocking buffer and incubated at room temperature for 60 min in order to saturate nonspecific protein-binding sites. Subsequently, the chip was incubated, at room temperature for 60 min, with 10 μl of Cy™5-labeled E.coli mutS (concentration: 50 ng/μl) in 90 μl of incubation buffer. After this incubation, the chip was washed by hand with 1 ml of incubation buffer and then inserted into the Nanogen reader and washed, at a temperature of 37° C. 70× with in each case 0.5 ml of washing buffer. Finally, the Cy™5 fluorescence intensities at the individual positions on the chip were measured in the Nanogen reader using the following appliance settings: high sensitivity (“high gain”): 256 μs integration time. The relative fluorescence intensities which were measured are given in Table 10; the results of the statistical analysis of the measurement data are shown in Table 11 and in FIG. 11.

Histidine buffer: 50 mM L-histidine; this solution was filtered through a membrane having a pore size of 0.2 μm and degassed by negative pressure.

Blocking buffer: 20 mM tris, pH 7.6/50 mM KCl/5 mM MgCl₂/0.01% Tween-20/3% BSA (Serva, Heidelberg)

Incubation buffer: 20 mM tris, pH 7.6/50 mM KCl/5 mM MgCl₂/0.01 % Tween-20/1% BSA

Washing buffer: 20 mM tris, pH 7.6/50 mM KCl/5 mM MgCl₂/0.1% Tween-20

TABLE 9
Scheme for loading a chip for detecting the binding of Cy ™ 5-labeled
E. coli mutS to different base mispairings. “Neg”: positions which are
not loaded with DNA. “ssDNA”: positions which are only loaded with
the “sense” single strand. All the remaining positions were first of all
addressed with the “sense” oligonucleotide and then hybridized
with the second strand indicated in the table.

| Position | 1 | 2 | 3 | 4 | 5 | 6 | 7 | 8 | 9 | 10 |
|---|---|---|---|---|---|---|---|---|---|---|
| 1 | AA | AA | AG | AG | AT | AT | CC | CC | AC | AC |
| 2 | CT | CT | ins + 1T | ins + 1T | ins + 2T | ins + 2T | TT | TT | GT | GT |
| 3 | GG | GG | GT | GT | GG | GG | ins + 3T | ins + 3T | ssDNA | ssDNA |
| 4 | TT | TT | AC | AC | AT | AT | AG | AG | ins + 3T | ins + 3T |
| 5 | ssDNA | ssDNA | CC | CC | AA | AA | CT | CT | ins + 2T | ins + 2T |
| 6 | Neg. | Neg. | Neg. | Neg. | AG | AG | GG | GG | ins + 1T | ins + 1T |
| 7 | ssDNA | ssDNA | AT | AT | CC | CC | AC | AC | GT | GT |
| 8 | ins + 3T | ins + 3T | AG | AG | TT | TT | AA | AA | CT | CT |
| 9 | ins + 2T | ins + 2T | AA | AA | AC | AC | AT | AT | CC | CC |
| 10 | GG | GG | CT | CT | ins + 1T | ins + 1T | GT | GT | TT | TT |

TABLE 10
Measuring the red fluorescence intensity on an agarose chip for detecting a
binding of Cy ™ 5-labeled E. coli mutS to DNA double strands
containing different mispairings. The table shows the positions on the chip
together with the appurtenant fluorescence intensities.

| Position | 1 | 2 | 3 | 4 | 5 | 6 | 7 | 8 | 9 | 10 |
|---|---|---|---|---|---|---|---|---|---|---|
| 1 | 54.270 | 60.302 | 47.996 | 45.267 | 28.177 | 27.439 | 31.855 | 31.161 | 32.114 | 33.698 |
| 2 | 44.449 | 51.997 | 47.126 | 49.401 | 60.783 | 60.751 | 31.460 | 31.350 | 142.278 | 136.122 |
| 3 | 55.104 | 67.524 | 223.097 | 230.637 | 71.273 | 71.165 | 40.071 | 40.113 | 23.509 | 23.100 |
| 4 | 31.342 | 32.396 | 40.286 | 41.573 | 31.965 | 32.721 | 54.261 | 55.560 | 40.738 | 38.830 |
| 5 | 19.949 | 21.856 | 38.111 | 38.362 | 62.088 | 61.872 | 54.282 | 58.149 | 72.624 | 69.055 |
| 6 | 10.335 | 8.651 | 10.553 | 10.545 | 55.263 | 50.730 | 79.587 | 82.154 | 56.831 | 62.446 |
| 7 | 20.185 | 22.508 | 36.780 | 35.955 | 39.533 | 40.741 | 44.136 | 48.339 | 289.282 | 297.211 |
| 8 | 35.316 | 39.818 | 59.806 | 62.236 | 41.745 | 40.557 | 77.035 | 82.847 | 75.609 | 77.972 |
| 9 | 62.992 | 73.190 | 74.074 | 80.814 | 57.718 | 55.435 | 42.800 | 43.754 | 54.067 | 54.988 |
| 10 | 67.814 | 84.352 | 67.884 | 71.548 | 75.812 | 74.946 | 330.671 | 287.053 | 49.426 | 61.226 |

TABLE 11
Statistical analysis of the results from the agarose chip containing
different base mispairings. The mean values and standard
deviations for the fluorescence intensities at all the positions with the
same loading were calculated in each case. In addition, the quotient
of the corresponding mean value and the value obtained using perfectly
paired DNA (“AT”) were determined for each mispairing.

| | Mean value +/− | Mispairing/AT |
|---|---|---|
| | standard deviation | quotient |
| Perfect pairing (AT) | 34.9 +/− 6.1 | 1.0 |
| AA mispairing | 69.2 +/− 10.8 | 2.0 |
| AC mispairing | 44.2 +/− 9.3 | 1.3 |
| AG mispairing | 53.9 +/− 5.7 | 1.5 |
| CC mispairing | 41.1 +/− 9.0 | 1.2 |
| CT mispairing | 62.7 +/− 12.2 | 1.8 |
| GG mispairing | 72.4 +/− 9.5 | 2.1 |
| GT mispairing | 242 +/− 72.5 | 6.9 |
| TT mispairing | 39.9 +/− 10.8 | 1.1 |
| Insertion +1T | 61.1 +/− 12.3 | 1.8 |
| Insertion +2T | 66.6 +/− 5.8 | 1.9 |
| Insertion +3T | 39.1 +/− 2.0 | 1.1 |

FIG. 11 shows the binding of Cy™5-labeled E.coli mutS to mispaired DNA double strands which were produced by hybridizing on an electronically addressable agarose chip. In each case, the figure depicts the mean red fluorescence intensity, together with standard deviation, for the different base mispairings and insertions.

It is evident from Table 11 that, in the case of all the mispairings and insertions tested, the mean values for the fluorescence intensities are greater than the value obtained with the perfectly paired double strand. The GT mispairing is the one which is most efficiently bound by mutS; the fluorescence values which are measured at these positions are higher by a factor of about 7 than the values measured in the case of the perfectly paired DNA. On the other hand, mutS only recognizes the CC and TT mispairings weakly. In addition to the different base mispairings, the method described here can also be used to detect insertions of one or two bases (FIG. 11).

### Example: Using mutS to Detect Point Mutations on an Electronically Addressable Hydrogel Chip

The experiment described in the pr vious section for using mutS to recognize different point mutations was rep ated using another type of electronically addressable chip supplied by Nanogen, which chip contained a hydrogel matrix, with streptavidin mol cules mbedded in it, in place of the agarose layer. In order to test which type of chip surface is best suited for the method for recognizing point mutations which is presented here, the results obtained with the two chip types were then compared with each other.

Experimental Implementation:

The hydrogel chip was loaded using the protocol described in the example entitled “the use of mutS to recognize different base mispairings”; however, both the addressing of the usensen first strand oligonucleotide on the hydrogel chip and the hybridization with the different second strands were carried out at a voltage of 2.1 V. The loading scheme is shown in Table 9.

The loaded hydrogel chip was saturated with BSA, incubated with Cy™5-labeled E.coli mutS, and washed, in accordance with the protocol given in the section entitled “the use of mutS to recognize different base mispairings”. The mutS protein which was bound to the individual positions was then detected by measuring the red fluorescence intensity. The same appliance settings were used for this as were used for measuring the agarose chip (“high gain”), integration time, 256 μs). The relative fluorescence intensities which were measured are given in Table 12; the results of the statistical analysis of the measurement data are shown in Table 13 and in FIG. 12.

TABLE 12
Measuring the red fluorescence intensity of a hydrogel chip for detecting
the binding of Cy ™ 5-labeled E. coli mutS to DNA double
strands containing different mispairings. The table gives the positions on the
chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 315.435 | 308.354 | 219.836 | 200.612 | 120.170 | 135.821 | 175.420 | 180.365 | 230.367 | 280.155 |
| 2 | 269.042 | 236.114 | 232.480 | 224.811 | 259.691 | 283.890 | 184.588 | 173.959 | >1049 | >1049 |
| 3 | 458.058 | 448.311 | >1049 | >1049 | 430.176 | 465.028 | 198.013 | 183.445 | 156.958 | 189.712 |
| 4 | 187.157 | 184.343 | 227.621 | 224.721 | 146.877 | 159.355 | 260.817 | 267.459 | 193.354 | 183.243 |
| 5 | 188.130 | 179.783 | 184.442 | 176.890 | 325.214 | 277.688 | 259.714 | 274.394 | 311.466 | 328.294 |
| 6 | 14.785 | 14.977 | 15.611 | 17.457 | 270.687 | 251.188 | 443.439 | 470.905 | 266.818 | 302.247 |
| 7 | 184.799 | 180.179 | 161.906 | 163.021 | 177.822 | 185.842 | 235.587 | 241.388 | >1049 | >1049 |
| 8 | 163.186 | 162.629 | 266.150 | 264.462 | 188.509 | 186.462 | 385.546 | 380.753 | 303.518 | 311.406 |
| 9 | 329.954 | 328.856 | 368.694 | 392.414 | 281.183 | 299.486 | 177.789 | 173.534 | 202.234 | 209.262 |
| 10 | 529.414 | 558.435 | 345.796 | 341.320 | 374.800 | 373.259 | >1049 | >1049 | 220.719 | 228.398 |

TABLE 13
Statistical analysis of the results obtained with the hydrogel chip
containing different mispairings. The mean values and standard
deviations for the fluorescence intensities of all the positions with
the same loading were calculated in each case. The quotient of the
corresponding mean value and the value obtained with
perfectly paired DNA were also determined for each mispairing.

| | Mean value +/− | Mispairing/AT |
|---|---|---|
| | standard deviation | quotient |
| Perfect pairing (AT) | 154.8 +/− 19.4 | 1.0 |
| AA mispairing | 344.3 +/− 42.9 | 2.2 |
| AC mispairing | 252.6 +/− 29.5 | 1.6 |
| AG mispairing | 250.2 +/− 25.8 | 1.6 |
| CC mispairing | 186.5 +/− 12.5 | 1.2 |
| CT mispairing | 292.7 +/− 39.3 | 1.9 |
| GG mispairing | 475.5 +/− 44.8 | 3.1 |
| GT mispairing | >1049 | >6.7 |
| TT mispairing | 194.3 +/− 19.3 | 1.3 |
| Insertion +1T | 295.7 +/− 66.6 | 1.9 |
| Insertion +2T | 307.0 +/− 29.2 | 2.0 |
| Insertion +3T | 180.6 +/− 14.9 | 1.2 |

FIG. 12 shows the binding of Cy™5-labeled E.coli mutS to mispaired DNA double strands which were produced by hybridizing on an electronically addressable hydrogel chip. In each case, the figure shows the mean red fluorescence intensity, with standard deviation, for the different base mispairings and insertions.

As is evident from Table 13 and FIG. 12, a similar picture is obtained when using the hydrogel chip as was obtained with the agarose chip: The GT mispairing is the one which is most efficiently recognized by E.coli mutS, whereas DNA double strands containing the CC and TT mispairings are hardly bound any more strongly by the protein than are perfectly paired double strands. However, all in all, the quotients between the fluorescence values obtained with mispaired DNA and with perfectly paired DNA are somewhat higher in the case of the hydrogel chip (Table 13) than in the case of the agarose chip (Table 11). It is therefore possible to obtain a better distinction between mutated and non-mutated DNA when the hydrogel chip is used. In addition to this, when the measuring instrument is adjusted to the same setting of highest sensitivity (“high gain”), the absolute values which are measured in the case of the hydrogel chip are higher by a factor of 4-5 than those obtained with the agarose chip, thereby making it possible to exploit the measurement range more efficiently. In the case of the hydrogel chip, the fluorescence intensity obtained with the GT mispairing is even in the saturation range (>1049). A possible explanation for the higher fluorescence on the hydrogel chip is that the relatively large mutS protein is better able to penetrate into the pores of the hydrogel layer than into the agarose matrix.

In summary, it was possible to demonstrate, by making the comparison between the agarose chip and the hydrogel chip, that both types of chip are suitable for the mutation detection based on mutS. However, as compared with the agarose chip, the hydrogel chip offers the advantages of higher sensitivity and better discrimination between mispaired and perfectly paired DNA.

### Comparison Example: Recognizing Base Mispairing Using Dye-Labeled mutS Proteins

The binding of mutS proteins to a variety of base mispairings was investigated using surface plasmon resonance technology. This was necessary in order to check whether all base mispairings are indeed specifically bound by the proteins. In contrast to the band shift assay which has already been described, surface plasmon resonance technology enables the binding events to be quantified more precisely.

The 'sense′ oligonucleotide which is biotinylated at the 5′ end (Seq. ID No. 11), and oligonucleotides which are partially complementary to this oligonucleotide (Seq. ID No. 12-22), were synthesized for this purpose (Biospring, Frankfurt/Main). If these latter oligonucleotides are hybridized against the usenseu oligonucleotide, this then results in the formation of completely pared double-stranded DNA (AT), double-stranded DNA containing a base mispairing (AA, AC, AG, CC, CT, GG, GT, TT) or double-stranded DNA containing an insertion of 1, 2 or 3 thymidine residues (Table 14).

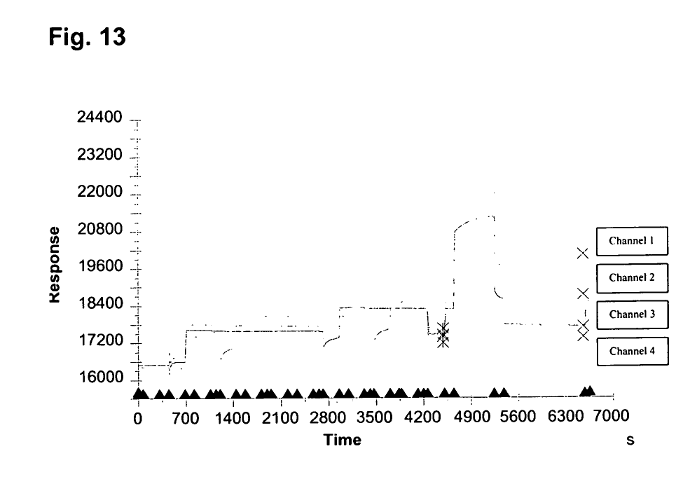

The oligonucleotides were taken up, to a concentration of 2 pmol/μl, in HBS-EP buffer (10 mM HEPES pH 7.4, 150 mM NaCl, 3 mM EDTA, 0.005% (w/v) polysorbate20, 5 mM MgCl₂). The usensen oligonucleotide was then applied, at a flow rate of 5 μl/min, to the streptavidin-loaded channels of the surface of a biacore SA chip until saturation was achieved, as shown, by way of example, in FIG. 13. The oligonucleotides which were partially complementary to this oligonucleotide were applied, under identical buffer conditions, to the respective channels of the chip, as in the example “using mutS to detect point mutations on an electronically addressable hydrogel chip”, in order to obtain the double-stranded DNA species depicted in Table 14. After the double-stranded oligonucleotides had been introduced, the chip surface was equilibrated with 20 mM tris-HCl pH 7.6, 50 mM KCl, 5 mM MgCl₂, 0.01 % Tween-20, 10% (v/v) glycerol. The Cy™5-labeled mutS-maltose binding protein fusion protein was taken up, to a concentration of 0.1 μg/μl, in the same buffer and loaded onto the chip at a flow rate of 5 μul/min. The channels of the chip were subsequently rinsed with 100 μl of the buffer 20 mM tris-HCl pH 7.6, 50 mM KCl, 5 mM MgCl₂, 0.01 % Tween-20, 10% (v/v) glycerol, resulting in nonspecifically bound protein being washed away. After the rinsing, the quantity of protein (expressed as a resonance unit) which was bound to each respective double-stranded oligonucleotide was determined (Table 14). When this was done, it was found that, in principle, the labeled fusion protein bound better to all the base mispairings and insertions, apart from the CC base mispairing, than it did to the perfectly paired “AT” oligonucleotide (Table 14). Consequently, the fluorescent dye-labeled mutS protein which we conceived and prepared is able to locate any possible mutation. The authors are not aware of any other fluorescence dye-labeled protein whose DNA-binding properties remain preserved after the labeling, as is the case with the protein which is described here.

FIG. 13 shows an example of a sensogram of the mutS binding. This sensogram depicts the chronological change in the mass (RU, resonance units) in the 4 channels of a Biacore-SA chip. The biotinylated “sense” oligonucleotide was applied to channels 1-4 and hybridized with the respective counterstrands in order to produce a perfectly paired double strand (“AT”, channel 4) and DNA containing the GT (channel 1), CC (channel 3) and GG (channel 2) base mispairings. The protein was then loaded on (from t=4638 to t=5239). Unbound protein was removed by washing (from t=5378 to to t=6579). The resonance prior to the protein loading (t=4502) was subtracted from the resonance which was measured after the rinsing (t=6579). The difference corresponds to the mass of the bound mutS protein.

TABLE 14

The table shows the binding of the MBP-mutS fusion protein to double stranded
oligonucleotides which are bound to the surface of Biacore ™ SA chips and which
contain base mispairings (underlined bases).

| | | |
|---|---|---|
| Base | | |
| mispair- | | |
| ing/in- | | Resonance |
| serion | Double-stranded oligonucleotide (upper strand in the 5′ > 3′ direction) | units |

| | | |
|---|---|---|
| AA | 5′-AAG CAT ACG GAA GTT AAA GTG CGG ATC ATC TCT AGC-3′ | 973.6 |
| | 3′-C GTA TGC CTT CAA TTA CAC GCC TAG TAG AGA TCG GT-5′ | |
| AC | 5′-AAG CAT ACG GAA GTT AAA GTG CGG ATC ATC TCT AGC-3′ | 988.5 |
| | 3′-C GTA TGC CTT CAA TTC CAC GCC TAG TAG AGA TCG GT-5′ | |
| AG | 5′-AAG CAT ACG GAA GTT AAA GTG CGG ATC ATC TCT AGC-3′ | 444.6 |
| | 3′-C GTA TGC CTT CAA TGT CAC GCC TAG TAG AGA TCG GT-5′ | |
| AT | 5′-AAG CAT ACG GAA GTT AAA GTG CGG ATC ATC TCT AGC-3′ | 272.6 |
| | 3′-C GTA TGC CTT CAA TTT CAC CCC TAG TAG AGA TCG GT-5′ | |
| CC | 5′-AAG CAT ACG GAA GTT AAA GTG CGG ATC ATC TCT AGC-3′ | 252.4 |
| | 3′-C GTA TGC CTT CAA TTT CAC CCC TAG TAG AGA TCG GT-5′ | |
| CT | 5′-AAG CAT ACG GAA GTT AAA GTG CGG ATC ATC TCT AGC-3′ | 73.6 |
| | 3′-C GTA TGC CTT CAA TTT CCC GCC TAG TAG AGA TCG GT-5′ | |
| GG | 5′-AAG CAT ACG GAA GTT AAA GTG CGG ATC ATC TCT AGC-3′ | 1098.8 |
| | 3′-C GTA TGC CTT CAA TTT CAG GCC TAG TAG AGA TCG GT-5′ | |
| GT | 5′-AAG CAT ACG GAA GTT AAA GTG CGG ATC ATC TCT AGC-3′ | 832.12 |
| | 3′-C GTA TGC CTT CAA TTT CGC GCC TAG TAG AGA TCG GT-5′ | |
| TT | 5′-AAG CAT ACG GAA GTT AAA GTG CGG ATC ATC TCT AGC-3′ | 71.1 |
| | 3′-C GTA TGC CTT CAA TTT CTC GCC TAG TAG AGA TCG GT-5′ | |
| +1T | 5′-AAG CAT ACG GAA GTT AAA GTG CGG ATC ATC TCT AGC-3′ | 126.8 |
| | 3′-C GTA TGC CTT CAA TTT TCA CGC CTA GTA GAG ATC GGT-5′ | |
| +2T | 5′-AAG CAT ACG GAA GTT AAA GTG CGG ATC ATC TCT AGC-3′ | 1958.6 |
| | 3′-C GTA TGC CTT CAA TTT TTC ACG CCT AGT AGA GAT CGG T-5′ | |

### Example: Detecting GT Mispairings in Different Oligonucleotides

It is reported in the literature that the recognition of point mutations by mutS is not only dependent on the nature of the base mispairing which has been formed but is also influenced by the nucleotide sequence in the environment of the mispairing (M. Jones et al., Genetics 115 (1987), 505-610). A test was therefore carried out to determine whether it is possible to use the method which is described here to reliably detect GT mispairings independently of the particular sequence context. For this experiment, eight different first-strand oligonucleotides having different base sequences were addressed to defined positions on an agarose chip and on a hydrogel chip and hybridized in each case with the complementary counterstrands for perfect pairing (“AT”) and for GT mispairing. The binding of E.coli-mutS to the different double strands was then investigated.

Experimental implementation: All the oligonucleotides were dissolved, at a concentration of 100 nM, in histidine buffer and denatured at 95° C. for 5 min. The biotinylated first-strand “sense” oligonucleotide (Seq. ID No. 10), APC se (Seq. ID No. 24), bcl se (Seq. ID No. 27), Brc se (Seq. ID No. 30), Met se (Seq. ID No. 33), MSH se (Seq. ID No. 36), p53 se (Seq. ID No. 39) and Rb se (Seq. ID No. 42) were electronically addressed to the individual positions in the Nanogen workstation loading appliance for 60 sec at a voltage of 2.0 V (in the case of the agarose chip) and of 2.1 V (in the case of the hydrogel chip). The hybridization with the Cy™3-labeled counterstrands was carried out for 120 sec at 2.0 V (in the case of the agarose chip) and at 2.1 V (in the case of the hydrogel chip). The loading scheme for the agarose chip is depicted in Table 15 while the loading scheme for the hydrogel chip is depicted in Table 19. The following second-strand oligonucleotides were used:

for perfect pairing: AT (Seq. ID No. 12), APC AT (Seq. ID No. 25), bcl AT (Seq. ID No. 28), Brc AT (Seq. ID No. 31), Met AT (Seq. ID No. 34), MSH AT (Seq. ID No. 37), p53 AT (Seq. ID No. 40), Rb AT (Seq. ID No. 43)

for GT mispairing: GT (Seq. ID No. 13), APC GT (Seq. ID No. 26), bcl GT (Seq. ID No. 29), Brc GT (Seq. ID No. 32), Met GT (Seq. ID No. 35), MSH GT (Seq. ID No. 38), p53 GT (Seq. ID No. 41), Rb GT (Seq. ID No. 44)

After the loading, the chips were taken out of the loading appliance, filled with 1 ml of blocking buffer and incubated at room temperature for 60 min in order to saturate nonspecific protein-binding sites. The chips were then incubated, at room temperature for 60 min, with 10 μl of Cy™5-labeled E. coli mutS (concentration: 50 ng/μl) in 90 μl of incubation buffer. After this incubation, the chips were washed by hand with 1 ml of incubation buffer, then inserted into the Nanogen reader and washed in the reader, at a temperature of 37° C., 70× with in each case 0.5 ml of washing buffer.

Finally, the Cy™5 and Cy™3 fluorescence intensities were measured at the individual positions on the chip in the Nanogen reader using the following instrument settings:

Red fluorescence (Cy™5): high sensitivity (“high gain”); 256 μs integration time

Green fluorescence (Cy™3): low sensitivity (“low gain”); 256 μs integration time

Histidine buffer: 50 mM L-histidine; this solution was filtered through a membrane having a pore size of 0.2 μm and degassed by negative pressure.

Blocking buffer: 20 mM tris, pH 7.6/50 mM KCl/5 mM MgCl₂/0.01% Tween-20/3% BSA

Incubation buffer: 20 mM tris, pH 7.6/50 mM KCl/5 mM MgCl₂/0.01% Tween-20/1% BSA

Washing buffer: 20 mM tris, pH 7.6/50 mM KCl/5 mM MgCl₂/0.1% Tween-20

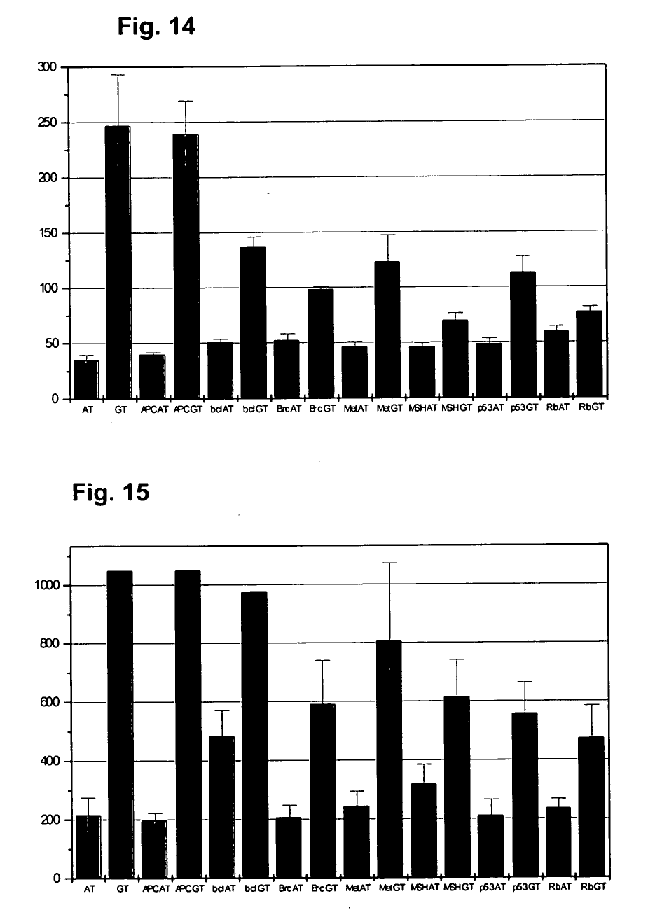

In the case of the experiment which was carried out on an agarose chip, the green fluorescence intensities which were measured are given in Table 16 while the red fluorescence intensities are given in Table 17. In addition to the positions on the chip which, as negative controls, had not been loaded with DNA, some other compartments (positions 1/10, 2/4, 2/5, 2/6, 2/10 and 3/2) also only exhibited slight green fluorescence. Since the loading with the Cy™3-labeled second strand had presumably not functioned at these positions, they were not included in the further analysis. The results of the statistical analysis of the red fluorescence intensities are depicted in Table 18 and in FIG. 14.

In the case of the experiment carried out on a hydrogel chip, the green fluorescence intensities which were measured are listed in Table 20 while the red fluorescence intensities are listed in Table 21. The green fluorescence intensities were all lower than in the case of the agarose chip. The results of the statistical analysis of the red fluorescence intensities are depicted in Table 22 and in FIG. 15. Because of the very low green fluorescence, chip position 9/6 was not included in the analysis.

TABLE 15
Scheme for loading an agarose chip for detecting the binding of Cy ™ 5-labeled E. coli mutS to GT
base mispairings in different DNA double strands. Empty boxes symbolize positions which are not loaded with
DNA. All the remaining positions were first of all addressed with the oligonucleotide named in the upper line
and, after that, hybridized with second strand given in the lower line.

| Position | 1 | 2 | 3 | 4 | 5 | 6 | 7 | 8 | 9 | 10 |
|---|---|---|---|---|---|---|---|---|---|---|
| 1 | sense | sense | sense | p53 se | p53 se | p53 se | APC se | APC se | APC se | bcl se |
| | AT | AT | AT | p53 AT | p53 AT | p53 AT | APC AT | APC AT | APC AT | bcl AT |
| 2 | sense | sense | sense | p53 se | p53 se | p53 se | APC se | APC se | APC se | bcl se |
| | GT | GT | GT | p53 GT | p53 GT | p53 GT | APC GT | APC GT | APC GT | bcl GT |
| 3 | bcl se | bcl se | Brc se | Brc se | Brc se | Met se | Met se | Met se | MSH se | MSH se |
| | bcl AT | bcl AT | Brc AT | Brc AT | Brc AT | Met AT | Met AT | Met AT | MSH | MSH |
| | | | | | | | | | AT | AT |
| 4 | bcl se | bcl se | Brc se | Brc se | Brc se | Met se | Met se | Met se | MSH se | MSH se |
| | bcl GT | bcl GT | BrcGT | Brc GT | Brc GT | Met GT | Met GT | Met GT | MSH | MSH |
| | | | | | | | | | GT | GT |
| 5 | MSH se | | Rb se | Rb se | Rb se | sense | sense | sense | p53 se | p53 se |
| | MSH | | Rb AT | Rb AT | Rb AT | AT | AT | AT | p53 AT | p53 AT |
| | AT | | | | | | | | | |
| 6 | MSH se | | Rb se | Rb se | Rb se | sense | sense | sense | p53 se | p53 se |
| | MSH | | Rb GT | Rb GT | Rb GT | GT | GT | GT | p53 GT | p53 GT |
| | GT | | | | | | | | | |
| 7 | P53 se | APC se | APC se | APC se | bcl se | bcl se | bcl se | Brc se | Brc se | Brc se |
| | p53 AT | APC AT | APC AT | APC AT | bcl AT | bcl AT | bcl AT | Brc AT | Brc AT | Brc AT |
| 8 | P53 se | APC se | APC se | APC se | bcl se | bcl se | bcl se | Brc se | Brc se | Brc se |
| | p53 GT | APC GT | APC GT | APC GT | bcl GT | bcl GT | bcl GT | Brc GT | Brc GT | Brc GT |
| 9 | Met se | Met se | Met se | MSH se | MSH se | MSH se | Rb se | Rb se | Rb se | |
| | Met AT | Met AT | Met AT | MSH | MSH | MSH | Rb AT | Rb AT | Rb AT | |
| | | | | AT | AT | AT | | | | |
| 10 | Met se | Met se | Met se | MSH se | MSH se | MSH se | Rb se | Rb se | Rb se | |
| | Met GT | Met GT | Met GT | MSH | MSH | MSH | Rb GT | Rb GT | Rb GT | |
| | | | | GT | GT | GT | | | | |

TABLE 16
Measuring the green fluorescence intensity on an agarose chip in order to check
the loading with Cy ™ 3-labeled second-strand oligonucleotides.
The table gives the positions on the chip together with the appurtenant relative
fluorescence intensities.

| Position | 1 | 2 | 3 | 4 | 5 | 6 | 7 | 8 | 9 | 10 |
|---|---|---|---|---|---|---|---|---|---|---|
| 1 | >1049 | >1049 | >1049 | >1049 | 899.689 | >1049 | >1049 | >1049 | >1049 | 5.007 |
| 2 | >1049 | >1049 | >1049 | 14.828 | 13.138 | 31.877 | >1049 | >1049 | >1049 | 71.457 |
| 3 | 898.395 | 422.918 | >1049 | >1049 | >1049 | >1049 | >1049 | >1049 | >1049 | >1049 |
| 4 | >1049 | 943.621 | >1049 | >1049 | >1049 | >1049 | 917.779 | >1049 | >1049 | >1049 |
| 5 | >1049 | 5.507 | >1049 | >1049 | >1049 | >1049 | >1049 | >1049 | 860.318 | 760.914 |
| 6 | >1049 | 5.534 | >1049 | >1049 | >1049 | >1049 | >1049 | >1049 | 835.609 | 779.035 |
| 7 | >1049 | >1049 | 921 .971 | >1049 | 903.584 | 715.283 | 907.024 | >1049 | >1049 | >1049 |
| 8 | >1049 | >1049 | >1049 | >1049 | >1049 | 866.510 | >1049 | >1049 | >1049 | >1049 |
| 9 | >1049 | 813.733 | >1049 | >1049 | 905.773 | >1049 | >1049 | >1049 | >1049 | 5.116 |
| 10 | 817.662 | 769.310 | 921.571 | >1049 | 874.443 | >1049 | >1049 | >1049 | >1049 | 4.768 |

TABLE 17
Measuring the red fluorescence intensity on an agarose chip in order to detect
the binding of Cy ™ 5-labeled E. coli mutS to GT base mispairings
in different DNA double strands. The table gives the positions on
the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 29.227 | 32.877 | 32.354 | 46.589 | 44.844 | 47.260 | 39.466 | 38.975 | 40.713 | 18.817 |
| 2 | 178.176 | 209.865 | 238.731 | 30.084 | 28.736 | 33.106 | 269.490 | 262.903 | 190.954 | 41.498 |
| 3 | 47.766 | 36.567 | 51.707 | 46.607 | 45.575 | 47.066 | 48.706 | 52.239 | 50.715 | 43.733 |
| 4 | 122.012 | 137.030 | 93.469 | 97.895 | 99.722 | 136.525 | 137.084 | 154.376 | 80.076 | 74.767 |
| 5 | 40.345 | 12.897 | 54.121 | 56.429 | 57.908 | 37.381 | 38.421 | 40.765 | 55.122 | 54.313 |
| 6 | 60.014 | 12.111 | 75.309 | 79.893 | 76.602 | 286.125 | 277.349 | 291.828 | 123.875 | 118.537 |
| 7 | 43.860 | 41.807 | 41.897 | 40.384 | 51.963 | 51.865 | 53.154 | 54.797 | 55.288 | 60.511 |
| 8 | 96.606 | 222.345 | 239.863 | 253.370 | 145.452 | 138.379 | 141.868 | 96.068 | 99.637 | 100.641 |
| 9 | 40.248 | 41.906 | 47.720 | 45.862 | 46.015 | 48.560 | 62.053 | 59.498 | 67.663 | 17.127 |
| 10 | 89.180 | 107.020 | 118.011 | 67.897 | 66.284 | 67.525 | 75.611 | 72.230 | 84.991 | 14.945 |

TABLE 18
Statistical analysis of the agarose chip containing different
perfectly paired and GT-mispaired DNA double strands.
The mean values and standard deviations of the red
fluorescence intensities at all positions having the same loading
were calculated in each case. In addition, the quotient of the
fluorescence obtained after adding the corresponding
GT-mispairing second strand and the fluorescence obtained
after adding the completely complementary second strand was
determined for each first strand. Because of their low level of
green fluorescence, positions 1/10, 2/4, 2/5, 2/6, 2/10 and
3/2 were not included in the analysis.

| | Perfect pairing | | GT/AT |
|---|---|---|---|
| First strand | (AT) | GT mispairing | quotient |
| Sense | 35.2 +/− 4.4 | 247.0 +/− 46.1 | 7.0 |
| APC se | 40.5 +/− 1.2 | 239.8 +/− 29.3 | 5.9 |
| bcl se | 51.2 +/− 2.4 | 136.9 +/− 9.0 | 2.7 |
| Brc se | 52.4 +/− 5.7 | 97.9 +/− 2.7 | 1.9 |
| Met se | 46.3 +/− 4.5 | 123.7 +/− 23.6 | 2.7 |
| MSH se | 45.9 +/− 3.6 | 69.4 +/− 7.0 | 1.5 |
| p53 se | 48.7 +/− 4.8 | 113.0 +/− 14.5 | 2.3 |
| Rb se | 59.6 +/− 4.8 | 77.4 +/− 4.4 | 1.3 |

FIG. 14 shows the binding of Cy™5-labeled E.coli mutS to different perfectly paired (AT) and GT-mispaired DNA double strands which were produced by hybridizing on an electronically addressable agarose chip. The figure in each case depicts the mean red fluorescence intensity, together with standard deviation, for the different double strands.

TABLE 19
Scheme for loading a hydrogel chip for detecting the binding Cy ™ 5-labeled E. coli mutS to GT
base mispairings in different DNA double strands. Empty boxes symbolize positions which were not loaded
with DNA. All the remaining positions were firstly addressed with the oligonucleotide named in the upper line
and, after that, hybridized with the second strand given in the lower line.

| Position | 1 | 2 | 3 | 4 | 5 | 6 | 7 | 8 | 9 | 10 |
|---|---|---|---|---|---|---|---|---|---|---|
| 1 | sense | sense | sense | p53 se | p53 se | p53 se | APC se | APC se | APC se | bcl se |
| | AT | AT | AT | p53 AT | p53 AT | p53 AT | APC AT | APC AT | APC AT | bcl AT |
| 2 | sense | sense | sense | p53 se | p53 se | p53 se | APC se | APC se | APC se | bcl se |
| | GT | GT | GT | p53 GT | p53 GT | p53 GT | APC GT | APC GT | APC GT | bcl GT |
| 3 | bcl se | bcl se | Brc se | Brc se | Brc se | Met se | Met se | Met se | MSH se | MSH se |
| | bcl AT | bcl AT | Brc AT | Brc AT | Brc AT | Met AT | Met AT | Met AT | MSH | MSH |
| | | | | | | | | | AT | AT |
| 4 | bcl se | bcl se | Brc se | Brc se | Brc se | Met se | Met se | Met se | MSH se | MSH se |
| | bcl GT | bcl GT | BrcGT | Brc GT | Brc GT | Met GT | Met GT | Met GT | MSH | MSH |
| | | | | | | | | | GT | GT |
| 5 | MSH se | MSH se | Rb se | Rb se | Rb se | sense | sense | sense | p53 se | p53 se |
| | MSH | MSH | Rb AT | Rb AT | Rb AT | AT | AT | AT | p53 AT | p53 AT |
| | AT | AT | | | | | | | | |
| 6 | MSH se | | Rb se | Rb se | Rb se | sense | sense | sense | p53 se | p53 se |
| | MSH | | Rb GT | Rb GT | Rb GT | GT | GT | GT | p53 GT | p53 GT |
| | GT | | | | | | | | | |
| 7 | p53 se | APC se | APC se | APC se | bcl se | bcl se | bcl se | Brc se | Brc se | Brc se |
| | p53 AT | APC AT | APC AT | APC AT | bcl AT | bcl AT | bcl AT | Brc AT | Brc AT | Brc AT |
| 8 | p53 se | APC se | APC se | APC se | bcl se | bcl se | bcl se | Brc se | Brc se | Brc se |
| | p53 GT | APC GT | APC GT | APC GT | bcl GT | bcl GT | bcl GT | Brc GT | Brc GT | Brc GT |
| 9 | Met se | Met se | Met se | MSH se | | MSH se | Rb se | Rb se | Rb se | |
| | Met AT | Met AT | Met AT | MSH | | MSH | Rb AT | Rb AT | Rb AT | |
| | | | | AT | | AT | | | | |
| 10 | Met se | Met se | Met se | MSH se | MSH se | MSH se | Rb se | Rb se | Rb se | |
| | Met GT | Met GT | Met GT | MSH | MSH | MSH | Rb GT | Rb GT | Rb GT | |
| | | | | GT | GT | GT | | | | |

TABLE 20
Measuring the green fluorescence intensity of the hydrogel chip for checking
the loading with Cy ™ 3-labeled second-strand oligonucleotides.
The table gives the positions on the chip together with the appurtenant
relative fluorescence intensities.

| Position | 1 | 2 | 3 | 4 | 5 | 6 | 7 | 8 | 9 | 10 |
|---|---|---|---|---|---|---|---|---|---|---|
| 1 | 715.700 | 710.008 | 749.977 | 547.095 | 458.894 | 518.614 | 604.708 | 509.088 | 616.297 | 221.316 |
| 2 | 674.979 | 755.446 | 759.574 | 462.223 | 358.411 | 462.312 | 631.912 | 511.214 | 626.089 | 420.006 |
| 3 | 602.724 | 666.304 | 786.049 | 590.611 | 707.440 | 243.454 | 211.541 | 232.284 | 531.493 | 483.863 |
| 4 | 719.978 | 793.798 | 643.035 | 504.802 | 411.324 | 272.219 | 251.396 | 364.279 | 217.965 | 254.634 |
| 5 | 466.434 | 506.635 | 759.000 | 694.698 | 681.840 | 717.489 | 728.218 | 800.407 | 723.757 | 764.338 |
| 6 | 539.375 | 4.980 | 518.676 | 467.897 | 539.331 | 707.519 | 689.322 | 753.603 | 496.537 | 457.917 |
| 7 | 850.324 | 634.120 | 464.043 | 635.432 | 499.300 | 412.674 | 553.598 | 668.613 | 598.881 | 511.598 |
| 8 | 937.970 | 622.288 | 493.267 | 641.694 | 463.264 | 419.173 | 568.347 | 705.419 | 672.772 | 592.863 |
| 9 | 211.967 | 243.571 | 270.525 | 708.729 | 4.920 | 5.099 | 697.343 | 649.921 | 670.883 | 4.804 |
| 10 | 219.665 | 202.494 | 270.224 | 508.069 | 483.704 | 498.981 | 447.416 | 414.754 | 452.484 | 4.619 |

TABLE 21
Measuring the red fluorescence intensity on a hydrogel chip for detecting
the binding of Cy ™ 5-labeled E. coli mutS to GT base mispairings
in different DNA double strands. The table gives the positions on
the chip together with the appurtenant fluorescence intensities.

| Position | 1 | 2 | 3 | 4 | 5 | 6 | 7 | 8 | 9 | 10 |
|---|---|---|---|---|---|---|---|---|---|---|
| 1 | 308.405 | 259.552 | 220.679 | 198.480 | 158.286 | 148.395 | 166.251 | 171.246 | 206.122 | 623.586 |
| 2 | >1049 | >1049 | >1049 | 519.216 | 488.602 | 508.844 | >1049 | >1049 | >1049 | 780.898 |
| 3 | 555.731 | 411.186 | 195.120 | 155.951 | 158.852 | 157.645 | 242.199 | 228.068 | 223.005 | 315.212 |
| 4 | >1049 | 872.005 | 505.932 | 434.103 | 458.503 | 537.876 | 570.664 | 576.150 | 409.264 | 525.467 |
| 5 | 425.414 | 342.152 | 207.796 | 202.840 | 206.352 | 162.356 | 161.744 | 188.192 | 226.610 | 243.972 |
| 6 | 649.081 | 37.142 | 405.147 | 330.908 | 391.253 | >1049 | >1049 | >1049 | 542.130 | 526.798 |
| 7 | 292.335 | 229.174 | 219.451 | 188.552 | 397.595 | 443.787 | 460.836 | 242.999 | 230.846 | 250.774 |
| 8 | 769.070 | >1049 | >1049 | >1049 | >1049 | >1049 | >1049 | 646.316 | 687.545 | 813.315 |
| 9 | 272.200 | 277.027 | 295.230 | 289.986 | 61.106 | 43.935 | 257.158 | 249.294 | 281.313 | 33.565 |
| 10 | >1049 | >1049 | >1049 | 692.481 | 732.107 | 693.430 | 584.899 | 565.577 | 566.162 | 41.500 |

TABLE 22
Statistical analysis of the hydrogel chip containing different
perfectly paired and GT-mispaired DNA double strands.
The mean values and standard deviations of the red
fluorescence intensities at all the positions having the same
loading were calculated in each case. In addition, the quotient
of the fluorescence following the addition of the corresponding
GT-mispairing second strand and the fluorescence following
the addition of the completely complementary second strand
was determined for each first strand. Because of its low level of
green fluorescence, position 9/6 was not included in the analysis.

| | Perfect pairing | | GT/AT |
|---|---|---|---|
| First strand | (AT) | GT mispairing | quotient |
| Sense | 216.8 +/− 58.4 | >1049 | >4.8 |
| APC se | 196.8 +/− 25.7 | >1049 | >5.3 |
| bcl se | 482.1 +/− 88.9 | >974.6 | >2.0 |
| Brc se | 205.8 +/− 42 | 590.9 +/− 149.1 | 2.9 |
| Met se | 245.4 +/− 49.4 | 805.1 +/− 267.1 | 3.3 |
| MSH se | 319.1 +/− 66.2 | 617.0 +/− 124.4 | 1.9 |
| p53 se | 211.3 +/− 54.4 | 559.1 +/− 104.4 | 2.6 |
| Rb se | 234.1 +/− 33.0 | 474.0 +/− 110.7 | 2.0 |

FIG. 15 shows the binding of Cy™5-labeled E.coli mutS to different perfectly paired (AT) and GT-mispaired DNA double strands which were produced by hybridizing on an electronically addressable hydrogel chip. The figure in each case depicts the mean red fluorescence intensity, together with standard deviation, for the different double strands.

The analysis of the experiment showed that it was possible to detect all the tested GT mispairings, both on the agarose chip (Table 18; FIG. 14) and on the hydrogel chip (Table 22; FIG. 15), using the method which is described here: in all cases, the mutS protein bound more strongly to the mispairing than to the respective perfectly paired double strand. It is consequently possible to use mutS to reliably detect GT base mispairings although the quotient between the measured values obtained with GT-mispaired and perfectly paired DNA is certainly affected by the neighboring DNA sequence.

A comparison between the results obtained with the two different chip types (Table 18 and Table 22) shows in this case, too, that better discrimination between perfectly paired DNA and mispaired DNA is obtained on the hydrogel chip than on the agarose chip: in the case of five of the tested sequences, the quotients between the measured values in the case of mispaired and perfectly paired DNA (GT/AT) gave higher values on the hydrogel chip. As far as the remaining three DNA sequences (“sense”, bcl se and APC se) were concerned, the fluorescence measured for the GT mispairing was in the saturation range (>1049) in the case of the hydrogel chip, which meant that it was not possible to determine any reliable value for the quotient in these instances.

### Example: Recognizing GT Mispairings in a Mixture of Perfectly Paired and Mispaired DNA Double Strands

If DNA or cDNA is isolated from a human or animal tissue, the isolated strands do not all always exhibit the same nucleotide sequence. This can result from the fact that the donor organism is heterozygous for a mutation (i.e. in each cell, the mutation is only present on one of the two homologous chromosomes) or to the fact that only some of the cells in the tissue exhibit a particular mutation. This situation frequently occurs in tumors, in particular, since tumor cells are genetically unstable. When such inhomogenous patient DNA is hybridized with a reference DNA, a mixture of mispaired and perfectly paired double strands will then be formed.

In the following experiment, a test was carried out to determine how high the proportion of mispaired DNA has to be in a mixture so as still to ensure that the mutation is detected by the E. coil mutS protein. For this, the “sense” (Seq. ID No. 10) and p53 se (Seq. ID No. 39) first-strand oligonucleotides, which had been addressed onto an agarose chip, were in each case hybridized with different mixtures of perfectly paired and GT-mispaired second strands.

The following mixtures of the AT (Seq. ID No.12) ad GT (S q. ID No. 13) oligonucleotides were used as the second strand for hybridizing with the first-strand “sense” DNA:

AT: Hybridization took place using 100 nM AT

GT: Hybridization took place using 100 nM GT

3%GT: Hybridization took place using a mixture consisting of 3 nM GT+97 nM AT

10%GT: Hybridization took place using a mixture consisting of 10 nM GT+90 nM AT

25%GT: Hybridization took place using a mixture consisting of 25 nM GT+75 nM AT

50%GT: Hybridization took place using a mixture consisting of 50 nM GT+50 nM AT

75%GT: Hybridization took place using a mixture consisting of 75 nM GT+25 nM AT

The corresponding mixtures of the p53 AT (Seq. ID No. 40) and p53 GT (Seq. ID No. 41) oligonucleotides were employed for hybridizing with the p53 se first strand.

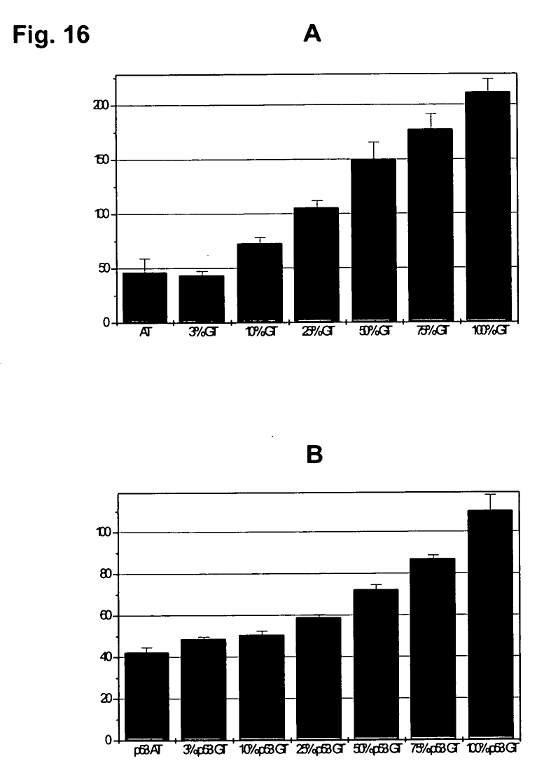

Experimental implementation: The first-strand and second-strand oligonucleotides were dissolved in histidine buffer (total concentration: in each case 100 nM) and denatured at 95° C. for 5 min. The biotinylated “sense” and p53 se oligonucleotides were electronically addressed to individual positions on the Nanogen agarose chip for 60 sec, and at a voltage of 2.0 V, in the Nanogen workstation charging set. The hybridization with the different second-strand mixtures was carried out for 120 sec at 2.0 V. The loading scheme is depicted in Table 23. After the loading, the chip was taken out of the loading appliance, filled with 1 ml of blocking buffer and incubated at room temperature for 60 min in order to saturate nonspecific protein-binding sites. The chip was subsequently incubated, at room temperature for 60 min, with 10 μl of Cy™5-labeled E.coli mutS (concentration: 50 ng/μl) in 90 μl of incubation buffer. After this incubation, the chip was washed by hand with 1 ml of incubation buffer and then inserted into the Nanogen reader and washed, in the reader and at a temperature of 37° C., 70× with in each case 0.5 ml of washing buffer. Finally, the Cy™5 fluorescence intensities at the individual positions of the chip were measured in the Nanogen reader using the following instrument settings: high sensitivity (“high gain”); 256 μs integration time. The relative fluorescence intensities which were measured are given in Table 24: the results of the statistical analysis of the measurement data are shown in Table 25 and in FIG. 16A and FIG. 16B.

Histidine buffer: 50 mM L-histidine; this solution was filtered through a membrane having a pore size of 0.2 μm and degassed.

Blocking buffer: 20 mM trs, pH 7.6/50 mM KCl/5 mM MgCl₂/0.01% Tween-20/3% BSA

Incubation buffer: 20 mM trs, pH 7.6/50 mM KCl/5 mM MgCl₂/0.01% Tween-20/1% BSA

Washing buffer: 20 mM trs, pH 7.6/50 mM KCl/5 mM MgCl₂/0.1% Tween-20

TABLE 23
Scheme for loading an agarose chip for detecting the binding of Cy ™ 5-labeled E. coli mutS to
different mixtures of perfectly paired and GT-mispaired DNA double strands. Empty boxes symbolize positions
which were not loaded with DNA (negative controls). Boxes with bold lettering identify positions which were
only loaded with one DNA strand (single-stranded controls). All the remaining positions were first of all
addressed with the biotinylated oligonucleotide which is named in the upper line and, after that, hybridized with
the second-strand mixture which is given in the second line. As additional controls, the positions identified by
italic lettering were loaded with a combination of first-strand and a non-complementary second strand; there
should not be any hybridization at these positions.

| | 1 | 2 | 3 | 4 | 5 | 6 | 7 | 8 | 9 | 10 |
|---|---|---|---|---|---|---|---|---|---|---|
| 1 | sense | sense | sense | sense | sense | sense | sense | | | |
| | p53 AT | p53 AT | 75% GT | 50% GT | 25% GT | 10% GT | 3% GT | | | |
| 2 | sense | sense | sense | sense | sense | sense | sense | sense | sense | |
| | GT | GT | 75% GT | 50% GT | 25% GT | 10% GT | 3% GT | p53 AT | p53 AT | |
| 3 | sense | sense | sense | sense | sense | sense | sense | sense | sense | sense |
| | AT | AT | 75% GT | 50% GT | 25% GT | 10% GT | 3% GT | GT | GT | AT |
| 4 | sense | sense | sense | sense | sense | sense | sense | AT | AT | sense |
| | | | 75% GT | 50% GT | 25% GT | 10% GT | 3% GT | | | AT |
| 5 | AT | AT | sense | sense | sense | sense | sense | sense | sense | sense |
| | | | p53 AT | p53 AT | AT | AT | GT | GT | | |
| 6 | | | p53 se | p53 se | p53 se | p53 se | p53 se | p53 se | p53 se | p53 se |
| | | | AT | AT | p53 AT | p53 AT | p53 GT | p53 GT | | |
| 7 | p53 AT | p53 AT | p53 se | p53 se | p53 se | p53 se | p53 se | p53 se | p53 se | p53 se |
| | | | 75% p53GT | 50% p53GT | 25% p53GT | 10% p53GT | 3% p53GT | | | p53 GT |
| 8 | p53 se | p53 se | p53 se | p53 se | p53 se | p53 se | p53 se | p53 se | p53 se | p53 se |
| | p53 GT | p53 GT | 75% p53GT | 50% p53GT | 25% p53GT | 10% p53GT | 3% p53GT | AT | AT | p53 GT |
| 9 | p53 se | p53 se | p53 se | p53 se | p53 se | p53 se | p53 se | p53 se | p53 AT | p53 AT |
| | p53 AT | p53 AT | 75% p53GT | 50% p53GT | 25% p53GT | 10% p53GT | 3% p53GT | p53 AT | | |
| 10 | | | p53 se | p53 se | p53 se | p53 se | p53 se | p53 se | p53 se | p53 se |
| | | | 75% p53GT | 50% p53GT | 25% p53GT | 10% p53GT | 3% p53GT | p53 AT | AT | AT |

TABLE 24
Measuring the red fluorescence intensity on an agarose chip for detecting the binding of
Cy ™ 5-labeled E. coli mutS to different mixtures of perfectly paired and
GT-mispaired DNA double strands. The table gives the positions on the chip together with the
appurtenant relative fluorescent intensities.

| Position | 1 | 2 | 3 | 4 | 5 | 6 | 7 | 8 | 9 | 10 |
|---|---|---|---|---|---|---|---|---|---|---|
| 1 | 24.057 | 23.473 | 159.973 | 135.717 | 103.890 | 75.523 | 40.092 | 8.811 | 8.751 | 9.479 |
| 2 | 211.207 | 198.842 | 173.694 | 142.125 | 102.809 | 67.751 | 42.987 | 21.791 | 19.666 | 9.538 |
| 3 | 46.586 | 71.004 | 182.958 | 149.908 | 101.801 | 70.750 | 43.959 | 195.088 | 215.093 | 44.053 |
| 4 | 18.916 | 19.874 | 193.065 | 171 .824 | 115.074 | 79.478 | 48.761 | 68.155 | 65.692 | 41.359 |
| 5 | 18.870 | 27.299 | 22.893 | 24.925 | 34.095 | 41.398 | 224.631 | 223.216 | 21.282 | 21.146 |
| 6 | 9.731 | 8.832 | 22.698 | 21.925 | 42.062 | 45.237 | 106.903 | 112.647 | 19.437 | 20.833 |
| 7 | 18.780 | 26.964 | 88.790 | 69.629 | 57.280 | 49.508 | 49.914 | 19.220 | 20.095 | 119.817 |
| 8 | 101.278 | 102.938 | 85.600 | 73.277 | 58.715 | 50.241 | 48.824 | 25.777 | 26.905 | 117.167 |
| 9 | 39.504 | 41.772 | 85.529 | 73.955 | 60.548 | 50.971 | 47.901 | 44.913 | 21.366 | 21.794 |
| 10 | 9.430 | 9.681 | 88.103 | 73.628 | 59.467 | 53.119 | 49.170 | 40.705 | 23.305 | 22.852 |

TABLE 25
Statistical analysis of the agarose chip containing different
mixtures of perfectly paired and GT-mispaired DNA double
strands. Mean values and standard deviations of the red
fluorescence intensities at all the positions having the same
loading were calculated in each case.

| First strand | Second strand | Result |
|---|---|---|
| sense | AT | 46.4 +/− 12.7 |
| sense | 3% GT | 43.9 +/− 3.6 |
| sense | 10% GT | 73.4 +/− 5.2 |
| sense | 25% GT | 105.9 +/− 6.2 |
| sense | 50% GT | 149.9 +/− 15.7 |
| sense | 75% GT | 177.4 +/− 14.1 |
| sense | 100% GT | 211.3 +/− 12.3 |
| P53 se | p53 AT | 42.4 +/− 2.3 |
| P53 se | 3% p53 GT | 49.0 +/− 0.8 |
| P53 se | 10% p53 GT | 51.0 +/− 1.6 |
| P53 se | 25% p53 GT | 59.0 +/− 1.4 |
| P53 se | 50% p53 GT | 72.6 +/− 2.0 |
| P53 se | 75% p53 GT | 87.0 +/− 1.7 |
| P53 se | 100% p53 GT | 110.1 +/− 7.6 |

Controls::

| | | |
|---|---|---|
| — | — | 9.3 +/− 0.4 |
| sense | — | 20.3 +/− 1.0 |
| P53 se | — | 19.9 +/− 0.6 |
| — | AT | 45.0 +/− 22.1 |
| — | P53 AT | 22.2 +/− 3.0 |
| sense | P53 AT | 22.3 +/− 1.9 |
| P53 se | AT | 24.7 +/− 1.7 |

FIG. 16 shows the binding of Cy™5-labeled E.coli mutS to different mixtures of perfectly paired and GT-mispaired DNA double strands which were produced by hybridizing on an electronically addressable agarose chip. The figure in each case depicts the mean red fluorescence intensity, together with standard deviation, for the different double strands. FIG. 16A depicts the binding of mutS to the double strands obtained using the “sense” first strand and the complementary AT (perfectly pairing) and GT (mispairing) oligonucleotides. FIG. 16B shows the binding of mutS to the double strands which are obtained using the p53 se oligonucleotide and the complementary p53 AT (perfectly pairing) and p53 GT (mispairing) counterstrands.

In the case of both the DNA sequences tested, it was possible to show, in a congruent manner, that mutS bound better even to a DNA mixture which contained 90% perfectly paired double strands, and only 10% GT-mispaired strands, than it did to a sample consisting of 100% perfectly paired DNA (Table 25; FIG. 16). In the case of the p53 se first-strand sequence, Cy™5 fluorescence which was measured was even higher than the value obtained with the 100% perfectly paired DNA when the proportion of mispaired DNA was only 3%. Accordingly, the method which is described here can be used to detect a mutation even when only a small proportion of the DNA strands to be tested contain the corresponding base substitution.

### Example: Detecting Mutations in Genomic DNA

In the following experiment, a check was carried out to determine whether it is possible to use mutS to detect mutations in a clinically relevant gene and whether it is possible to use the present invention to example PCR products of genes from patient samples for the presence of mutations.

The tumor suppressor gene p53 plays an important role in the genesis of cancer (B. Vogelstein, K. W. Kinzler, Cell 70 (1992), 523-526); accordingly, mutations in p53 can be used as a prognostic marker for the development of a tumor. More than 90% of all the known mutations in p53 are located in the region from Exon 5 to Exon 9 in the gene (M. Hollstein, D. Sidransky, B. Vogelstein, C. C Harris, Science 253, 49-53 (1991)), which region encodes the DNA-binding domain of the protein.

It was now checked to determine whether it is possible to use dye-labeled mutS to detect mutations in Exon 8 of the p53 gene in human cell lines on electronically addressable DNA microchips. For this, genomic DNA derived from the following 4 human tumor cell lines was obtain d from the Deutsche Sammlung für Mikroorganismen und Zellkulturen GmbH (DSMZ) (German Collection of Microorganisms and Cell Cultur s), Brunswick, Germany:

The numbering of the cell lines is in accordance with the labeling given by the DSMZ.

MCF-7 (DSMZ ACC 115) is an adenocarcinoma cell line which originated from mammary gland epithelium; no mutations are known to be present in p53 (Landers J E et al. Translational enhancement of mdm2 oncogene expression in human tumor cells containing a stabilized wild-type p53 protein. Cancer Res. 57: 3562-3568, 1997)

SW480 (DSMZ ACC 313): established from a human colorectal adenocarcinoma, contains a G to A mutation in codon 273 of Exon 8 in the p53 gene (Weiss J et al. Mutation and expression of the p53 gene in malignant melanoma cell lines. Int. J. Cancer 54: 693-699, 1993)

MOLT-4 (DSMZ ACC 362): human T-lymphoblast cell line, contains a G to A mutation in codon 248 of Exon 7 in p53 (Rodrigues N R et al. p53 mutations in colorectal cancer. Proc. Natl. Acad. Sci. USA 87: 7555-7559, 1990) 293 (DSMZ ACC 305) is an adenovirus-transformed human embryonic kidney epithelium cell line for which no mutations in p53 Exon 8 have been published.

Accordingly, only cell line SW-480, and possibly cell line 293, contains a mutation in Exon 8 of the p53 gene.

The polymerase chain reaction (PCR) was used to amplify Exon 8 from the genomic DNA of the above-described cell lines. The respective PCR products (length: 237 bp) were then hybridized on a hydrogel chip with a synthetic oligonucleotide (length: 73 bases) whose sequence corresponded to the wild-type sequence of the region being investigated. In order to prevent mutS from binding to the protruding, single-stranded ends of the PCR product, and consequently to prevent an increase in the nonspecific background fluorescence, the chip was treated with a single strand-specific endonuclease (mung bean nuclease) and a single strand-binding protein.

Binding of dye-labeled E.coli mutS to the different double strands were then investigated.

Implementation of the polymerase chain reaction:

| | | |
|---|---|---|
| Mixture per cell line: | 84.2 μl of | H₂O |
| | 10 μl of | 10x cloned Pfu DNA polymerase |
| | | reaction buffer (Stratagene, |
| | | Amsterdam, NL) |
| | 0.8 μl of | dNTP (in each case 25 mM) |
| | 2 ml of | genomic DNA (150 ng/μl) |
| | 0.5 μl of | Exon8for primer(Seq. ID No. 45), |
| | | 100 μM |
| | 0.5 μl of | Primer Exon8rev (Seq. ID No. 46), |
| | | 100 μM, Cy3-labeled |
| | 2 μl of | Pfu Turbo Hotstart DNA polymerase |
| | | (2.5 U/μl, Stratagene) |

The amplification was carried out in a Thermocycler (GeneAmp PCR System 2400, Perkin Elmer, Langen, Germany) under the following conditions:

initial denaturation: 95° C., 2 min

31 amplification cycles, in each case: 95° C., 30 sec−62° C., 30 sec−72° C., 1 min

concluding elongation: 72° C., 10 min

The PCR products were subsequently purified using the QIAquick PCR purification kit supplied by QIAGEN. While this purification took place in accordance with the manufacturer's instructions, an additional washing step with 75% ethanol was carried out before eluting the DNA. The DNA was finally eluted in 50 μl of water. An analysis of the DNA on a 1.8% agarose gel showed that approximately the same quantity of PCR product was obtained for all the cell lines.

Loading the Chip:

The biotinylated cExon8 (Seq. ID No. 47) oligonucloetide, which was used as the first strand, and the “sense”, “AT” and “GT” oligonucleotides which were used as positive and negative controls, were dissolved in 50 mM histidine buffer at a concentration of 100 nM. The purified PCR products which were used as second strands were in each case mixed with an equal volume of 100 nM histidine buffer. All the DNA strands were then denatured at 95° C. for 5 min. The cExon8 and “sense” first strands were electronically addressed to defined positions on a hydrogel chip for 60 sec, and at a voltage of 2.1 V, in the Nanogen workstation loading appliance. The hybridization with the Cy™3-labeled PCR products, or the “AT” and “GT” oligonucleotides, was carried out for 180 sec at 2.1 V. The loading scheme is depicted in Table 27.

After the loading, the chip was taken out of the loading appliance and filled with equilibration buffer; the green fluorescence at the chip positions was then measured in the Nanogen reader (instrument settings: medium gain, 256 ps integration time). The result of the measurement is given in Table 28. Subsequently, the chip was incubated at 30° C. for 45 min with 1 μl of mung bean nuclease (NEB, Frankfurt)+89 μl of 1× mung bean nuclease buffer (NEB). After the nuclease digestion, the chip was washed with 20 ml of equilibration buffer and the green fluorescence was measured once again. Table 29 shows the result of this measurement. The chip was then incubated at room temperature for 45 min with blocking buffer and subsequently for 30 min with a solution of 22 ng of SSB (single-stranded DNA binding protein, USB Corporation, Cleveland, USA)/μl in incubation buffer. Finally, the chip was incubated at room temperature for 60 min with 10 μl of Cy™5-labeled E. coli mutS (concentration: 50 ng/μl) in 90 μl of incubation buffer. After that, the chip was washed by hand with 1 ml of incubation buffer, inserted into the Nanogen reader and washed, in the reader and at a temperature of 37° C., 50× with in each case 0.25 ml of washing buffer. Finally, the Cy™5-fluorescence intensities at the individual positions on the chip were measured with the following instrument setting: high sensitivity (“high gain”); 256 μs integration time.

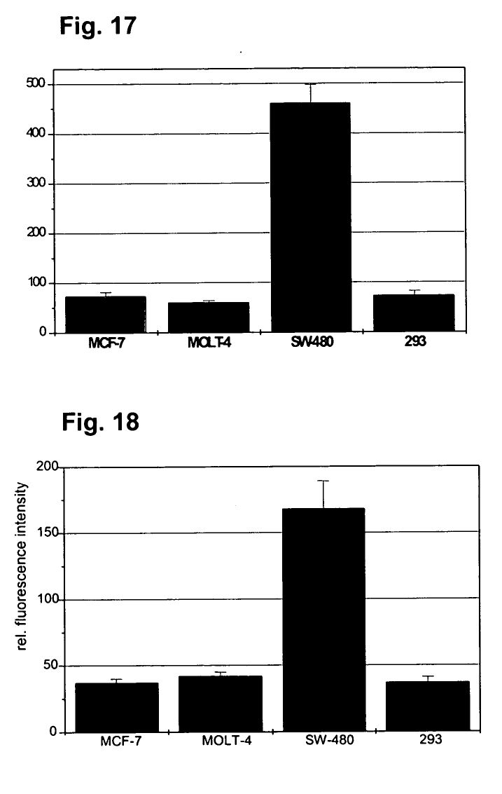

The red fluorescence intensities which were measured are given in Table 30; the results of the statistical analysis are depicted in Table 31 and in FIG. 17.

Buffers Employed:

50 mM histidine buffer: 50 mM L-histidine (Sigma); this solution was filtered through a 0.2 μm membrane and degassed by negative pressure:

100 mM histidine buffer: 100 mM L-histidine, filtered through a 0.2 μm membrane

Equilibration buffer: 20 mM tris, pH 7.6/50 mM KCl/5 mM MgCl₂/0.01% Tween-20

Blocking buffer: 20 mM tris, pH 7.6/50 mM KCl/5 mM MgCl₂/0.01% Tween-20/3% BSA

Incubation buffer: 20 mM tris, pH 7.6/50 mM KCl/5 mM MgCl₂/0.01% Tween-20/1% BSA

Washing buffer: 20 mM tris, pH 7.6/50 mM KCl/5 mM MgCl₂/0.1% Tween-20

TABLE 27
Scheme for loading a hydrogel chip for detecting mutations in Exon 8 of the p53 gene in human cell
lines. The individual positions were first of all addressed with the oligonucleotide named in the upper line and
subsequently hybridized with the PCR product of the different cell lines (MCF-7, MOLT-4, SW-480 and 293)
or with the AT or GT oligonucleotides, which PCR products or oligonucleotides are named in the second line.
Some positions were loaded only with first strand or second strand as controls: empty boxes symbolize positions
which were not loaded with DNA.

| Position | 1 | 2 | 3 | 4 | 5 | 6 | 7 | 8 | 9 | 10 |
|---|---|---|---|---|---|---|---|---|---|---|
| 1 | | | | | | | | | | |
| 2 | | cExon8 | cExon8 | cExon8 | cExon8 | cExon8 | cExon8 | cExon8 | MOLT-4 | MOLT-4 |
| | | MCF-7 | MCF-7 | 293 | 293 | SW-480 | SW-480 | MOLT-4 | | |
| 3 | | cExon8 | cExon8 | cExon8 | cExon8 | cExon8 | cExon8 | cExon8 | SW-480 | SW-480 |
| | | MOLT-4 | MOLT-4 | MCF-7 | MCF-7 | 293 | 293 | SW-480 | | |
| 4 | | cExon8 | cExon8 | cExon8 | cExon8 | cExon8 | cExon8 | cExon8 | 293 | 293 |
| | | SW-480 | SW-480 | MOLT-4 | MOLT-4 | MCF-7 | MCF-7 | 293 | | |
| 5 | | cExon8 | cExon8 | cExon8 | cExon8 | cExon8 | cExon8 | cExon8 | MCF-7 | MCF-7 |
| | | 293 | 293 | SW-480 | SW-480 | MOLT-4 | MOLT-4 | MCF-7 | | |
| 6 | | | | | | | | | | |
| 7 | | | | cExon8 | cExon8 | cExon8 | cExon8 | | | |
| 8 | | sense | sense | | | | | sense | sense | |
| | | AT | GT | | | | | AT | GT | |
| 9 | | sense | sense | | | | | sense | sense | |
| | | AT | GT | | | | | AT | GT | |
| 10 | | | | | | | | | | |

TABLE 28
Measurement of the green fluorescence intensity on the hydrogel chip before treating with mung bean
nuclease. The table gives the positions on the chip together with the appurtenant relative fluorescence
intensities.

| Position | 1 | 2 | 3 | 4 | 5 | 6 | 7 | 8 | 9 | 10 |
|---|---|---|---|---|---|---|---|---|---|---|
| 1 | 80.148 | 95.004 | 95.656 | 97.127 | 100.203 | 95.252 | 101.219 | 116.302 | 135.330 | 119.752 |
| 2 | 92.445 | >1049 | >1049 | >1049 | >1049 | >1049 | >1049 | 898.523 | 563.012 | 516.020 |
| 3 | 103.476 | 853.332 | 867.209 | >1049 | >1049 | >1049 | >1049 | >1049 | 410.191 | 436.986 |
| 4 | 92.029 | >1049 | >1049 | >1049 | >1049 | >1049 | >1049 | >1049 | 338.840 | 381.170 |
| 5 | 87.223 | >1049 | >1049 | >1049 | >1049 | >1049 | >1049 | >1049 | 378.905 | 408.152 |
| 6 | 76.940 | 113.215 | 132.941 | 122.102 | 112.650 | 134.214 | 139.220 | 147.301 | 152.500 | 127.936 |
| 7 | 80.792 | 138.522 | 168.927 | 10.960 | 9.651 | 9.781 | 10.359 | 162.171 | 185.828 | 113.988 |
| 8 | 107.746 | >1049 | >1049 | 221.678 | 104.610 | 101.467 | 159.058 | >1049 | >1049 | 180.904 |
| 9 | 104.911 | >1049 | >1049 | 199.967 | 106.491 | 101.637 | 173.489 | >1049 | >1049 | 177.673 |
| 10 | 65.581 | 105.148 | 142.067 | 99.861 | 89.024 | 85.426 | 99.001 | 131.723 | 128.933 | 94.787 |

TABLE 29
Measurement of the green fluorescence intensity on the hydrogel chip after treating with mung bean
nuclease. The table indicates the positions on the chip together with the appurtenant relative
fluorescence intensities.

| Position | 1 | 2 | 3 | 4 | 5 | 6 | 7 | 8 | 9 | 10 |
|---|---|---|---|---|---|---|---|---|---|---|
| 1 | 78.126 | 108.358 | 113.947 | 118.616 | 123.849 | 118.542 | 120.161 | 127.487 | 130.800 | 109.000 |
| 2 | 98.209 | 781.316 | 557.648 | 589.761 | 646.440 | 762.933 | 730.607 | 597.336 | 515.812 | 482.538 |
| 3 | 112.364 | 550.508 | 591.713 | 512.339 | 567.977 | 552.579 | 505.046 | 733.454 | 338.130 | 369.904 |
| 4 | 105.444 | 809.800 | 699.284 | 714.060 | 685.824 | 486.339 | 520.774 | 571.883 | 280.361 | 331.984 |
| 5 | 97.862 | 808.656 | 761.990 | 673.812 | 741.593 | 776.637 | 754.193 | 598.984 | 312.768 | 345.928 |
| 6 | 76.175 | 132.207 | 146.339 | 137.655 | 124.691 | 140.119 | 146.962 | 151.590 | 143.940 | 115.469 |
| 7 | 80.445 | 142.893 | 174.909 | 9.916 | 9.032 | 9.225 | 9.375 | 164.812 | 180.101 | 106.676 |
| 8 | 108.136 | >1049 | >1049 | 217.390 | 100.034 | 98.901 | 158.589 | >1049 | >1049 | 170.876 |
| 9 | 103.864 | >1049 | >1049 | 193.048 | 99.668 | 98.269 | 166.062 | >1049 | >1049 | 161.325 |
| 10 | 62.256 | 103.774 | 135.682 | 98.012 | 83.341 | 80.891 | 95.245 | 124.523 | 127.216 | 83.755 |

TABLE 30
Measuring the red fluorescence intensity for detecting the binding of Cy ™ 5-labeled E. coli mutS to
double strands consisting of a synthetic oligonucleotide and PCR products from different cell lines.
The table gives the positions on the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 9.533 | 11.773 | 12.402 | 12.451 | 12.323 | 14.383 | 12.336 | 13.558 | 10.751 | 9.535 |
| 2 | 9.964 | 70.465 | 64.235 | 67.810 | 54.356 | 444.449 | 422.986 | 59.040 | 41.433 | 42.802 |
| 3 | 10.889 | 56.552 | 53.921 | 68.300 | 60.152 | 71.028 | 76.700 | 470.869 | 48.533 | 47.720 |
| 4 | 10.858 | 521.583 | 417.066 | 60.643 | 55.186 | 81.802 | 82.523 | 85.367 | 40.069 | 43.933 |
| 5 | 10.153 | 83.704 | 70.291 | 447.909 | 501.967 | 66.511 | 64.786 | 81.755 | 47.413 | 45.402 |
| 6 | 10.297 | 11.915 | 14.569 | 13.015 | 11.843 | 11.523 | 12.473 | 13.173 | 11.874 | 9.510 |
| 7 | 10.400 | 17.060 | 22.810 | 87.783 | 54.149 | 39.615 | 61.436 | 19.748 | 23.324 | 10.934 |
| 8 | 11.212 | 150.350 | >1049 | 17.007 | 10.171 | 11.107 | 14.666 | 154.423 | >1049 | 15.465 |
| 9 | 10.252 | 150.067 | >1049 | 14.882 | 10.106 | 10.377 | 13.522 | 171.733 | >1049 | 13.987 |
| 10 | 8.700 | 10.496 | 12.980 | 9.971 | 9.567 | 9.916 | 10.289 | 11.816 | 12.917 | 24.527 |

TABLE 31
Statistical analysis of the results from the hydrogel chip used
for detecting mutations in Exon 8 of the p53 gene in various cell
lines. The mean values and standard deviations of the red
fluorescence intensities at all the positions having the same loading
were calculated in each case.

| | | Mean value +/− standard |
|---|---|---|
| First strand | Second strand | deviation |
| cExon8 | — | 60.7 +/− 17.5 |
| cExon8 | PCR product MCF-7 | 72.8 +/− 8.6 |
| cExon8 | PCR product MOLT-4 | 59.5 +/− 4.4 |
| cExon8 | PCR product SW-480 | 461.0 +/− 36.4 |
| cExon8 | PCR product 293 | 72.7 +/− 9.8 |
| — | PCR product MCF-7 | 46.4 +/− 1.0 |
| — | PCR product MOLT-4 | 42.1 +/− 0.7 |
| — | PCR product SW-480 | 48.1 +/− 0.4 |
| — | PCR product 293 | 42.0 +/− 1.9 |
| Sense | AT | 156.6 +/− 8.7 |
| Sense | GT | >1049 |

FIG. 17 shows the binding of Cy™5-labeled E.coli mutS to double strands, consisting of a synthetic oligonucleotide and PCR products from different cell lines, for detecting mutations in Exon 8 of the p53 gene. In each case, the figure depicts the mean red fluorescence intensity, together with standard deviation, for the individual cell lines.

As is evident from Table 28, all the positions hybridized with the different PCR fragments exhibited a green fluorescence of similar magnitude; consequently, approximately the same quantity of PCR product was bound at each of these positions. The green fluorescence intensities were markedly less after the nuclease digestion (Table 29) than before the digestion. This suggests that the degradation of single-stranded DNA regions on the chip worked well.

When analyzing the red fluorescence (Table 31, FIG. 17), it was found that the E.coli mutS bound preferentially to those positions on the chip at which the cExon8 oligonucleotide had been hybridized with the PCR product from the SW-480 cell line: in these cases, the fluorescence intensities were about 6.5 times higher than at the positions at which hybridization with the PCR products of the cell lines MCF-7, MOLT-4 or 293 had taken place. The method described here was consequently successful in detecting the base substitution mutation, which is known to be present in cell line SW480, in codon 273 of the p53 gene. Under the chosen experimental conditions, this base substitution led to a GT mispairing which was readily recognized by the dye-labeled mutS. By contrast, very similar fluorescence intensities were measured in the case of cell line 293, for which there is no information regarding any possible mutations in p53, as were measured in the case of line MCF-7, which does not contain any mutations in the p53 gene. This suggests that cell line 293 does not contain any base substitution in the investigated region, either.

In summary, it was possible to demonstrate, by means of this experiment, that the method which is described here is well suited for detecting mutations in DNA isolated from patient samples. Consequently, a DNA chip-based system which is suitable for the parallelized, high-throughput detection of mutations has been published for the first time in the present invention.

### Example: Alternative Method for Detecting Mutation in Genomic DNA

An examination was subsequently carried out to determine whether the previously described method for the mutS-mediated detection of mutations in genomic DNA also works when (“capturing agent”) biotinylated PCR products are used as the first strand in place of synthetic oligonucleotides. The use of PCR products as “capturing agents” would make it possible to examine longer DNA fragments for the presence of mutations.

However, in this connection, the fact has to be taken into consideration that, in contrast to synthetic oligonucleotides, PCR products are initially present as double strands. If such a PCR product were to be addressed to a microchip without any further purification, the complementary counterstrand would then immediately attack the biotinylated “capturing agent” strand and thereby obstruct the subsequent hybridization with the (“target”) DNA to be tested. In order to avoid this problem, the biotinylated strand which was used at the first strand was firstly separated from the complementary counterstrand.

In order to be able to compare the two methods for detecting mutations, different positions on electronically addressable hydrogel chips were first of all addressed with the single-stranded, biotinylated PCR product from the wild-type cell line MCF-7 or with the synthetic oligonucleotide cExon8, as the first strand, and subsequently hybridized with the PCR products from different cell lines as the “targets”.

In parallel with this, it was also desired to test more accurately whether the treatment of the chips with single strand-specific endonuclease and SSB (single strand-binding protein) is advantageous for recognizing mutations in genomic DNA or whether these incubation steps can be omitted without any loss of sensitivity. In order to investigate this, four hydrogel chips were loaded with first and second strands in accordance with the same scheme and subsequently treated in accordance with different incubation protocols.

The single-stranded, biotinylated PCR products were prepared in accordance with the following scheme: genomic DNA from the cell line MCF-7, which does not contain any mutation in the p53 gene, was used as the starting material for the PCR.

| | | |
|---|---|---|
| PCR mixture: | 84.2 μl of | H₂O |
| | 10 μl of | 10x cloned Pfu DNA polymerase reaction |
| | | buffer (Stratagene, Amsterdam, NL) |
| | 0.8 μl of | dNTP (in each case 25 mM) |
| | 2 μl of | genomic DNA from the cell line MCF-7 |
| | | (150 ng/μl) |
| | 0.5 μl of | Exon8for_bio primer (Seq. ID No. 48), |
| | | 100 μM, biotinylated |
| | 0.5 μl of | Exon8rev_b primer (Seq. ID No. 49), |
| | | 100 μM |
| | 2 μl of | Pfu Turbo Hotstart DNA polymerase |
| | | (2.5 U/μl, Stratagene) |

The amplification took place in a thermocycler (GeneAmp PCR System 2400, Perkin Elmer, Langen, Germany) under the following conditions:

initial denaturation (95° C., 2 min), followed by 31 amplification cycles (in each case 95° C., 30 sec−62° C., 30 sec−72° C., 1 min) and concluding elongation (72° C., 10 min)

In order to separate off excess biotinylated primers, the PCR products were then purified using the QIAquick PCR purification kit supplied by QIAGEN. While this purification took place in accordance with the manufacturer's instructions, an additional washing step with 75% ethanol was carried out prior to eluting the DNA. The DNA was finally eluted in 50 μl of water. The biotinylated single strands were isolated using magnetic, streptavidin-coated beads supplied by DYNAL Biotech (Hamburg). For this, the PCR product was diluted with an equal volume of 2×B&W buffer (10 mM tris-HCl, pH 7.5, 1 mM EDTA, 2 M NaCl), with this solution then being mixed with Dynabeads M-280 streptavidin and incubated at room temperature for 15 min, while shaking carefully, in order to enable the biotinylated DNA strands to bind to the streptavidin. The beads were then concentrated in a magnet (DYNAL MPC-S) and the supernatant was discarded; the beads were then washed with 1×B&W buffer. In order to separate off the non-biotinylated counterstrands, the beads were then suspended in 0.1 M NaOH and incubated at room temperature for 5 min; after that, they were washed, in each case once, with 0.1 M NaOH, with 1×B&W buffer and with water. In order to release the biotinylated single strands from the streptavidin, the beads were finally suspended in 95% formamide/10 mM EDTA, pH 8.0, and incubated at 65° C. for 4 min. The supematant, containing the biotinylated single strands, was taken off while still hot and subsequently purified using the QIAquick PCR purification kit.

The “targets” were prepared as described in the example entitled “Detecting mutations in Genomic DNA” under “Implementation of the polymerase chain reaction”.

Loading the hydrogel chip: the single-stranded, biotinylated PCR product (“ssPCR”) was diluted with an equal volume of 100 mM histidine buffer. The biotinylated cExon8 oligonucleotide (Seq. ID No. 47), and also the “APC se” (Seq. ID No. 24), “APC AT” (Seq. ID No. 25) and “APC GT” (Seq. ID No. 26) oligonucleotides, which were used as positive and negative controls, were dissolved in 50 mM histidine buffer at a concentration of 100 mM. The purified PCR products which were used as second strands were in each case mixed with an equal volume of 100 mM histidine buffer. All the DNA strands were then denatured at 95° C. for 5 min. The electronic addressing of the different first strands to defined positions on the hydrogel chip was effected, for 60 sec and at a voltage of 2.1 V, in the Nanogen workstation loading appliance. The hybridization with the Cy™3-labeled PCR products and the “APC AT” and “APC GT” oligonucleotides was carried out for 180 sec at 2.1 V. The loading scheme, which was identical for all 4 chips, is depicted in Table 32.

Buffers Employed:

50 mM histidine buffer: 50 mM L-histidine, filtered through a 0.2 μm membrane and degassed

100 mM histidine buffer: 100 mM L-histidine, filtered through a 0.2 μm membrane

Further Treatment of the Chips:

After having been loaded, two of the hydrogel chips (subsequently termed chips A and B) were incubated at room temperature for 70 min with blocking buffer. Chip B was then additionally incubated for 45 min with 22 ng/μl SSB (single-stranded DNA binding protein, USB Corporation, Cleveland, USA) in incubation buffer. Finally, both chips were in each case incubated, at room temperature for 60 min., with 3 μl of Cy™5-labeled E.coli-MBP mutS (concentration: 450 ng/μl) in 97 μl of incubation buffer. After that, the chips were washed by hand with 1 ml of incubation buffer, inserted into the Nanogen reader and washed, in the reader and at a temperature of 37° C., 50× with in each case 0.25 ml of washing buffer. Finally, the fluorescence intensities at the individual positions on the chips were measured (instrument setting: “high gain”, 256 μs integration time for red fluorescence; “medium gain”, 256 μs for green fluorescence).

After having been loaded, chips C and D were filled with equilibration buffer and the green fluorescence at the positions on the chips was measured in the Nanogen reader (“medium gain”, 256 μs integration time). Subsequently, the chips were incubated, at 30° C. for 45 min, with 1 μl of mung bean nuclease (NEB, Frankfurt)+89 μl of 1× mung bean nuclease buffer (NEB). After the nuclease digestion, the chips were washed with 20 ml of equilibration buffer and then incubated with blocking buffer at room temperature for 70 min. After that, chip D was additionally incubated for 45 min with 22 ng/μl SSB (single-stranded DNA binding protein) in incubation buffer. Finally, both chips were in each case incubated, at room temperature for 6 min, with 3 μl of Cy™5-labeled E. coli-MBP mutS (concentration: 450 ng/μl) in 97 μl of incubation buffer. After that, the chips were in each case washed with 1 ml of incubation buffer and then washed in the Nanogen reader, at a temperature of 37° C., 50× with in each case 0.25 ml of washing buffer. Finally, the fluorescence intensities at the individual positions on the chips were measured (red fluorescence: “high gain”, 256 μs; green fluorescence: “medium gain”, 256 μs)

Buffers Employed:

Equilibration buffer: 20 mM tris, pH 7.6/50 mM KCl/5 mM MgCl₂/0.01% Tween-20

Blocking buffer: 20 mM tris, pH 7.6/50 mM KCl/5 mM MgCl₂/0.01% Tween-20/3% BSA

Incubation buffer: 20 mM tris, pH 7.6/50 mM KCl/5 mM MgCl₂/0.01% Tween-20/1% BSA

Washing buffer: 20 mM tris, pH 7.6/50 mM KCl/5 mM MgCl₂/0.1% Tween-20

The green and red fluorescence values which were measured in the case of chip A (without nuclease and without SSB) are listed in Tables 33 and 34, while the values in the case of chip B (without nuclease but with SSB) are listed in Tables 35 and 36.

The results of the green fluorescence measurement carried out on chip C (with nuclease but without SSB) prior to the nuclease digestion are listed in Table 37, while the green and red fluorescence values following incubation with mutS are to be found in Tables 38 and 39. The green fluorescences obtained for chip D (with nuclease and with SSB) prior to the nuclease treatment are given in Table 40 and the results obtained from measuring the green and red fluorescence after incubation with mutS are given in Tables 41 and 42.

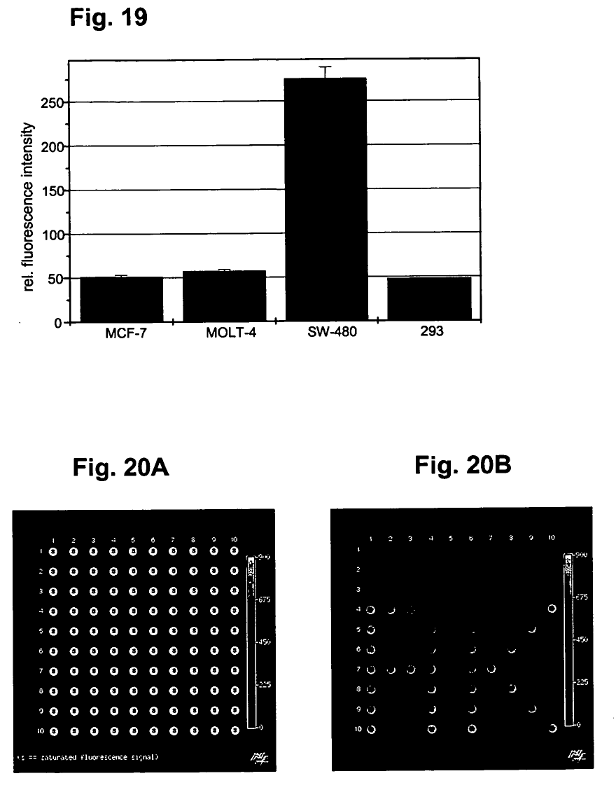

The results of the statistical analysis of all four chips are summarized in Table 43. FIGS. 18 and 19 additionally illustrate the results obtained with chip D in the form of histograms.

TABLE 32
Scheme for loading 4 hydrogel chips for comparing different methods for detecting mutations in Exon
8 of the p53 gene. The individual positions on the hydrogel chips were firstly addressed with the
cExon8 or APC se oligonucleotides, or with the biotinylated, single-stranded PCR product (“ssPCR”),
which are named in the upper line. Subsequently, hybridization was carried out with the PCR products
from the different cell lines (MCF-7, MOLT-4, SW-480 and 293), or with the APC AT or APC GT
oligonucleotides, which are named in the second line. Some positions were only loaded with first or
second strands as controls. Empty boxes symbolize positions which were not loaded with DNA.

| | | | | | | | | | | |
|---|---|---|---|---|---|---|---|---|---|---|
| Position | 1 | 2 | 3 | 4 | 5 | 6 | 7 | 8 | 9 | 10 |

| | | | | | | | | |
|---|---|---|---|---|---|---|---|---|
| 1 | | | | | | | | |
| 2 | cExon8 | cExon8 | cExon8 | cExon8 | cExon8 | cExon8 | MOLT-4 | MOLT-4 |
| | MCF-7 | MCF-7 | 293 | 293 | SW-480 | SW-480 | | |
| 3 | cExon8 | cExon8 | cExon8 | cExon8 | cExon8 | cExon8 | SW-480 | SW-480 |
| | MOLT-4 | MOLT-4 | MCF-7 | MCF-7 | 293 | 293 | | |
| 4 | cExon8 | cExon8 | cExon8 | cExon8 | cExon8 | cExon8 | 293 | 293 |
| | SW-480 | SW-480 | MOLT-4 | MOLT-4 | MCF-7 | MCF-7 | | |
| 5 | cExon8 | cExon8 | cExon8 | cExon8 | cExon8 | cExon8 | MCF-7 | MCF-7 |
| | 293 | 293 | SW-480 | SW-480 | MOLT-4 | MOLT-4 | | |
| 6 | | ssPCR | ssPCR | ssPCR | ssPCR | ssPCR | ssPCR | ssPCR |
| | | MCF-7 | MOLT-4 | MOLT-4 | SW-480 | SW-480 | 293 | 293 |
| 7 | | | ssPCR | cExon8 | cExon8 | cExon8 | | |
| | | | MCF-7 | | | | | |
| 8 | APC se | APC se | | | | | APC se | APC se |
| | APC AT | APC GT | | | | | APC AT | APC GT |
| 9 | APC se | APC se | | ssPCR | ssPCR | | APC se | APC se |
| | APC AT | APC GT | | | | | APC AT | APC GT |
| 10 | | | | | | | | |

TABLE 33
Measuring the green fluorescence of chip A (without nuclease and without SSB). The table gives the
positions on the chip together with the appurtenant relative fluorescence intensities following incubation with
mutS.

| Position | 1 | 2 | 3 | 4 | 5 | 6 | 7 | 8 | 9 | 10 |
|---|---|---|---|---|---|---|---|---|---|---|
| 1 | 4.954 | 4.937 | 5.039 | 5.130 | 5.061 | 5.076 | 5.088 | 5.259 | 5.181 | 5.131 |
| 2 | 4.950 | >1049 | 822.033 | >1049 | >1049 | >1049 | >1049 | 139.184 | 135.993 | 5.186 |
| 3 | 4.894 | 374.225 | 535.417 | 857.394 | 851.899 | 766.655 | >1049 | 159.327 | 163.433 | 5.195 |
| 4 | 4.960 | 956.508 | 754.370 | 470.287 | 522.184 | >1049 | >1049 | 61.346 | 69.399 | 5.196 |
| 5 | 5.030 | >1049 | 922.343 | 725.166 | 666.783 | 562.442 | 626.411 | 95.532 | 112.843 | 5.258 |
| 6 | 4.976 | 4.875 | 854.966 | 649.521 | 644.856 | 789.313 | 880.920 | 903.024 | >1049 | 5.277 |
| 7 | 5.168 | 5.850 | 6.189 | 837.161 | 6.492 | 6.097 | 6.224 | 6.822 | 6.555 | 5.326 |
| 8 | 5.779 | >1049 | >1049 | 5.435 | 5.138 | 5.162 | 5.537 | >1049 | >1049 | 5.877 |
| 9 | 5.574 | >1049 | >1049 | 6.008 | 6.346 | 6.299 | 5.431 | >1049 | >1049 | 6.399 |
| 10 | 5.601 | 5.938 | 5.697 | 5.266 | 4.993 | 4.993 | 5.123 | 5.976 | 6.770 | 5.791 |

TABLE 34
Measuring the red fluorescence of chip A (without nuclease and without SSB) for detecting the
binding of Cy ™ 5-labeled E. coli MBP mutS. The table gives the positions on the
chip together with the appurtenant relative fluorescent intensities.

| Position | 1 | 2 | 3 | 4 | 5 | 6 | 7 | 8 | 9 | 10 |
|---|---|---|---|---|---|---|---|---|---|---|
| 1 | 31.828 | 33.552 | 32.311 | 29.472 | 31.527 | 32.732 | 32.569 | 34.892 | 26.375 | 35.077 |
| 2 | 30.831 | 510.305 | 554.604 | 531.359 | 488.991 | 628.868 | 630.332 | 353.698 | 350.307 | 34.177 |
| 3 | 33.287 | 511.735 | 513.410 | 439.806 | 420.931 | 383.606 | 427.742 | 325.848 | 368.881 | 33.221 |
| 4 | 30.843 | 527.142 | 504.641 | 476.351 | 471.609 | 474.562 | 410.226 | 184.330 | 214.599 | 34.202 |
| 5 | 30.384 | 469.148 | 442.752 | 563.466 | 496.319 | 495.128 | 526.270 | 316.675 | 341.953 | 37.277 |
| 6 | 30.210 | 25.038 | 507.134 | 377.775 | 345.078 | 380.088 | 436.243 | 389.295 | 398.185 | 40.533 |
| 7 | 28.873 | 29.306 | 42.846 | 439.995 | 490.520 | 487.755 | 535.488 | 47.572 | 43.771 | 36.925 |
| 8 | 28.953 | 137.887 | >1049 | 31.766 | 42.103 | 43.174 | 34.293 | 160.870 | >1049 | 41.073 |
| 9 | 29.883 | 124.844 | >1049 | 35.145 | 850.664 | 858.499 | 35.607 | 140.665 | >1049 | 43.592 |
| 10 | 28.765 | 27.438 | 26.812 | 28.617 | 33.209 | 35.430 | 31.712 | 31.896 | 40.965 | 39.870 |

TABLE 35
Measuring the green fluorescence of chip B (without nuclease but with SSB). The table gives the
positions on the chip together with the appurtenant relative fluorescence intensities following incubation
with mutS.

| Position | 1 | 2 | 3 | 4 | 5 | 6 | 7 | 8 | 9 | 10 |
|---|---|---|---|---|---|---|---|---|---|---|
| 1 | 4.865 | 5.040 | 5.110 | 5.222 | 5.236 | 5.172 | 5.307 | 5.270 | 5.171 | 5.029 |
| 2 | 4.980 | >1049 | >1049 | >1049 | >1049 | >1049 | >1049 | 166.868 | 155.011 | 5.075 |
| 3 | 4.952 | 503.652 | 580.245 | >1049 | >1049 | >1049 | >1049 | 177.781 | 191.148 | 5.023 |
| 4 | 4.999 | >1049 | 865.819 | 532.746 | 578.989 | >1049 | >1049 | 114.693 | 80.921 | 5.025 |
| 5 | 5.021 | >1049 | >1049 | 830.953 | 791.859 | 541.138 | 705.363 | 118.087 | 142.166 | 5.090 |
| 6 | 5.009 | 4.944 | 895.117 | 722.565 | 686.743 | 845.736 | >1049 | >1049 | >1049 | 5.094 |
| 7 | 5.163 | 6.479 | 7.997 | 925.618 | 7.551 | 6.519 | 6.684 | 9.254 | 8.724 | 5.479 |
| 8 | 6.095 | >1049 | >1049 | 6.350 | 5.366 | 5.317 | 6.040 | >1049 | >1049 | 7.496 |
| 9 | 6.592 | >1049 | >1049 | 5.943 | 6.723 | 6.943 | 6.120 | >1049 | >1049 | 10.162 |
| 10 | 5.791 | 7.087 | 6.430 | 5.377 | 5.376 | 5.466 | 5.693 | 7.332 | 8.020 | 6.494 |

TABLE 36
Measuring the red fluorescence of chip B (without nuclease but with SSB) for detecting
the binding of Cy ™ 5-labeled E. coli MBP mutS. The table gives the positions on
the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 15.160 | 16.510 | 17.207 | 16.947 | 16.551 | 17.572 | 17.650 | 17.509 | 15.708 | 16.137 |
| 2 | 16.544 | 221.718 | 184.793 | 163.260 | 165.199 | 238.243 | 253.706 | 61.472 | 58.554 | 17.814 |
| 3 | 18.420 | 161.415 | 153.238 | 208.422 | 192.120 | 181.286 | 181.562 | 62.787 | 71.573 | 16.924 |
| 4 | 17.276 | 269.585 | 244.370 | 176.810 | 171.348 | 200.102 | 177.424 | 40.766 | 42.586 | 18.089 |
| 5 | 17.375 | 230.391 | 223.635 | 273.796 | 250.809 | 169.415 | 212.194 | 62.143 | 64.137 | 17.459 |
| 6 | 18.166 | 17.658 | 111.160 | 110.923 | 124.371 | 279.153 | 290.219 | 68.217 | 66.274 | 19.227 |
| 7 | 17.438 | 17.805 | 21.385 | 135.158 | 361.046 | 331.805 | 310.802 | 21.093 | 17.716 | 20.007 |
| 8 | 17.748 | 78.567 | >1049 | 19.746 | 20.118 | 18.952 | 18.868 | 77.927 | >1049 | 26.695 |
| 9 | 19.673 | 85.780 | >1049 | 21.120 | 327.748 | 325.396 | 19.657 | 79.054 | >1049 | 32.944 |
| 10 | 17.686 | 22.735 | 21.836 | 20.013 | 19.726 | 19.397 | 19.827 | 21.939 | 25.376 | 22.368 |

TABLE 37
Measuring the relative green fluorescence intensities of chip C prior to nuclease digestion.

| Position | 1 | 2 | 3 | 4 | 5 | 6 | 7 | 8 | 9 | 10 |
|---|---|---|---|---|---|---|---|---|---|---|
| 1 | 6.191 | 6.320 | 6.408 | 6.613 | 6.627 | 6.739 | 6.824 | 7.107 | 7.144 | 7.178 |
| 2 | 6.130 | >1049 | >1049 | >1049 | 933.757 | >1049 | >1049 | 238.918 | 248.649 | 7.176 |
| 3 | 6.121 | 891.877 | 929.986 | >1049 | >1049 | >1049 | >1049 | 182.051 | 275.933 | 6.890 |
| 4 | 6.236 | >1049 | >1049 | 902.917 | 898.767 | >1049 | >1049 | 92.266 | 115.634 | 7.093 |
| 5 | 6.176 | >1049 | >1049 | >1049 | >1049 | >1049 | >1049 | 123.853 | 143.181 | 6.992 |
| 6 | 6.153 | 5.830 | >1049 | 864.041 | 925.001 | >1049 | >1049 | >1049 | >1049 | 6.964 |
| 7 | 6.109 | 6.814 | 7.953 | >1049 | 8.459 | 7.906 | 7.794 | 8.090 | 8.107 | 6.955 |
| 8 | 6.645 | >1049 | >1049 | 6.809 | 6.424 | 6.424 | 6.657 | >1049 | >1049 | 7.618 |
| 9 | 6.387 | >1049 | >1049 | 6.888 | 8.146 | 8.256 | 6.676 | >1049 | >1049 | 9.052 |
| 10 | 6.209 | 7.100 | 7.181 | 6.310 | 6.272 | 6.343 | 6.468 | 8.060 | 8.860 | 7.176 |

TABLE 38
Measuring the relative green fluorescence intensities of chip C (with nuclease but without SSB)
following incubation with mutS

| Position | 1 | 2 | 3 | 4 | 5 | 6 | 7 | 8 | 9 | 10 |
|---|---|---|---|---|---|---|---|---|---|---|
| 1 | 4.965 | 5.034 | 5.074 | 5.102 | 5.073 | 5.116 | 5.140 | 5.133 | 5.102 | 5.085 |
| 2 | 5.015 | 96.705 | 86.405 | 90.794 | 70.036 | 130.784 | 187.055 | 155.578 | 158.099 | 5.105 |
| 3 | 5.035 | 174.678 | 161.404 | 79.320 | 68.297 | 91.579 | 104.372 | 101.787 | 164.901 | 5.126 |
| 4 | 5.035 | 203.143 | 152.118 | 129.972 | 126.397 | 96.542 | 104.906 | 37.175 | 44.905 | 5.136 |
| 5 | 5.039 | 152.436 | 113.756 | 136.756 | 131.906 | 162.734 | 189.183 | 55.578 | 66.398 | 5.149 |
| 6 | 5.024 | 4.548 | 560.419 | 478.025 | 477.072 | 623.471 | 629.020 | 701.968 | 745.073 | 5.165 |
| 7 | 5.000 | 5.332 | 5.501 | 681.204 | 6.799 | 6.383 | 6.343 | 5.441 | 5.391 | 5.106 |
| 8 | 5.074 | >1049 | >1049 | 5.214 | 5.139 | 5.192 | 5.171 | >1049 | >1049 | 5.182 |
| 9 | 5.034 | >1049 | >1049 | 5.170 | 6.325 | 6.316 | 5.221 | >1049 | >1049 | 5.154 |
| 10 | 5.063 | 5.092 | 5.151 | 5.093 | 5.084 | 5.106 | 5.123 | 5.212 | 5.172 | 4.998 |

TABLE 39
Measuring the red fluorescence of chip C (with nuclease but without SSB) for detecting
the binding of Cy ™ 5-labeled E. coli MBP mutS. The table gives the positions on
the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 16.093 | 15.349 | 16.371 | 15.943 | 16.512 | 15.900 | 16.460 | 16.973 | 16.458 | 13.973 |
| 2 | 16.729 | 46.306 | 37.055 | 34.375 | 29.887 | 156.988 | 141.521 | 42.443 | 43.947 | 16.279 |
| 3 | 16.348 | 45.717 | 43.093 | 35.619 | 31.523 | 35.624 | 34.530 | 34.627 | 39.001 | 16.464 |
| 4 | 16.617 | 174.811 | 153.331 | 40.797 | 36.146 | 36.601 | 37.179 | 26.443 | 31.046 | 16.363 |
| 5 | 16.792 | 49.694 | 49.633 | 153.700 | 128.807 | 39.757 | 46.605 | 50.119 | 40.810 | 16.612 |
| 6 | 16.929 | 16.281 | 55.556 | 57.775 | 52.015 | 263.902 | 254.834 | 53.350 | 56.445 | 16.620 |
| 7 | 16.157 | 16.662 | 20.123 | 56.064 | 15.537 | 17.747 | 17.949 | 17.804 | 17.629 | 17.177 |
| 8 | 16.278 | 55.325 | >1049 | 17.226 | 15.675 | 17.144 | 17.367 | 51.164 | >1049 | 17.412 |
| 9 | 16.156 | 51.671 | >1049 | 16.830 | 17.013 | 17.020 | 16.907 | 50.799 | >1049 | 17.589 |
| 10 | 16.770 | 16.890 | 16.012 | 16.846 | 16.516 | 16.622 | 16.637 | 16.325 | 17.826 | 17.450 |

TABLE 40
Measuring the relative green fluorescence intensities of chip D prior to nuclease digestion.

| Position | 1 | 2 | 3 | 4 | 5 | 6 | 7 | 8 | 9 | 10 |
|---|---|---|---|---|---|---|---|---|---|---|
| 1 | 8.984 | 9.126 | 9.118 | 9.323 | 9.235 | 8.556 | 9.506 | 10.063 | 9.870 | 9.943 |
| 2 | 8.791 | >1049 | >1049 | >1049 | >1049 | >1049 | >1049 | 268.093 | 277.511 | 9.664 |
| 3 | 8.799 | >1049 | >1049 | >1049 | >1049 | >1049 | >1049 | 203.322 | 305.893 | 9.048 |
| 4 | 8.738 | >1049 | >1049 | >1049 | >1049 | >1049 | >1049 | 101.569 | 84.196 | 9.105 |
| 5 | 8.514 | >1049 | >1049 | >1049 | >1049 | >1049 | >1049 | 139.880 | 137.902 | 8.961 |
| 6 | 8.418 | 7.772 | >1049 | 912.469 | >1049 | >1049 | >1049 | >1049 | >1049 | 8.874 |
| 7 | 8.411 | 10.004 | 13.146 | >1049 | 9.994 | 9.161 | 9.245 | 12.806 | 11.801 | 9.058 |
| 8 | 8.783 | >1049 | >1049 | 9.837 | 8.526 | 8.606 | 9.043 | >1049 | >1049 | 9.804 |
| 9 | 8.614 | >1049 | >1049 | 9.401 | 9.958 | 10.261 | 8.813 | >1049 | >1049 | 11.376 |
| 10 | 8.187 | 9.711 | 10.658 | 8.349 | 8.283 | 8.299 | 8.464 | 10.331 | 10.938 | 10.023 |

TABLE 41
Measuring the relative green fluorescence intensities of chip D (with nuclease and with SSB)
following incubation with mutS.

| Position | 1 | 2 | 3 | 4 | 5 | 6 | 7 | 8 | 9 | 10 |
|---|---|---|---|---|---|---|---|---|---|---|
| 1 | 4.829 | 4.974 | 4.993 | 4.983 | 4.980 | 5.191 | 5.027 | 5.076 | 5.007 | 5.016 |
| 2 | 4.947 | 104.583 | 102.576 | 103.513 | 105.658 | 169.028 | 169.631 | 154.565 | 166.585 | 5.009 |
| 3 | 4.938 | 157.383 | 149.468 | 89.924 | 92.150 | 88.634 | 100.377 | 103.543 | 168.834 | 4.958 |
| 4 | 5.004 | 128.439 | 148.039 | 130.810 | 119.248 | 91.479 | 108.615 | 35.775 | 28.826 | 4.983 |
| 5 | 5.021 | 116.366 | 108.364 | 108.475 | 126.060 | 137.974 | 113.599 | 48.976 | 46.977 | 5.020 |
| 6 | 4.950 | 4.608 | 678.399 | 421.217 | 530.679 | 651.647 | 792.985 | >1049 | >1049 | 5.019 |
| 7 | 4.978 | 5.217 | 5.428 | 813.096 | 6.699 | 6.125 | 6.308 | 5.517 | 5.364 | 5.018 |
| 8 | 4.945 | >1049 | >1049 | 5.198 | 5.070 | 5.105 | 5.092 | >1049 | >1049 | 5.135 |
| 9 | 5.038 | >1049 | >1049 | 5.110 | 6.024 | 6.029 | 5.159 | >1049 | >1049 | 5.068 |
| 10 | 4.953 | 5.114 | 6.024 | 5.064 | 4.964 | 5.002 | 5.103 | 5.306 | 5.090 | 5.019 |

TABLE 42
Measuring the red fluorescence of chip D (with nuclease and with SSB) for detecting the binding of
Cy ™ 5-labeled E. coli MBP mutS. The table gives the positions on the chip together with
the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 16.713 | 13.834 | 14.910 | 15.736 | 14.927 | 16.754 | 16.253 | 15.630 | 16.195 | 10.881 |
| 2 | 14.494 | 41.850 | 36.516 | 33.609 | 35.835 | 175.855 | 182.629 | 43.734 | 44.464 | 13.666 |
| 3 | 14.049 | 46.895 | 43.910 | 34.459 | 36.067 | 31.167 | 36.072 | 35.723 | 39.607 | 14.874 |
| 4 | 15.148 | 202.454 | 162.285 | 38.983 | 40.706 | 36.816 | 36.188 | 26.391 | 27.038 | 15.790 |
| 5 | 14.711 | 42.805 | 39.500 | 141.157 | 146.243 | 39.129 | 42.775 | 34.897 | 37.283 | 16.499 |
| 6 | 14.213 | 15.523 | 48.876 | 55.890 | 59.325 | 288.267 | 263.366 | 47.825 | 48.448 | 15.772 |
| 7 | 14.591 | 16.078 | 17.153 | 52.611 | 16.702 | 16.482 | 17.329 | 16.840 | 17.410 | 16.135 |
| 8 | 16.570 | 54.161 | >1049 | 16.312 | 19.006 | 16.076 | 16.105 | 49.142 | >1049 | 16.492 |
| 9 | 15.068 | 52.254 | >1049 | 18.827 | 17.997 | 18.335 | 16.739 | 52.521 | >1049 | 16.409 |
| 10 | 15.991 | 16.506 | 18.644 | 16.613 | 15.979 | 16.195 | 16.268 | 16.681 | 16.063 | 15.016 |

TABLE 43
Statistical analysis of the results obtained with hydrogel chips A-D.
The mean values and standard deviations of the red fluorescence
intensifies at all positions having the same loading were in each case
calculated for each chip.

| | | | Chip B: | Chip C: | Chip D: |
|---|---|---|---|---|---|
| | | Chip A: | without | with | with |
| | | without nuclease, | nuclease, | nuclease, | nuclease, |
| Capture | Target | without SSB | with SSB | without SSB | with SSB |
| cExon8 | MCF-7 | 469 +/− 51 | 197 +/− 15 | 38 +/− 4 | 37 +/− 3 |
| | MOLT-4 | 499 +/− 20 | 174 +/− 19 | 42 +/− 4 | 42 +/− 3 |
| | SW-480 | 558 +/− 55 | 255 +/− 13 | 152 +/− 14 | 168 +/− 21 |
| | 293 | 457 +/− 46 | 191 +/− 27 | 39 +/− 8 | 37 +/− 4 |
| ssPCR | MCF-7 | 473 +/− 34 | 123 +/− 12 | 56 +/− 0 | 51 +/− 2 |
| | MOLT-4 | 361 +/− 17 | 117 +/− 7 | 55 +/− 3 | 57 +/− 2 |
| | SW-480 | 408 +/− 28 | 284 +/− 6 | 260 +/− 5 | 275 +/− 13 |
| | 293 | 394 +/− 5 | 67 +/− 1 | 54 +/− 2 | 48 +/− 0 |

FIG. 18 shows the results obtained with hydrogel chip D (with nuclease and with SSB) when using the synthetic oligonucleotide cExon8 as the first strand. The figure in each case depicts the mean red fluorescence intensity, together with standard deviation, for the individual cell lines.

FIG. 19 shows the results obtained with hydrogel chip D (with nuclease and with SSB) when using the single-stranded PCR product “ssPCR” as the first strand. The figure in each case depicts the mean red fluorescence intensity, together with standard deviation, for the individual cell lines.

A comparison between the green fluorescence intensities at the positions to which the synthetic 73-mer oligonucleotide had been addressed, as the first strand, and the positions which were loaded with single-stranded PCR product shows that approximately the same amount of Cy™3-labeled second strand was bound in both cases. Overall, the positions which were loaded with the PCR product from the cell line MOLT-4 exhibited somewhat lower green fluorescences than did the positions which were loaded with PCR products from the other cell lines. This can be attributed to the fact that the PCR material from the cell line MOLT-4 which was employed for loading the chip contained less DNA than did the PCR products from the remaining cells. The marked decrease in the green fluorescence values which were measured in chips C and D following the treatment with single-strand-specific nuclease indicate that the degradation of the protruding single-stranded ends worked well.

When the red fluorescence values (Table 43) were analyzed, it was found that the mutation in Exon 8 of the p53 gene from the cell line SW-480 was only very weakly recognized in the case of chip A, which had not been treated either with nuclease or with single strand DNA-binding protein (SSB). A marked reduction in the red background fluorescence, and an improved mutation recognition, was already achieved with chip B, which was treated with SSB.

However, by comparison, chips C and D, which had been subjected to treatment with mung bean nuclease, exhibited a far lower background fluorescence and considerably better mutation recognition: with these chips, fluorescences were obtained which were 4 to 5 times higher for the mutation-carrying cell line SW-480 than they were for the cell lines MCF-7, MOLT-4 and 293, which exhibit the wild-type sequence in Exon 8 of p53 (Table 43, FIGS. 18 and 19). The additional treatment with SSB (chip D) resulted in a further slight improvement in the results (Table 43). In summary, this experiment showed that treatment with mung bean nuclease is very advantageous for the mutS-mediated detection of mutations in genomic DNA on electronically addressable microchips. In addition to this, incubation with SSB also has a positive effect on mutation recognition.

A comparison of the two methods, which are described here, for mutS-mediated mutation recognition shows that both methods are very well suited for detecting mutations in genomic DNA. When the single-stranded PCR product was used as the “capturing agent” (FIG. 19), a mutS signal was obtained which was even somewhat stronger than that obtained when using the shorter, synthetic oligonucleotide as the first strand (FIG. 18).

The greatest advantage when using biotinylated, single-stranded PCR products as “capturing agents” consists in the fact that it is possible, in this way, to test longer DNA regions for mutations than can be tested when using synthetic oligonucleotides. In addition to this, it is only when using this method that it is possible to compare genes or exons from two individuals with each other directly and without previous sequencing, i.e. by using the DNA from one of the individuals as the “capturing agent” and the sequence from the other individual as the “target”. In this way, it is possible, for example, to directly compare DNA sequences from patients suffering from a particular disease with DNA sequences from healthy control subjects.

On the other hand, however, the use of synthetic oligonucleotides as the first strand also offers some advantages: such oligonucleotides can be prepared in relatively large quantities and with any arbitrary sequence; in addition, synthetic oligonucleotides are already single-stranded, which means that it is not necessary to separate off the complementary strand. In addition to this, relatively short oligonucleotides can be used to delimit the position of a mutation which is possibly present more precisely than is the case when using a relatively long PCR product as the first strand.

Therefore, one or the other of the two protocols for the mutS-mediated detection of mutations in genomic DNA which are described here may prove particularly suitable, depending on the nature of the particular problem.

### Example: Use of mutS to Optically Recognize Base Mispairings

This experiment demonstrates how well the detection of mutations using the Cy™5-labeled E. coli mutS can be monitored optically. This is important with a view to using the technology for detecting mutations in multiple genes or patients. In this connection, good pattern recognition markedly facilitates the detection of mutations. For the chip-based detection of mutations, the following types of DNA double strands wer produced by hybridization on the different positions of an electronically addressable hydrogel chip suppli d by Nanogen:

completely complementary double strands

double strands which contain the GT base mispairing.

For this, the first-strand and second-strand oligonucleotides were firstly dissolved, at a concentration of 100 nM, in histidine buffer and denatured at 95° C. for 5 min. The biotinylated “sense” oligonucleotide (Seq. ID No. 10) was electronically addressed to the individual positions on the hydrogel chip, for 60 sec. at a voltage of 2.1 V, in the Nanogen workstation loading appliance. The hybridization with the second-strand AT (Seq. ID No. 12) and GT (Seq. ID No. 13) oligonucleotides was carried out for 120 sec at 2.1 V. The loading scheme is shown in Table 44; the name of each second-strand oligonucleotide indicates the mispairing which is formed on hybridization.

After loading, the chip was taken out of the loading appliance, filled with 1 ml of blocking buffer and incubated at room temperature for 60 min in order to saturate nonspecific protein-binding sites. The chip was subsequently incubated, at room temperature for 60 min, with 10 μl of Cy™5-labeled E. coli-mutS (concentration: 50 ng/μl) in 100 μl of incubation buffer. After this incubation, the chip was washed manually with 10 ml of washing buffer and then inserted into the Nanogen reader. In this reader, at a temperature of 37° C., it was washed 70× with in each case 0.5 ml of washing buffer.

Finally, the Cy™3 and Cy™5 fluorescence intensities at the individual positions on the chips were measured in the Nanogen reader using the following instrument settings: high sensitivity (“high gain”) for Cy™5, low sensitivity (“low gain”) for Cy™3; 256 μs integration time. The optical display of the fluorescence intensities took place automatically, after the measurement, on the Nanogen workstation using the e-Lab program.

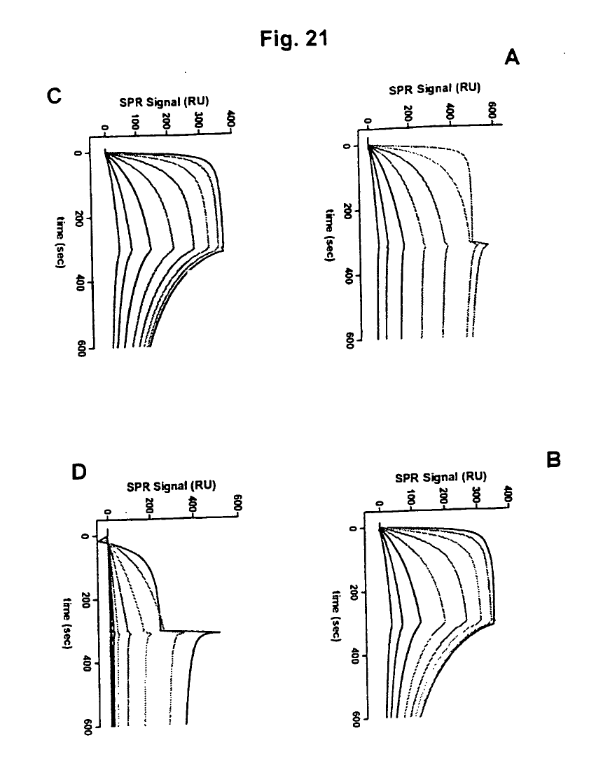

It can readily be seen from FIG. 20A that the chip was uniformly loaded with DNA. The mutations can be clearly recognized (FIG. 20B). Consequently, the mutS chip system is suitable for the rapid optical detection of mutations.

Histidine buffer: 50 mM L-histidine; this solution was filtered through a membrane having a pore size of 0.2 μm and degassed by negative pressure

Blocking buffer: 20 mM tris, pH 7.6/50 mM KCl/5 mM MgCl₂/0.01% Tween-20/3% BSA (Serva, Heidelberg)

Incubation buffer: 20 mM tris, pH 7.6/50 mM KCl/5 mM MgCl₂/0.01% Tween-20/1% BSA

Washing buffer: 20 mM tris, pH 7.6/50 mM KCl/5 mM MgCl₂/0.1% Tween-20

TABLE 44
Scheme for loading a chip for optically detecting the binding of
Cy ™ 5-labeled E. coli mutS to mispairings: all the
positions were firstly addressed with the “sense”
oligonucleotide (Seq. ID No. 10) and then the “AT”
(Seq. ID No. 12) and “GT” (Seq. ID No. 13)
oligonucleotides were addressed to the positions indicated.

| Position | 1 | 2 | 3 | 4 | 5 | 6 | 7 | 8 | 9 | 10 |
|---|---|---|---|---|---|---|---|---|---|---|
| 1 | AT | AT | AT | AT | AT | AT | AT | AT | AT | AT |
| 2 | AT | AT | AT | AT | AT | AT | AT | AT | AT | AT |
| 3 | AT | AT | AT | AT | AT | AT | AT | AT | AT | AT |
| 4 | GT | GT | GT | GT | AT | GT | AT | AT | AT | GT |
| 5 | GT | AT | AT | GT | AT | GT | AT | AT | GT | AT |
| 6 | GT | AT | AT | GT | AT | GT | AT | GT | AT | AT |
| 7 | GT | GT | GT | GT | AT | GT | GT | AT | AT | AT |
| 8 | GT | AT | AT | GT | AT | GT | AT | GT | AT | AT |
| 9 | GT | AT | AT | GT | AT | GT | AT | AT | GT | AT |
| 10 | GT | AT | AT | GT | AT | GT | AT | AT | AT | GT |

FIG. 20 shows the optical detection of mutations on electronically addressable DNA chips: FIG. 20A, the Cy™3 fluorescence indicates uniform loading of the chip with DNA, FIG. 20B, the Cy™5 fluorescence can be seen clearly at the positions possessing the base mispairing (see Table 44) and contrasts well with the background.

### Example: Recognition of Base Mispairings by Different mutS Proteins

This experiment examined whether the MBP-MutS prepared in the context of this application, the E. coli mutS (obtained from Gene Check, Fort Collins, Colo., USA), the Thermus aquaticus mutS (I. Biswas and P. Hsieh, J. Biol. Chem 271, 5040-5048 (1996), purchased from Biozym (Hess.-Oldendorf, Germany)) and the Thermus thermophilus HB8 mutS protein (S. Takamatsu, R. Kato and S. Kuramitsu, Nucl. Acids Res. 24, 640-647 (1996), kindly provided by Professor Kuramitsu, Osaka University, Japan) in each case had other properties with regard to recognizing the different possible base mispairings and insertions in DNA molecules. If, for example, one of the proteins binds preferentially to a base mispairing, would the binding to an unknown sequence restrict the nature of the base mispairing?

In order to check this, an investigation was carried out to determine whether different fluorescent dye-labeled mutS proteins bound particular base mispairings or insertions and deletions preferentially. For this, in each case 1 mg of the proteins was incubated, at room temperature for 30 min and in the dark, with 125 nmol Cy™5-succinimidyl ester in 10 ml of 10 mM HEPES pH 7.9, 50 mM KCl, 5 mM MgCl₂, 10% glycerol, 0.1 mM PMSF. Subsequently, the proteins were in each case loaded onto a 1 ml DEAE-sepharose fast flow column (Pharmacia, Sweden) which had been equilibrated with 10 ml of 10 mM HEPES pH 7.9, 50 mM KCl, 5 mM MgCl₂, 10% glycerol, 0.1 mM PMSF. Free active dye ester was removed by rinsing the column with 20 ml of 10 mM HEPES pH 7.9, 50 mM KCl, 5 mM MgCl₂, 10% glycerol, 0.1 mM PMSF and the protein was eluted in 4 ml of 10 mM HEPES pH 7.9, 500 mM KCl, 5 mM MgCl₂, 10% glycerol, 0.1 mM PMSF. After the purification, the integrity of the proteins was analyzed by SDS-PAGE. After the chromatographic purification, the proteins were dialyzed twice, for at least 3 hours, against 2 l of 10 mM HEPES pH 7.9, 50 mM KCl, 5 mM MgCl₂, 10% glycerol, 0.1 mM PMSF and then stored in 25 μl aliquots at −80° C.

In order to determine the degree of the fluorescence labeling (D/P ratio) of the different Cy™5-labeled mutS proteins, the protein concentrations of the different mutS proteins were first of all determined using the Bradford method. Depending on the protein concentration (0.1-1 mg/ml), the protein solution (1-10 μl) is made up with water (total volume=100 μl), after which Bradford reagent (900 μl, BioRad) is added. The formation of the protein-dye complex is complete after 15 min at room temperature. After the absorption has been measured at λ=595 nm, the protein concentration is determined with the aid of the calibration curve (constructed using BSA).

The resulting values for the individual mutS species are compiled in the following table:

| Protein | M_{w} (Da) | c (mg/ml) | c (μM) |
|---|---|---|---|
| mutS (E.coli) | 95246 | 0.15 | 1.57 |
| mutS (T. thermophilus) | 91249 | 0.24 | 2.63 |
| mutS (T. aquaticus) | 90627 | 0.15 | 1.66 |
| MBP-mutS (E. coli) | 137246 | 0.5 | 3.64 |

The concentration of Cy™5 dye was then determined by UV spectrometry. For this, buffer (950 μl, 10 mM HEPES pH 7.9, 50 mM KCl, 5 mM MgCl₂, 10% glycerol, 0.1 mM PMSF) was added to the protein solution (50 μl) and the Cy™5 absorption was then measured at λ=650 nm. The concentration of Cy™5 dye is now calculated as follows:

c(Cy™5)=(A₆₅₀)/250000 M⁻¹ cm⁻¹ (A₆₅₀=absorption at 650 nm).

The degree of fluorescence labeling (D/P ratio; D=Dye, P=protein) is now calculated as follows:

D/P=c(Cy™5)/c(mutS).

The resulting values for the individual mutS species are compiled in the following table:

| | M_{w} Monomer | c (mutS) | c(Cy ™5) | D/P |
|---|---|---|---|---|
| Protein | (Da) | [μM] | [μM] | ratio |
| mutS (E. coli) | 95246 | 1.57 | 0.48 | 0.31 |
| mutS | 91249 | 2.63 | 1.17 | 0.44 |
| (T. thermophilus) | | | | |
| mutS (T. aquaticus) | 90627 | 1.66 | 0.27 | 0.16 |
| MBP-mutS (E. coli) | 137246 | 3.64 | 0.53 | 0.15 |

The labeling efficiencies vary within one order of size.

A check was then carried out to determine how well the 4 different dye-labeled mutS proteins recognize different base mispairings or insertions. For this, the following types of DNA double strands were produced by hybridization at the different positions on electronically addressable hydrogel chips supplied by Nanogen:

completely complementary double strands,

double strands which contain one of the eight possible base mispairings (AA, AG, CA, CC, CT, GG, GT, TT),

double strands in which one strand contains an insertion of 1, 2 or 3 bases.

For this, the first-strand and second-strand oligonucleotides were first of all dissolved, at a concentration of 100 nM, in histidine buffer and denatured at 95° C. for 5 min. The biotinylated “sense” oligonucleotide (Seq. ID No. 10) was electronically addressed to the individual positions on the hydrogel chip, for 60 sec. and at a voltage of 2.1 V, in the Nanogen workstation loading appliance. The hybridization with the second-strand AT (Seq. ID No. 12), GT (Seq. ID No. 13), AA (Seq. ID No. 14), AG (Seq. ID No. 15), CA (Seq. ID No. 16), CC (Seq. ID No. 17), CT (Seq. ID No. 18), GG (Seq. ID No. 19), TT (Seq. ID No. 20), ins+1T (Seq. ID No. 21), ins+2T (Seq. ID No. 22) and ins+3T (Seq. ID No. 23) oligonucleotides was carried out for 120 sec. at 2.1 V. The loading scheme is shown in Table 45; the name of each second-strand oligonucleotide indicates the mispairing or insertion (“ins”) which is formed on hybridization.

After the loading, the chips were taken out of the loading appliance, filled with 1 ml of blocking buffer and incubated at room temperature for 60 min in order to saturate nonspecific protein-binding sites. The binding of E.coli mutS (ChipA), MBP-MutS (ChipB), Thermus aquaticus mutS (ChipC) and Thermus thermophilus mutS (Chip D) to the resulting DNA double strands was then tested. For this, the chips were incubated for 60 min with in each case 2-3 μg of the Cy™5-labeled mutS proteins in 100 μl of incubation buffer. The chips which were incubated with E.coli mutS and MBP mutS were incubated at room temperature while the chips which were incubated with Thermus aquaticus mutS and Thermus thermophilus mutS were incubated at 37° C. After this incubation, the chips were washed by hand with 10 ml of incubation buffer and inserted into the Nanogen reader. In the reader, the chips were washed, at a temperature of 37° C. (E.coli mutS and MBP-mutS) or 50° C. (Thermus aquaticus mutS and Thermus thermophilus mutS), 70-80× with in each case 0.25 ml of washing buffer.

Finally, the Cy™3 and Cy™5 fluorescence intensities at the individual positions on the chips were measured in the Nanogen reader using the following instrument settings: high sensitivity (“high gain”) in the case of Cy™5, low sensitivity (“low gain”) in the case of CY™3; 256 μs integration time. The values of the Cy™5 fluorescences for chips A-D are given in Tables 46-49. Care was taken to ensure that the Cy™3 fluorescence intensity was distributed homogeneously over the chip surface. The mean values of the Cy™5 fluorescences which were specific for the respective base mispairings are shown in Table 50.

In order to determine how well the individual proteins recognize the individual mispairings as compared with recognizing perfectly paired DNA (“AT”), the Cy™5 fluorescence attributed to this latter DNA was arbitrarily set at 1 and the other fluorescences were calculated on this basis (Tables 50 and 51).

In this connection, it was found that the two thermophilic proteins surprisingly bind particularly well to insertion mutations. They are therefore suitable for use in a system for exclusively detecting insertion/deletion mutations and G/T base mispairings.

In summary, it can be stated that both E. coli mutS and MBP-mutS are suitable for detecting a broad spectrum of base mispairings. In addition to this, the E. coli protein gives the most powerful signals in absolute terms. Interestingly, the proteins from T. thermophilus and T. aquaticus bind preferentially to mispairings which result from insertions/deletions. This can be used for rapidly detecting this mutation subtype.

Histidine buffer: 50 mM L-histidine; this solution was filtered through a membrane having a pore size of 0.2 μm and degassed by negative pressure

Blocking buffer: 20 mM tris, pH 7.6/50 mM KCl/5 mM MgCl₂/0.01% Tween-20/3% BSA (Serva, Heidelberg)

Incubation buffer: 20 mM tris, pH 7.6/50 mM KCl/5 mM MgCl₂/0.01% Tween-20/1% BSA

Washing buffer: 20 mM tris, pH 7.6/50 mM KCl/5 mM MgCl₂/0.1% Tween-20

TABLE 45
Scheme for loading chips for detecting the binding of Cy ™ 5-labeled mutS to different base
mispairings. “Neg.”: positions which were not loaded with DNA. “ssDNA”: positions which were only
loaded with the “senses” single strand. All the remaining positions were first of all addressed
with the “sense” oligonucleotide and then hybridized with the second strand given in the table.

| Position | 1 | 2 | 3 | 4 | 5 | 6 | 7 | 8 | 9 | 10 |
|---|---|---|---|---|---|---|---|---|---|---|
| 1 | AA | AA | AG | AG | AT | AT | CC | CC | AC | AC |
| 2 | CT | CT | ins + 1T | ins + 1T | ins + 2T | ins + 2T | TT | TT | GT | GT |
| 3 | GG | GG | GT | GT | GG | GG | Ins + 3T | ins + 3T | ssDNA | ssDNA |
| 4 | TT | TT | AC | AC | AT | AT | AG | AG | ins + 3T | ins + 3T |
| 5 | ssDNA | ssDNA | CC | CC | AA | AA | CT | CT | ins + 2T | ins + 2T |
| 6 | Neg. | Neg. | Neg. | Neg. | AG | AG | GG | GG | ins + 1T | ins + 1T |
| 7 | ssDNA | ssDNA | AT | AT | CC | CC | AC | AC | GT | GT |
| 8 | ins + 3T | ins + 3T | AG | AG | TT | TT | AA | AA | CT | CT |
| 9 | ins + 2T | ins + 2T | AA | AA | AC | AC | AT | AT | CC | CC |
| 10 | GG | GG | CT | CT | ins + 1T | Ins + 1T | GT | GT | TT | TT |

TABLE 46
Measuring the red fluorescence of chip A for detecting the binding Cy ™ 5-labeled E. coli
mutS to different base mispairings. The table gives the positions on the chip together with
the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 566.642 | 780.264 | 209.697 | 173.651 | 120.620 | 120.372 | 125.842 | 169.293 | 15.708 | 16.137 |
| 2 | 269.756 | 315.622 | 599.268 | 401.021 | 459.745 | 520.870 | 152.746 | 154.548 | 58.554 | 17.814 |
| 3 | 825.540 | 858.970 | 1.048.60 | 1.048.60 | 535.879 | 573.675 | 135.250 | 132.051 | 71.573 | 16.924 |
| 4 | 143.428 | 146.514 | 209.934 | 165.362 | 99.013 | 106.273 | 188.921 | 211.682 | 42.586 | 18.089 |
| 5 | 61.129 | 766.412 | 119.006 | 94.417 | 369.735 | 368.753 | 223.515 | 282.204 | 64.137 | 17.459 |
| 6 | 65.957 | 86.153 | 84.454 | 72.652 | 161.685 | 171.219 | 521.027 | 719.115 | 66.274 | 19.227 |
| 7 | 56.141 | 59.303 | 130.930 | 109.728 | 103.513 | 99.195 | 183.051 | 223.538 | 17.716 | 20.007 |
| 8 | 113.441 | 121.183 | 214.258 | 186.217 | 114.331 | 115.465 | 403.533 | 418.097 | >1049 | 26.695 |
| 9 | 528.991 | 560.922 | 612.501 | 593.926 | 245.658 | 180.629 | 96.750 | 103.293 | >1049 | 32.944 |
| 10 | 771.854 | 736.446 | 306.721 | 316.474 | 715.742 | 646.623 | 1.048.60 | 1.048.60 | 25.376 | 22.368 |

TABLE 47
Measuring the red fluorescence of chip B for detecting binding of Cy ™ 5-labeled
MBP-mutS to different base mispairings. The table gives the positions on the chip
together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 159.236 | 155.084 | 105.437 | 98.672 | 47.097 | 48.515 | 59.864 | 67.809 | 104.491 | 110.005 |
| 2 | 139.016 | 131.596 | 99.861 | 99.058 | 108.453 | 124.927 | 81.325 | 82.259 | 700.694 | 791.733 |
| 3 | 252.447 | 247.512 | 696.696 | 618.032 | 209.349 | 217.654 | 60.499 | 63.450 | 130.476 | 92.652 |
| 4 | 74.916 | 72.264 | 96.088 | 89.511 | 46.625 | 46.284 | 109.041 | 94.436 | 81.297 | 71.962 |
| 5 | 73.443 | 128.017 | 60.277 | 60.806 | 138.787 | 130.591 | 113.036 | 120.668 | 125.216 | 143.676 |
| 6 | 13.082 | 18.194 | 12.375 | 11.770 | 95.220 | 101.326 | 172.968 | 246.309 | 103.048 | 116.440 |
| 7 | 67.430 | 60.610 | 45.398 | 45.852 | 50.388 | 53.365 | 79.050 | 85.754 | 687.048 | 780.522 |
| 8 | 54.979 | 51.865 | 97.331 | 105.772 | 71.022 | 73.179 | 130.456 | 144.018 | 123.816 | 127.686 |
| 9 | 111.717 | 102.614 | 137.968 | 146.890 | 99.366 | 94.640 | 42.341 | 42.592 | 62.637 | 64.590 |
| 10 | 212.005 | 215.285 | 130.894 | 136.285 | 121.801 | 117.081 | 767.818 | 770.689 | 74.523 | 79.343 |

TABLE 48
Measuring the red fluorescence of chip C for detecting the binding of Cy ™ 5-labeled
Thermus equaticus mutS to different base mispairings. The table gives the positions
on the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 18.515 | 18.714 | 19.171 | 18.663 | 16.605 | 18.927 | 15.921 | 16.958 | 21.250 | 20.332 |
| 2 | 59.257 | 58.415 | 424.356 | 384.494 | 628.186 | 617.401 | 20.363 | 19.103 | 524.509 | 488.530 |
| 3 | 25.364 | 27.125 | 460.910 | 426.369 | 29.237 | 28.394 | 23.530 | 18.616 | 520.465 | 9.106 |
| 4 | 17.547 | 20.095 | 21.342 | 20.595 | 17.936 | 17.279 | 17.661 | 19.798 | 18.060 | 16.790 |
| 5 | 8.070 | 540.102 | 19.725 | 18.058 | 19.146 | 17.953 | 49.882 | 53.816 | 491.424 | 507.599 |
| 6 | 8.765 | 12.970 | 11.142 | 10.505 | 18.169 | 18.181 | 24.292 | 32.331 | 309.786 | 321.805 |
| 7 | 8.180 | 9.512 | 17.726 | 17.427 | 17.255 | 17.626 | 19.642 | 21.504 | 354.029 | 325.412 |
| 8 | 14.735 | 16.274 | 17.170 | 16.886 | 19.643 | 19.379 | 21.224 | 21.332 | 48.875 | 48.516 |
| 9 | 384.059 | 422.162 | 19.082 | 19.352 | 24.217 | 19.755 | 17.035 | 16.594 | 15.727 | 14.842 |
| 10 | 26.061 | 26.981 | 49.172 | 50.219 | 251.816 | 195.587 | 298.609 | 265.781 | 14.634 | 15.015 |

TABLE 49
Measuring the red fluorescence of chip D for detecting the binding of Cy ™ 5-labeled
Thermus thermophilus mutS to different base mispairings. The table gives the
positions on the chip together with the appurtenant relative fluorescence intensities.

| Position | 1 | 2 | 3 | 4 | 5 | 6 | 7 | 8 | 9 | 10 |
|---|---|---|---|---|---|---|---|---|---|---|
| 1 | 258.330 | 235.174 | 246.226 | 234.193 | 247.788 | 275.268 | 250.197 | 260.859 | 271.996 | 265.191 |
| 2 | 686.213 | 618.218 | 1.048.60 | 1.048.60 | 1.048.60 | 1.048.60 | 280.768 | 287.081 | 1.048.60 | 1.048.60 |
| 3 | 309.751 | 276.067 | 1.048.60 | 1.048.60 | 382.528 | 363.949 | 340.834 | 355.346 | 1.048.60 | 296.978 |
| 4 | 230.128 | 263.395 | 266.943 | 256.016 | 307.213 | 317.528 | 321.020 | 327.681 | 408.519 | 341.792 |
| 5 | 244.225 | 1.048.60 | 273.102 | 284.211 | 300.511 | 312.129 | 733.931 | 735.738 | 1.048.60 | 1.048.60 |
| 6 | 161.845 | 185.824 | 179.045 | 184.912 | 308.278 | 315.698 | 416.482 | 408.754 | 1.048.60 | 1.048.60 |
| 7 | 259.941 | 269.404 | 300.864 | 299.804 | 297.431 | 276.878 | 300.586 | 323.188 | 1.048.60 | 1.048.60 |
| 8 | 290.051 | 317.545 | 283.106 | 269.970 | 299.726 | 299.657 | 330.174 | 336.419 | 717.534 | 726.241 |
| 9 | 1.048.60 | 1.048.60 | 313.599 | 305.790 | 315.252 | 327.397 | 343.814 | 329.648 | 305.644 | 284.607 |
| 10 | 296.745 | 316.297 | 589.948 | 642.970 | 1.048.60 | 1.048.60 | 1.048.60 | 1.048.60 | 281.527 | 289.914 |

TABLE 50
Mean values of the Cy ™ 5 fluorescence values,
which are specific for the respective base
mispairings, of the individual mutS proteins.
The table shows the mean values for chips A-D

| | MBP- | E. coli | T. aquaticus | T. thermophilus |
|---|---|---|---|---|
| | mutS | mutS | mutS | mutS |
| AA | 129 | 437 | 9 | 121 |
| AC | 81 | 192 | 10 | 112 |
| AG | 87 | 112 | 7 | 110 |
| AT | 32 | 33.5 | 7 | 124 |
| CC | 46 | 46 | 6 | 101 |
| CT | 114 | 209 | 41 | 503 |
| GG | 208 | 616 | 16 | 168 |
| GT | 713 | 972 | 382 | 871 |
| TT | 62 | 61 | 7 | 101 |
| +1T | 96 | 552 | 304 | 871 |
| +2T | 108 | 567 | 503 | 871 |
| +3T | 50 | 61 | 7 | 164 |

TABLE 51
Relative fluorescence values for the binding of different
mutS proteins to the different base mispairings.
This table depicts the values given in Table 50
as related to the “AT” perfect pairing (=1.00).

| | MBP- | E. coli | T. aquaticus | T. thermophilus |
|---|---|---|---|---|
| | mutS | mutS | mutS | mutS |
| AA | 4.03 | 13.04 | 1.28 | 0.97 |
| AC | 2.53 | 5.73 | 1.43 | 0.90 |
| AG | 2.72 | 3.34 | 1.00 | 0.88 |
| AT | 1.00 | 1.00 | 1.00 | 1.00 |
| CC | 1.44 | 1.37 | 0.86 | 0.81 |
| CT | 3.56 | 6.24 | 5.85 | 4.0 |
| GG | 6.50 | 18.39 | 2.28 | 1.35 |
| GT | 22.28 | 29.02 | 54.57 | 7.02 |
| TT | 1.94 | 1.82 | 1.00 | 0.81 |
| +1T | 3.00 | 16.48 | 43.43 | 7.02 |
| +2T | 3.37 | 16.93 | 71.85 | 7.02 |
| +3T | 1.56 | 1.82 | 1.00 | 1.32 |

### Comparison Example: Using Biacore Measurement of the DNA/Protein Interaction to Investigate the Recognition of Base Mispairings by Different mutS Proteins

In order to investigate the DNA binding of mutS derived from different organisms, mutS derived from E.coli, T. aquaticus and T. thermophilus, and the MBP-mutS fusion protein, were tested by means of performing Biacore measurements. For this, use was made of the nucleotide sequences Seq. ID No. 11 to 23 for preparing heteroduplexes as were loaded onto the chip shown in Table 45. The K_{d} values specific for the individual base mispairings were then determined using surface plasmon resonance.

The analyses were carried out on a Biacore2000 SPR Biosensor (Biacore AB) at 22° C. in a running buffer consisting of 20 mM HEPES (pH7.4), 50 mM KCl, 5 mM MgCl₂ and 0.005% Tween20 (protein grade, Calbiochem). The DNA oligonucleotides were immobilized on a streptavidin-coated surface of an SA sensor chip (Biacore AB) up to a surface density of 70 RU. An SA surface without DNA served as the control surface. The proteins were diluted in running buffer in order to obtain a concentration series of eight different concentrations of the respective protein, which concentrations were led consecutively over the sensor surfaces. The binding of the protein to the DNA, as well as the dissociation, was in each case detected for 5 min at a flow rate of 10 μl/min. After each binding operation, the surfaces were regenerated with 2 consecutive injections of 0.1% SDS (in each case 0.5 min, flow rate 30 μl/min) before the next concentration of the protein was injected.

The data were analyzed using the Biaevaluation software version 3.1. The signals for the control surface were subtracted from the signals for the individual surfaces and the curves were normalized to the injection start. The association and dissociation were determined either separately or by way of a global fit using a Langmuir 1:1 binding model. The affinities (K_{D} values) were calculated from the formula K_{D}=k_{diss}/kₐₛₛ. In the case of kinetics which equilibrium was established very rapidly, the signals at equilibrium (R_{eq}) were plotted against the concentration of the protein and the K_{D} values were determined by way of an hyperbolic fit.

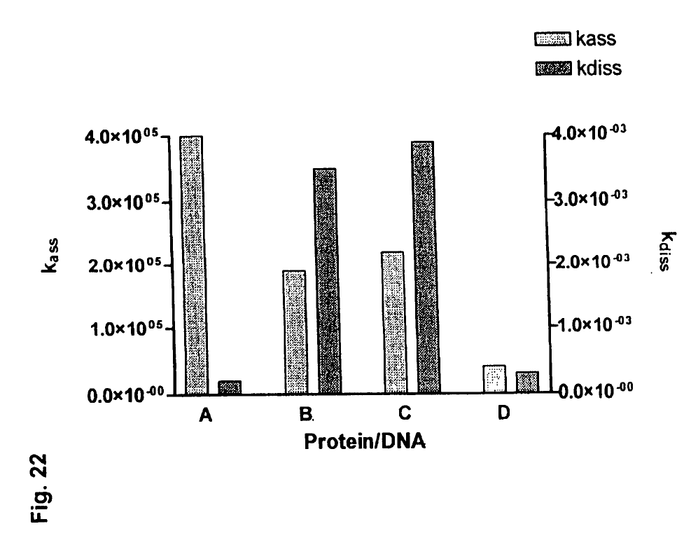

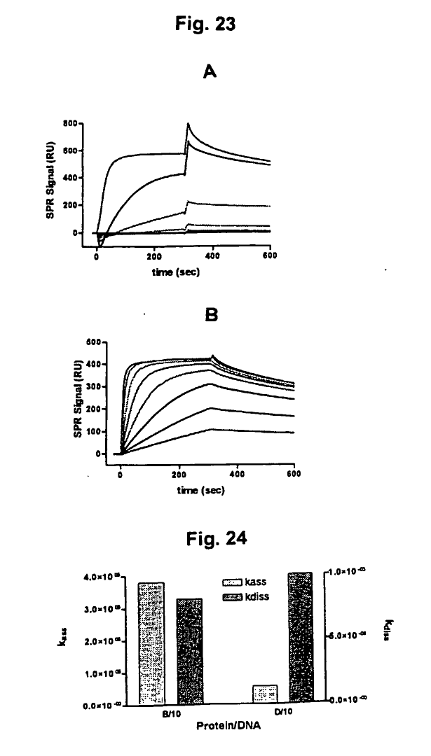

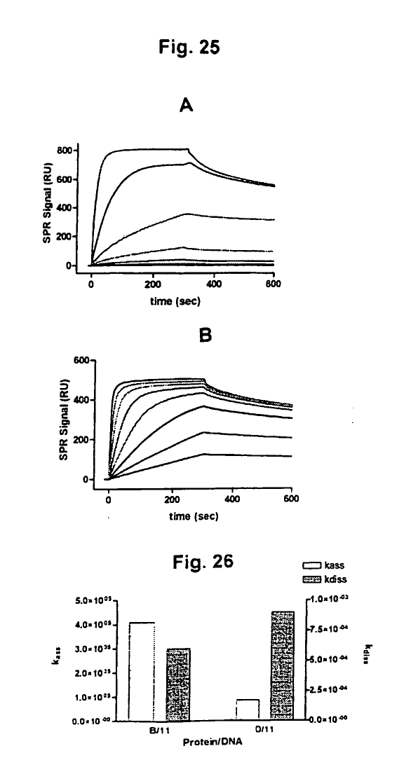

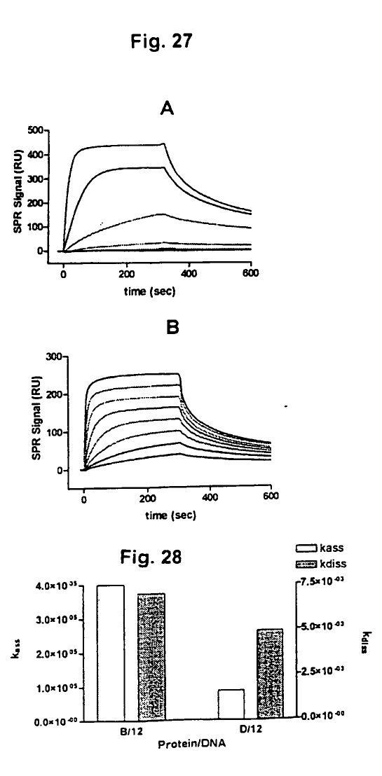

The resonance values which were determined from the Biacore measurements agreed, without exception, with the results from the chip experiment (Tables 50 and 51). By way of example, the binding of the GT mispairing in the case of the four mutS variants (A: E.coli, B: T. thermophilus, C: T. aquatiqus and D: MBP-mutS) is shown in FIGS. 21A-D, while the graphic depiction of the association and dissociation constants is shown in FIG. 22. The high specificity of the T. thermophilus mutS protein for +1T (FIG. 23B), for +2T (FIG. 25B), and for +3T (FIG. 27B) as compared with MBP-mutS(E.coli) fusion protein (FIGS. 23A, 25A and 27A) is shown in FIGS. 23, 25 and 27; FIG. 24 shows the graphic depiction of the constants which were determined in the case of the +1T insertion, while FIG. 26 shows the graphic depiction in the case of the +2T insertion and FIG. 28 shows that in the case of the +3T insertion.

In ascending order, the measured graphs were recorded at the following mutS concentrations:

FIG. 21A: 2.5 nM, 5 nM, 10 nM, 20 nM 39 nM, 79 nM, 158 nM

FIG. 21B: 3.5 nM, 7 nM, 14 nM, 27 nM, 55 nM, 110 nM, 219 nM, 438 nM

FIG. 21C: 3.4 nM, 7 nM 14 nM 28 nM 55 nM, 110 nM 221 nM, 441 nM

FIG. 21D: 2.8 nM, 5.7 nM, 11 nM, 23 nM, 45 nM, 91 nM 182 nM 363 nM

FIGS. 23A, 25A, 27A: 5.7 nM, 11 nM, 23 nM, 45 nM 91 nM 182 nM, 363 nM, 726 nM

FIGS. 23B, 25B, 27B: 3.5 nM, 7 nM, 14 nM, 27 nM, 55 nM, 110 nM, 219 nM, 438 nM

### Example: Using Impedance Spectroscopy to Detect the Binding of mutS Protein to ds Oligonucleotide Monolayers

1. Preliminary Treatment of the Gold Electrodes

The Au electrodes (CH-Instruments, Austin, USA) were cleaned by polishing the electrode surface with an 0.3 μm alumina suspension (LECO, St. Joseph, USA) and subsequent rinsing with Millipore water (10 MΩ cm). The subsequent electrochemical cleaning of the electrodes was carried out by cyclovoltametry (potentiostat: EG&G PAR 273A, GB) in 0.2 M NaOH, with the electrodes first of all being cycled 5 times between potentials of −0.5 and −1.8 V (against Ag/AgCl-reference electrode (Metrohm GmbH & Co, Filderstadt, Germany) 3 M NaCl) and then 3 times between −0.3 and 1.1 V (feed rate 50 mV sec⁻¹). A platinum rod (Metrohm) was used as the counter electrode in the cyclovoltametry.

2. Binding of 5′-SH-oligonucleotides to Gold Surfaces

The 5′-SH-modified oligonucleotide hairpins (Interactiva, Ulm, Germany) (Seq. ID No. 50 and Seq. ID No. 51) were bound on by incubating the cleaned Au electrodes with a 100 μM solution of the corresponding thiol-modified oligonucleotide in 0.9 M phosphate buffer (pH 6.6, Calbiochem; addition of 0.5 mM dithiothreitol, (DTT, Sigma-Aldrich, Steinheim, Germany)) for 6.5 h.

The characterization of the resulting oligonucleotide monolayers was checked by blocking the diffusion-controlled Fe(II/III) redox reaction in aqueous K₃/K₄Fe(CN)₆ solution (salts from Merck, Darmstadt, Germany) (20 mM in 20 mM phosphate buffer, pH 7), using cyclic voltametry (CV).

3. Filling the Monolayer Interstices with 1.6-mercaptohexanol

The interstices between the individual oligonucleotide molecules on the monolayer were filled by incubating the electrodes in a 1 mM solution of 6-mercaptohexanol (Aldrich, USA) in Millipore water (degassed) at room temperature for 60-90 minutes. Until the actual measurement, the electrodes were stored at 4° C. in 1 M phosphate buffer.

4. Incubating with mutS Protein

For this, the individual electrodes coated with hairpin oligonucleotides were incubated, at room temperature for more than 30 minutes, with approx. 20 μl of 20 mM tris buffer (100 mM KCl, 5 mM MgCl₂, pH 7.6). Before the application, ⅕ of the buffer volume was replaced with mutS concentrate (0.5 mg/ml).

5. Electrochemical Experiments

The individual electrodes were measured in aqueous K₃/K₄Fe(CN)₆ solution (Darmstadt, Germany) (20 mM in 20 mM tris buffer, 100 mM KCl, 5 mM MgCl), in the frequency range from 100 mHz to 1 MHz to 100 mHz, before and after incubating with mutS. The measured values are shown in the Bode diagram. The measurements were carried out, at room temperature, on an IM 6e impedance measuring desk supplied by Zahner Messtechnik.

Nucleic acid sequence: 5′-3′ X=aminomodifier-dT mutS substrate

| | |
|---|---|
| Seq. ID No. 50 | ATT CGA TCG GGG CGG GGC GAG CTT TTX |
| | GCT CGC CTT GCC CCG ATC GAA T |
| Seq. ID No. 51 | ATT CGA TCG GGG CGG GGC GAG CTT XTT |
| | GCT CGC CCC GCC CCG ATC GAA T |

Impedance Spectroscopy

Impedance spectroscopy was used for the electrochemical investigation. In this method of investigation, an alternating voltage, whose frequency varies, is applied to the system to be investigated and, at the same time, the impedance is measured. Computer-assisted measuring desks, which simplify the management and operability of the investigation, and the reduced costs, in particular, have resulted in these measurement methods for investigating surfaces becoming widespread.

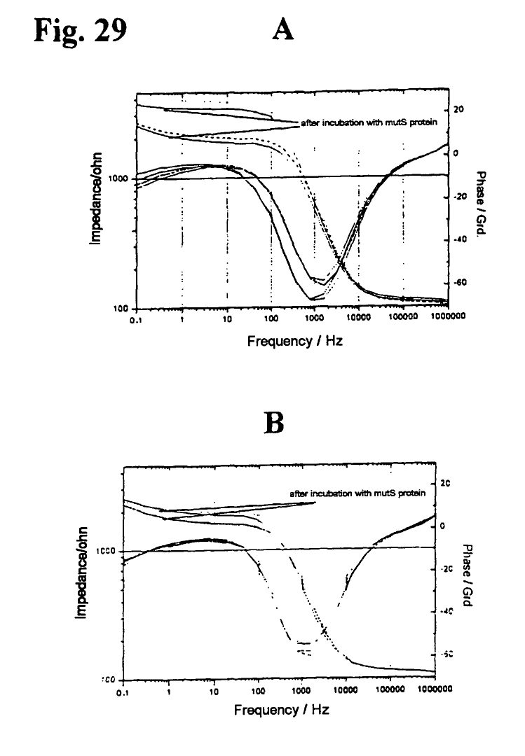

The advantage of this method is that it is possible to carry out measurements on the samples without destroying them and without using labels. The mutation-recognizing E. coli mutS protein was used as a model system for the binding of mispairing-recognizing substrates, while Seq. ID Nos. 50 and 51 were used as duplex-forming oligonucleotides. By introducing an aminomodifier building block into the hairpin loop, the affinity of mutS to bind at this site was selectively suppressed. The results of the measurement are depicted in FIGS. 29A and 29B. The broken lines show the impedance before adding mutS while the unbroken lines show the impedance after adding mutS (the phase curves possess a minimum at approx. 1000 Hz). FIG. 29A shows two measurements of sequence Seq ID No. 50, which contains two GT mispairings, while FIG. 29B shows two measurements which were carried out using Seq ID No. 51, which does not contain these two base mispairings. In the present example, the decrease in the impedance in the range of below 100 Hz, which is of interest for the measurement, indicates that mutS binding has increased. As expected, FIG. 29A shows a decrease in the impedance as a result of the binding of mutS; no, or only very slight, binding of mutS can be seen in FIG. 29B.

### Example: Influence of the Concentration of the Oligonucleotides Employed on mutS-Mediated Mutation Detection

This example is intended to demonstrate that the detection of mutations using fluorescent dye-labeled E.coli mutS as the mispairing-recognizing substrate functions over a wide DNA concentration range. For this, the individual positions on a hydrogel chip were loaded with solutions containing different concentrations of perfectly pairing or GT-mispairing first-strand and second-strand oligonucleotides, and the binding of mutS to the respective positions was then determined.

Experimental Implementation:

The oligonucleotides employed were in each case dissolved, at several different concentrations (100 nM, 33 nM, 10 nM, 3.3 nM, 1 nM and 0.33 nM), in histidine buffer and denatured at 95° C. for 5 min. The solutions of different concentrations of the biotinylated “sense” first-strand oligonucleotide (Seq. ID No. 10) were electronically addressed to the individual positions on a hydrogel chip, for 60 sec. and at a voltage of 2.1 V, in the Nanogen workstation loading appliance. The hybridization with the solutions of different concentrations of the AT (Seq. ID No. 12) and GT (Seq. ID No. 13) counterstrands was carried out for 120 sec at 2.1 V. The loading scheme is shown in Table 52.

After the loading, the chip was taken out of the loading appliance, filled with 1 ml of blocking buffer and incubated at room temperature for 60 min in order to saturate nonspecific protein-binding sites. The chip was then incubated, at room temperature for 60 min, with 10 μl of Cy5-labeled E. coli mutS (concentration: 50 ng/μl) in 90 μl of incubation buffer. After this step, the chip was washed by hand with 1 ml of incubation buffer, then inserted into the Nanogen reader and washed, in the reader and at a temperature of 37° C., 50× with in each case 250 μl of washing buffer. Finally, the Cy™5 fluorescence intensity at the individual positions on the chip was measured in the Nanogen reader using the following instrument setting: high sensitivity (“high gain”); 256 μs integration time.

Histidine buffer: 50 mM L-histidine, filtered through an 0.2 μm membrane and degassed

Blocking buffer: 20 mM tris, pH 7.6/50 mM KCl/5 mM MgCl₂/0.01% Tween-20/3% BSA

Incubation buffer: 20 mM tris, pH 7.6/50 mM KCl/5 mM MgCl₂/0.01% Tween-20/1% BSA

Washing buffer: 20 mM tris, pH 7.6/50 mM KCl/5 mM MgCl₂/0.1 % Tween-20

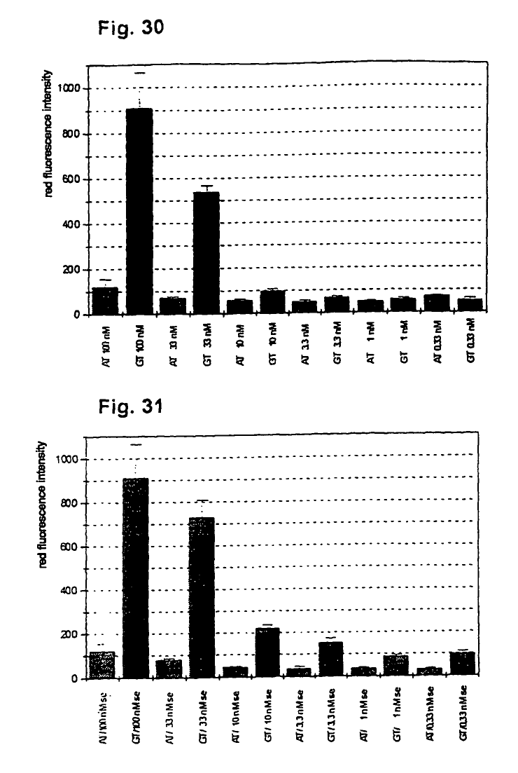

The red fluorescence intensities which were measured are listed in Table 53. The results of the statistical analysis of the measured values are summarized in Table 54 and illustrated in FIGS. 30-32.

TABLE 52
Scheme for loading a hydrogel chip with different concentrations of the first and second
strands. The individual positions were firstly loaded with the concentrations of the biotinylated “sense”
oligonucleotide which are in each case indicated and then hybridized with the second-strand “AT” or
“GT” oligonucleotides in the concentrations listed. As controls, some positions were only loaded with
first or second strand; as the reference electrode, position 6/2 remained free.

| Position | 1 | 2 | 3 | 4 | 5 | 6 | 7 | 8 | 9 | 10 |
|---|---|---|---|---|---|---|---|---|---|---|
| 1 | sense | sense | sense | sense | sense | sense | sense | sense | sense | sense |
| | 100 nM | 100 nM | 100 nM | 100 nM | 100 nM | 100 nM | 33 nM | 3.3 nM | 330 pM | 100 nM |
| | | AT | AT | AT | AT | AT | AT | AT | AT | |
| | | 100 nM | 33 nM | 3.3 nM | 1 nM | 100 nM | 100 nM | 100 nM | 100 nM | |
| 2 | sense | sense | sense | sense | sense | sense | sense | sense | sense | sense |
| | 100 nM | 100 nM | 100 nM | 100 nM | 100 nM | 100 nM | 33 nM | 3.3 nM | 330 pM | 3.3 nM |
| | | GT | GT | GT | GT | GT | GT | GT | GT | AT |
| | | 100 nM | 33 nM | 3.3 nM | 1 nM | 100 nM | 100 nM | 100 nM | 100 nM | 3.3 nM |
| 3 | sense | sense | sense | sense | sense | sense | sense | sense | sense | |
| | 3.3 nM | 100 nM | 100 nM | 100 nM | 100 nM | 100 nM | 10 nM | 3.3 nM | 330 pM | |
| | AT | AT | AT | AT | AT | AT | AT | AT | AT | AT |
| | 3.3 nM | 100 nM | 33 nM | 3.3 nM | 330 pM | 100 nM | 100 nM | 100 nM | 100 nM | 100 nM |
| 4 | sense | sense | sense | sense | sense | sense | sense | sense | sense | |
| | 3.3 nM | 100 nM | 100 nM | 100 nM | 100 nM | 100 nM | 10 nM | 3.3 nM | 330 pM | |
| | GT | GT | GT | GT | GT | GT | GT | GT | GT | GT |
| | 3.3 nM | 100 nM | 33 nM | 3.3 nM | 330 pM | 100 nM | 100 nM | 100 nM | 100 nM | 100 nM |
| 5 | sense | sense | sense | sense | sense | sense | sense | sense | sense | sense |
| | 33 nM | 3.3 nM | 100 nM | 100 nM | 100 nM | 100 nM | 10 nM | 1 nM | 330 pM | 10 nM |
| | AT | GT | AT | AT | AT | AT | AT | AT | AT | AT |
| | 33 nM | 3.3 nM | 10 nM | 3.3 nM | 330 pM | 100 nM | 100 nM | 100 nM | 100 nM | 10 nM |
| 6 | sense | | sense | sense | sense | sense | sense | sense | sense | sense |
| | 33 nM | | 100 nM | 100 nM | 100 nM | 100 nM | 10 nM | 1 nM | 330 pM | 10 nM |
| | GT | | GT | GT | GT | GT | GT | GT | GT | GT |
| | 33 nM | | 10 nM | 3.3 nM | 330 pM | 100 nM | 100 nM | 100 nM | 100 nM | 10 nM |
| 7 | sense | sense | sense | sense | sense | sense | sense | sense | | sense |
| | 33 nM | 100 nM | 100 nM | 100 nM | 100 nM | 33 nM | 10 nM | 1 nM | | 10 nM |
| | AT | AT | AT | AT | AT | AT | AT | AT | AT | AT |
| | 33 nM | 100 nM | 10 nM | 1 nM | 330 pM | 100 nM | 100 nM | 100 nM | 100 nM | 10 nM |
| 8 | sense | sense | sense | sense | sense | sense | sense | sense | | sense |
| | 33 nM | 100 nM | 100 nM | 100 nM | 100 nM | 33 nM | 10 nM | 1 nM | | 10 nM |
| | GT | GT | GT | GT | GT | GT | GT | GT | GT | GT |
| | 33 nM | 100 nM | 10 nM | 1 nM | 330 pM | 100 nM | 100 nM | 100 nM | 100 nM | 10 nM |
| 9 | sense | sense | sense | sense | sense | sense | sense | sense | | sense |
| | 33 nM | 100 nM | 100 nM | 100 nM | 3.3 nM | 33 nM | 3.3 nM | 1 nM | | 10 nM |
| | AT | AT | AT | AT | AT | AT | AT | AT | AT | AT |
| | 33 nM | 33 nM | 10 nM | 1 nM | 3.3 nM | 100 nM | 100 nM | 100 nM | 100 nM | 10 nM |
| 10 | sense | sense | sense | sense | sense | sense | sense | sense | | sense |
| | 33 nM | 100 nM | 100 nM | 100 nM | 3.3 nM | 33 nM | 3.3 nM | 1 nM | | 10 nM |
| | GT | GT | GT | GT | GT | GT | GT | GT | GT | GT |
| | 33 nM | 33 nM | 10 nM | 1 nM | 3.3 nM | 100 nM | 100 nM | 100 nM | 100 nM | 10 nM |

TABLE 53
Measuring the red fluorescence intensity of the hydrogel chip, which was loaded with
different concentrations of oligonucleotides, for the purpose of detecting the binding of Cy5-labeled
E. coli mutS. The table gives the positions on the chip together with the appurtenant relative
fluorescence intensities.

| Position | 1 | 2 | 3 | 4 | 5 | 6 | 7 | 8 | 9 | 10 |
|---|---|---|---|---|---|---|---|---|---|---|
| 1 | 75.105 | 164.910 | 70.523 | 56.769 | 51.529 | 113.043 | 78.859 | 30.587 | 29.999 | 77.899 |
| 2 | 71.100 | >1049 | 511.815 | 73.025 | 63.576 | 786.972 | 646.291 | 170.041 | 111.207 | 39.412 |
| 3 | 28.364 | 147.509 | 67.382 | 49.846 | 70.842 | 76.447 | 45.623 | 35.657 | 32.462 | 27.928 |
| 4 | 50.019 | >1049 | 540.599 | 64.273 | 58.336 | 728.072 | 205.950 | 159.541 | 92.634 | 101.742 |
| 5 | 77.246 | 52.097 | 62.225 | 45.900 | 64.422 | 90.401 | 42.199 | 34.951 | 34.979 | 69.642 |
| 6 | 625.025 | 19.997 | 109.412 | 65.994 | 47.718 | 806.710 | 207.836 | 93.941 | 87.221 | 102.658 |
| 7 | 65.984 | 135.205 | 53.654 | 45.992 | 73.829 | 81.680 | 46.963 | 39.515 | 34.075 | 50.993 |
| 8 | 553.442 | >1049 | 94.094 | 55.038 | 58.623 | 771.498 | 239.368 | 89.576 | 78.241 | 80.750 |
| 9 | 55.988 | 77.402 | 61.436 | 48.432 | 39.490 | 85.906 | 45.050 | 38.145 | 32.771 | 46.456 |
| 10 | 455.562 | 565.882 | 94.869 | 59.377 | 50.964 | 782.279 | 134.114 | 80.300 | 66.271 | 76.117 |

TABLE 54
Statistical analysis of the binding of mutS to
the hydrogel chip loaded with different
concentrations of oligonucleotides. The mean values
and standard deviations of the red fluorescence
intensity at all the positions having the same
loading were calculated in each case.

| First strand | Second strand | Result |
|---|---|---|
| sense 100 nM | AT 100 nM | 121.3 +/− 34.2 |
| sense 100 nM | AT 33 nM | 71.8 +/− 5.1 |
| sense 100 nM | AT 10 nM | 59.1 +/− 4.7 |
| sense 100 nM | AT 3.3 nM | 50.8 +/− 5.5 |
| sense 100 nM | AT 1 nM | 48.6 +/− 2.8 |
| sense 100 nM | AT 330 pM | 69.7 +/− 4.8 |
| sense 33 nM | AT 100 nM | 82.2 +/− 3.5 |
| sense 33 nM | AT 33 nM | 66.4 +/− 10.6 |
| sense 10 nM | AT 100 nM | 44.9 +/− 2.5 |
| sense 10 nM | AT 10 nM | 55.7 +/− 12.2 |
| sense 3.3 nM | AT 100 nM | 37.1 +/− 7.4 |
| sense 3.3 nM | AT 3.3 nM | 35.8 +/− 6.4 |
| sense 1 nM | AT 100 nM | 37.5 +/− 2.3 |
| sense 330 pM | AT 100 nM | 32.5 +/− 2.5 |
| sense 100 nM | GT 100 nM | 911.5 +/− 152.8 |
| sense 100 nM | GT 33 nM | 539.7 +/− 27.0 |
| sense 100 nM | GT 10 nM | 99.3 +/− 8.4 |
| sense 100 nM | GT 3.3 nM | 67.8 +/− 4.6 |
| sense 100 nM | GT 1 nM | 59.3 +/− 4.3 |
| sense 100 nM | GT 330 pM | 54.9 +/− 6.2 |
| sense 33 nM | GT 100 nM | 733.0 +/− 75.5 |
| sense 33 nM | GT 33 nM | 544.7 +/− 84.8 |
| sense 10 nM | GT 100 nM | 217.7 +/− 18.5 |
| sense 10 nM | GT 10 nM | 86.6 +/− 14.4 |
| sense 3.3 nM | GT 100 nM | 154.7 +/− 18.6 |
| sense 3.3 nM | GT 3.3 nM | 51.0 +/− 1.1 |
| sense 1 nM | GT 100 nM | 87.9 +/− 7.0 |
| sense 330 pM | GT 100 nM | 96.9 +/− 12.5 |

FIG. 30 shows the second-strand dilution series. The concentration of the “sense” oligonucleotide used as the first strand was in each case 100 nM; the AT or GT oligonucleotide used as the second strand were employed in the concentrations given in the diagram. The figure in each case shows the mean red fluorescence intensity, following mutS binding, for the individual second-strand concentrations.

FIG. 31 shows the first-strand dilution series. The “sense” oligonucleotide used as the first strand was employed in the concentrations given in the diagram. The concentrations of the AT or GT oligonucleotide used as the second strand were in each case 100 nM. The figure shows the mean red fluorescence intensity, following mutS binding, for the individual first-strand concentrations.

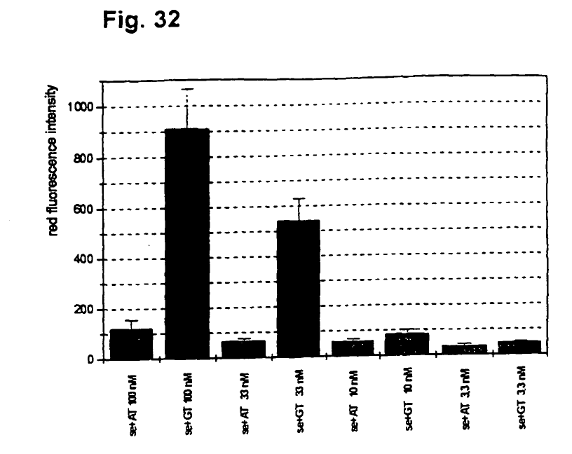

FIG. 32 shoes the simultaneous dilution of the first and the second strand. The “sense” oligonucleotide used as the first strand, and also the AT or GT oligonucleotide used as the second strand, were diluted equally; the concentrations employed are given in the diagram. The figure shows the mean red fluorescence intensity, following mutS binding, for the individual oligonucleotide concentrations.

It is evident from FIG. 30 that the concentration of the second-strand oligonucleotides employed can be decreased significantly as compared with the concentration of 100 nM which was used in the previously described experiments: When 33 nM second-strand solutions are used, mutS binds by a factor of 7.5 more strongly to the GT mispairing than it does to the perfect pairing (AT), and the GT mispairing is still recognized by a factor of 1.7 compared with the perfect pairing when 10 nM second-strand solutions are used. If, on the other hand, the concentration of the first-strand solution employed is lowered while the second-strand concentration remains constant at 100 nM, mutS still binds by a factor of 2.3 more strongly to the GT mispairing than it does to the perfect pairing even at a first-strand concentration of only 1 nM (FIG. 31). In addition to this, obvious mutS-mediated recognition of the GT mispairing was still achieved even after the concentrations of the first-strand DNA and second-strand DNA had been simultaneously lowered to 33 nM (FIG. 32).

It can thus be demonstrated that relatively large variations in the concentration of the DNA employed do not result in correspondingly large variations in mutS binding. This demonstrates the high degree of reliability of the method for detecting mutations which is described here, in particular in the range of practically relevant DNA concentrations as are obtained when examining samples derived from patients. Furthermore, this example shows that a comparison of different patient samples is also possible when the individual samples do not have exactly the same DNA concentration. In addition, the experiment demonstrated that even very small quantities of DNA are adequate for detecting mutS-mediated mutations.

1. A method for detecting mutations in nucleotide sequences comprising the procedural steps of hybridizing single-stranded sample nucleotide sequences with single-stranded reference nucleotide sequences, fixing single-stranded reference nucleotide sequences or single-stranded sample nucleotide sequences before or during the hybridization, or heteroduplexes consisting of reference and sample nucleotide sequences after or during the hybridization, on a support in a site-resolved manner, incubating with a substrate which recognizes heteroduplex mispairings, and detecting the substrate bindings.

2. A method for detecting mutations in nucleotide sequences, wherein
a) a defined, single-stranded nucleotide sequence is loaded onto a nucleotide chip,
b) the nucleotide sequence which is to be examined for mutation, and which is complementary to the known nucleotide sequence, is likewise loaded onto the chip and a heteroduplex is produced by hybridizing the two sequences,
c) the heteroduplex is incubated with a labeled substrate which recognizes mispairings, and
d) the mispairings are detected by detecting the labeled substrate which is attached to them.

3. The method as claimed in claim 1 or 2, wherein the single-stranded nucleotide sequences which are fixed on the support and which are not hybridized are degraded by adding a nuclease.

4. The method as claimed in claim 3, wherein the nuclease employed is mung bean nuclease or S1 nuclease.

5. The method as claimed in claim in one of claims 1 to 4, wherein the support employed is an electronically addressable surface.

6. The method as claimed in claim 5, wherein the fixing and/or hybridization is effected in an electronically accelerated manner.

7. The method as claimed in claim 5 or 6, wherein a site-resolved, electronically accelerated hybridization is carried out, with the hybridization conditions being set individually at the respective site.

8. The method as claimed in claim 7, wherein the individual setting of the hybridization conditions is effected by the current strength which is applied at the respective site, the voltage which is applied at the respective site or the duration of the electronic addressing.

9. The method as claimed in one of claims 1 to 8, wherein the electronically addressable surface employed is a nucleotide chip.

10. The method as claimed in one of claims 1 to 9, wherein use is made of an electronically addressable surface which is coated with a permeation layer.

11. The method as claimed in claim 10, wherein the permeation layer possesses a high degree of permeability for nucleotide sequences and the substrates which recognize heteroduplex mispairings.

12. The method as claimed in claim 10 or 11, wherein the permeation layer employed is a hydrogel layer.

13. The method as claimed in one of claims 1 to 12, wherein the incubation with the substrate is effected under low salt conditions.

14. The method as claimed in claim 13, wherein the incubation with the substrate is effected at a salt concentration of between 25 mM and 75 mM.

15. The method as claimed in one of claims 1 to 14, wherein BSA is added prior to the incubation with the mispairing-recognizing substrate.

16. The method as claimed in one of claims 1 to 15, wherein SSB is added prior to incubation with the mispairing-recognizing substrate.

17. The method as claimed in one of claims 1 to 16, wherein use is made of a mispairing-recognizing substrate which is selected from the group consisting of the mispairing-binding proteins.

18. The method as claimed in claim 17, wherein the mispairing-recognizing substrate employed is a protein selected from the group consisting of the mutS proteins, mutY proteins, MSH 1 to 6 proteins, S1 nuclease, T4 endonuclease, thymine glycosylase or cleavase, or a mixture of these proteins.

19. The method as claimed in claim 18, wherein the mispairing-binding protein is the mutS protein from E.coli, from T. thermophilus or from T. aquaticus.

20. The method as claimed in one of claims 1 to 19, wherein a labeled substrate which recognizes mispairings is employed.

21. The method as claimed in one of claims 1 to 20, wherein use is made of a radioactively labeled, luminescent, dye-labeled or fluorescence-labeled substrate which recognizes mispairings or of a substrate which recognizes mispairings and which is provided with quantum dots or with a polymeric label or metal label.

22. The method as claimed in claim 21, wherein the substrate employed is labeled with Cy™3, Cy™5, Oregon Green 488, Alexa Fluor 488, Alexa Fluor 532, Alexa Fluor 546, Alexa Fluor 594, Alexa Fluor 647, Bodipy 558/568, Bodipy 650/665, Bodipy 564/570, S 0535, S 0536, Dy-630-NHS, Dy-635-NHS, EVOblue30-NHS, FAR-Blue, FAR-Fuchsia, Atto 650, FITC or Texas Red.

23. The method as claimed in one of claims 1 to 22, wherein use is made of a substrate fusion protein which recognizes mispairings.

24. The method as claimed in claim 23, wherein the fused domain of the substrate fusion protein employed is an epitope for an antibody binding or possesses an enzymic activity.

25. The method as claimed in one of claims 1 to 24, wherein the reference nucleotide sequence and/or the sample nucleotide sequence is/are radioactively labeled, luminescence-labeled, dye-labeled, fluorescence-labeled, quantum dots-labeled, polymer-labeled or metal-labeled.

26. The method as claimed in one of claims 1 to 25, wherein, instead of the base mispairings which are weakly bound by the mispairing-recognizing substrate, use is made of their corresponding mispairings.

27. The method as claimed in claim 26, wherein a mixture of heteroduplexes containing mispairings which correspond to each other is incubated with a mispairing-recognizing substrate.

28. The method as claimed in claim 26 or 27, wherein, when a mutS protein is used as the mispairing-recognizing substrate, use is made, for the substrate binding, of the mispairing GG in place of the mispairing CC, of the mispairing AA in place of the mispairing TT, and/or of the mispairing GT in place of the mispairing AC, or of a mixture of heteroduplexes carrying mispairings from this group which correspond to each other.

29. The method as claimed in one of the preceding claims, wherein the detection of the binding of the mispairing-recognizing substrate is effected optically, by measuring the fluorescence of the fluorescence-labeled substrate, or by electrical readout, or by impedance measurement, or by surface plasmon resonance measurement, or by gravimetric measurement, or by cantilever or microcantilever or by acoustic methods.

30. The process as claimed in one of the preceding claims, wherein the successful hybridization of the nucleotide sequences being investigated is detected by a fluorescent dye, or by electronic detection, or by impedance measurement, or by surface plasmon resonance measurement, or gravimetrically, or using cantilever or microcantilever, or by means of acoustic methods.

31. The method as claimed in one of the preceding claims, wherein the sample nucleotide sequences and/or the reference nucleotide sequences and/or the mispairing-recognizing substrate are labeled differently.

32. The method as claimed in one of the preceding claims, wherein the fixing of the nucleotide sequences on the electronically addressable surface, the hybridization of the reference nucleotide sequences with the sample nucleotide sequences and the substrate binding are measured.

33. The method as claimed in one of claims 1 to 32, wherein the quantity of bound substrate is determined quantitatively.

34. The method for quantitatively detecting the expression of mRNA in different cells or tissues as claimed in claim 33, wherein
a) a known single-stranded nucleotide sequence is loaded onto a nucleotide chip,
b) labeled cDNA, which has been obtained from different cells or tissues, is likewise loaded onto the chip and a heteroduplex is produced by hybridization of the two sequences, and
c) the quantity of the mRNA is determined by quantitatively measuring the labeling.

35. The method as claimed in claim 33 or 34, wherein use is made of a dye-labeled cDNA and the color formed during the hybridization is measured optically quantitatively.

36. A method for preparing dye-labeled, mispairing-recognizing proteins, wherein the protein is incubated with a dye, which is present as an ester, in an aqueous solution and with the exclusion of light.

37. The method as claimed in claim 36, wherein the ester is employed at a concentration of between 1 μM and 100 μM.

38. The method as claimed in claim 36 or 37, wherein the ester employed is a dye-succinimidyl ester.

39. The method as claimed in one of claims 36 to 38, wherein use is made of a HEPES buffer consisting of 5 mM to 50 mM N-2-hydroxyethylpiperazine-N′-2-ethanesulfonic acid (HEPES), pH 7.5 to 8.5, 50 to 500 mM KCl, 1 to 15 mM MgCl₂, 5 to 15% glycerol in distilled water.

40. The method as claimed in one of claims 36 to 39, wherein a mispairing-binding protein as claimed in claim 18 is labeled.

41. The method as claimed in one of claims 36 to 40, wherein the mispairing-binding protein is labeled with a dye as claimed in claim 22.

42. A mispairing-recognizing protein, which is labeled by coupling to a detectable enzymic, antibody-binding, luminescent, radioactive, dye-carrying or fluorescent group.

43. A mispairing-recognizing protein as claimed in claim 42, which is a protein selected from the group consisting of mutS, mutY, MSH1 to MSH6, S1 nuclease, T4 endonuclease, thymine glycosylase and cleavase.

44. A mispairing-recognizing protein as claimed in claim 42 or 43, which is labeled with an enzymic group selected from the group consisting of chloramphenicol acetyltransferase, alkaline phosphatase, luciferase and peroxidase.

45. A mispairing-recognizing protein as claimed in claim 42 or 43, which is labeled with a fluorescent dye selected from the group consisting of Cy™3, Cy™5, Oregon Green 488, Alexa Fluor 488, Alexa Fluor 532, Alexa Fluor 546, Alexa Fluor 594, Alexa Fluor 647, Bodipy 558/568, Bodipy 650/665, Bodipy 564/570, S 0535, S 0536, Dy-630-NHS, Dy-635-NHS, EVOblue30-NHS, FAR-Blue, FAR-Fuchsia, Atto 650, FITC and Texas Red.

46. The use of mutS for a method for the site-resolved detection of mutations in nucleotide sequences on a support.

47. The use of mutS for a method for the detection of mutations in nucleotide sequences on an electronically addressable surface.

48. A kit comprising an electronically addressable chip, reference nucleotide sequences, a nuclease which degrades single-stranded nucleic acids, and at least one substrate which recognizes mispairings specifically.

49. A kit as claimed in claim 48, comprising an incubation buffer, a blocking buffer and a washing buffer.
